SEQUENCE LISTING (1 sequence)

SEQ ID NO: 1 - RNA, 15 nt, synthetic construct
  source 1..15: mol_type = other RNA
  modified_base 1..15: mod_base = OTHER; note = linked via phosphorothioate bonds
  modified_base 1: mod_base = gm
  modified_base 2: mod_base = cm
  modified_base 3: mod_base = OTHER; note = 2'-O-methyl-adenosine
  modified_base 4: mod_base = gm
  modified_base 5..6: mod_base = cm
  modified_base 7: mod_base = gm
  modified_base 8..10: mod_base = OTHER; note = 2'-O-methyl-adenosine
  modified_base 11..12: mod_base = cm
  modified_base 13: mod_base = gm
  modified_base 14: mod_base = OTHER; note = 2'-O-methyl-adenosine
  modified_base 15: mod_base = cm
gcagccgaaaccgac

(19) United States
(12) Patent Application Publication
(10) Pub. No.: US 2026/0041779 A1
(43) Pub. Date: Feb. 12, 2026

(54) NUCLEOTIDE-BASED ENHANCEMENT AGENT FOR RNA DELIVERY AND THERAPY

(71) Applicant: Sanegene Bio USA Inc., Woburn, MA (US)

(72) Inventors: Weimin WANG, Winchester, MA (US); Xiaochuan CAI, Bedford, MA (US); Shiyu WANG, Belmont, MA (US); Chunyang ZHANG, Burlington, MA (US)

(21) Appl. No.: 19/119,936

(22) PCT Filed: Nov. 2, 2023
(86) PCT No.: PCT/US2023/078442

§ 371 Date: Apr. 10, 2025

Related U.S. Application Data
(60) Provisional application No. 63/421,799, filed on Nov. 2, 2022.

(51) Int. Cl. A61K 47/64; A61K 47/54

(52) U.S. Cl. CPC A61K 47/64; A61K 47/549

(57) ABSTRACT

The present disclosure provides a Nucleotide-Based Enhancement Agent being a compound or a pharmaceutically acceptable salt thereof, comprising from 2 to 30 nucleotides, wherein each nucleotide independently is of Formula (I) or (II):

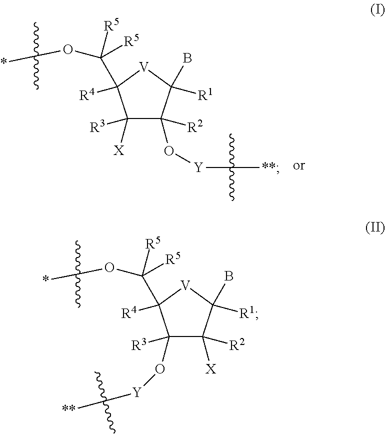

pharmaceutically acceptable salts thereof, and related conjugates. The present disclosure also relates to uses of the Nucleotide-Based Enhancement Agents and conjugates, e.g., in delivering nucleic acid and/or treating or preventing diseases.

### RELATED APPLICATION

This application claims priority to, and the benefit of, U.S. Application No. 63/421,799, filed on Nov. 2, 2022, the entire contents of which are incorporated herein by reference.

### INCORPORATION OF THE SEQUENCE LISTING

The contents of the electronic sequence listing (SANB_011_001WO_SeqList_ST26.xml; Size: 7,063 bytes; and Date of Creation: Oct. 31, 2023) are herein incorporated by reference in its entirety.

### BACKGROUND

Efficient delivery of genetic materials such as RNA to cells in vivo requires specific targeting and protection from the extracellular environment, particularly serum proteins. One method of achieving specific targeting is to conjugate a targeting moiety to the nucleic acid (e.g., oligonucleotide). The targeting moiety helps direct the nucleic acid to the site of interest. A targeting moiety can improve delivery by receptor-mediated endocytosis. This process is initiated via activation of a cell-surface or membrane receptor following binding of a specific ligand to the receptor. Many receptor-mediated endocytotic systems are known, including those that recognize sugars such as galactose, mannose, mannose-6-phosphate, peptides and proteins such as transferrin, asialoglycoprotein, vitamin B12, insulin, and epidermal growth factor (EGF). The asialoglycoprotein receptor (ASGP-R) is a high capacity receptor and is highly abundant on hepatocytes. The ASGP-R shows a high affinity for N-Acetyl-D-Galactosylamine (GalNAc) than D-Gal. Similarly, the glucagon-like peptide-1 receptor (GLP-1 receptor) is present in beta cells of the pancreas and can also be targeted with appropriate ligands to direct delivery. Recently, certain carbohydrate conjugates have been shown to be a valuable alternative to liposomes for nucleic acid delivery. Moreover, after successful delivery into cells, the stability of the nucleic acid in the cellular environment is important for achieving the desirable therapeutic effects.

Thus, there continues to be a need for novel modifications to enhance nucleic acid delivery. The present disclosure addresses this need.

### SUMMARY

In some aspects, the present disclosure provides a Nucleotide-Based Enhancement Agent being a compound or a pharmaceutically acceptable salt thereof, comprising from 2 to 30 nucleotides, wherein each nucleotide independently is of Formula (I) or (II):

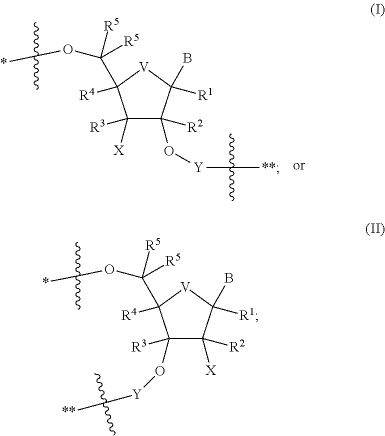

wherein:
each * independently denotes an attachment to the 2′- or 3′-position of another nucleotide of the Nucleotide-Based Enhancement Agent, or denotes H when the nucleotide is at the 5′-end of the Nucleotide-Based Enhancement Agent;
each ** independently denotes an attachment to the 5′-position of another nucleotide of the Nucleotide-Based Enhancement Agent, or denotes H when the nucleotide is at the 2′- or 3′-end of the Nucleotide-Based Enhancement Agent;
B is H, C₁-C₆ alkyl, or a nucleobase moiety;
V is —O—, —NR^{V}—, or —C(R^{V})₂—;
each R^{V} independently is H or C₁-C₆ alkyl;
X is H, halogen, or —OR^{X};
R^{X} is H, C₁-C₁₂ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Xa}; or R^{X} and R⁴ together form C₁-C₆ alkylene;
each R^{Xa} independently is halogen, C₁-C₆ alkyl, or —O—(C₁-C₆ alkyl), wherein the C₁-C₆ alkyl or —O—(C₁-C₆ alkyl) is optionally substituted with one or more halogen;
Y is —P(R^{Y})—, —P(OR^{Y})—, —P(N(R^{Y})₂)—, —P(═O)(OR^{Y})—, —P(═O)(R^{Y})—, —P(═S)(OR^{Y})—, —P(═S)(R^{Y})—, —P(═O)(SR^{Y})—, or —P(═S)(SR^{Y})—;
each R^{Y} independently is H or C₁-C₆ alkyl optionally substituted with one or more halogen or cyano;
R¹ is H, halogen, or C₁-C₆ alkyl optionally substituted with one or more halogen;
R² is H, halogen, or C₁-C₆ alkyl optionally substituted with one or more halogen;
R³ is H, halogen, or C₁-C₆ alkyl optionally substituted with one or more halogen;
R⁴ is H, halogen, or C₁-C₆ alkyl optionally substituted with one or more halogen; or R⁴ and R^{X} together form C₁-C₆ alkylene; and
each R⁵ independently is H, halogen, or C₁-C₆ alkyl optionally substituted with one or more halogen.

In some aspects, the present disclosure provides a conjugate or a pharmaceutically acceptable salt thereof, comprising:
(i) one or more Nucleic Acid Agent;
(ii) one or more Ligand; and
(iii) one or more Nucleotide-Based Enhancement Unit, wherein each Nucleotide-Based Enhancement Unit independently comprises from 2 to 30 nucleotides, wherein each nucleotide independently is of:

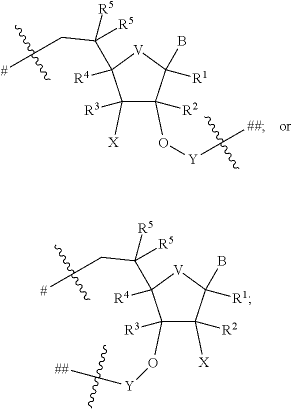

wherein:
variables B, V, X, Y, R¹, R², R³, R⁴, and R⁵ are described herein;
each # independently denotes an attachment to the 2′- or 3′-position of another nucleotide of the Nucleotide-Based Enhancement Unit, or denotes H or an attachment to the rest of the conjugate when the nucleotide is at the 5′-end of the Nucleotide-Based Enhancement Unit; and
each ## independently denotes an attachment to the 5′-position of another nucleotide of the Nucleotide-Based Enhancement Unit, or denotes H or an attachment to the rest of the conjugate when the nucleotide is at the 2′- or 3′-end of the Nucleotide-Based Enhancement Unit.

In some aspects, the present disclosure provides an isotopic derivative of a Nucleotide-Based Enhancement Agent described herein.

In some aspects, the present disclosure provides a pharmaceutical composition comprising a Nucleotide-Based Enhancement Agent or conjugate described herein.

In some aspects, the present disclosure provides a method of modulating the expression of a target gene in a subject, comprising administering to the subject a conjugate described herein.

In some aspects, the present disclosure provides a method of delivering a Nucleic Acid Agent to a subject, comprising administering to the subject a conjugate described herein.

In some aspects, the present disclosure provides a method of treating or preventing a disease in a subject in need thereof, comprising administering to the subject a therapeutically effective amount of a conjugate described herein.

In some aspects, the present disclosure provides a conjugate described herein for modulating the expression of a target gene in a subject.

In some aspects, the present disclosure provides a conjugate described herein for delivering a Nucleic Acid Agent to a subject.

In some aspects, the present disclosure provides a conjugate described herein for treating or preventing a disease in a subject in need thereof.

In some aspects, the present disclosure provides use of a conjugate described herein in the manufacture of a medicament for modulating the expression of a target gene in a subject.

In some aspects, the present disclosure provides use of a conjugate described herein in the manufacture of a medicament for delivering a Nucleic Acid Agent to a subject.

In some aspects, the present disclosure provides a use of a conjugate described herein in the manufacture of a medicament for treating or preventing a disease in a subject in need thereof.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1 is a graph showing various exemplary constructions of the conjugates described herein.

FIG. 2. is a graph showing the chemical structures of exemplary conjugates Duplex 1-5.

FIG. 3. is a graph showing the in vivo KD activity of Duplex 1-5 in a tissue of interest.

FIG. 4. are images of RNASCOPE-based mRNA silencing evaluation of Duplex 1-5 in a tissue of interest.

FIG. 5. are images of miRNASCOPE-based imaging of siRNA molecules accumulated in a tissue of interest.

### DETAILED DESCRIPTION

Efficient delivery of genetic materials such as RNA to cells in vivo requires specific targeting and protection from the extracellular environment, particularly serum proteins. One method of achieving specific targeting is to conjugate a targeting moiety to the nucleic acid (e.g., oligonucleotide). The targeting moiety may help direct the nucleic acid to the site of interest.

It is understood that oligonucleotides may need to circumvent several extracellular and intracellular barriers to achieve potent gene knockdown in specific tissues or cell types of disease. Chemical modifications and conjugation with targeting ligands could improve oligonucleotides' pharmacological properties and enhance tissue-specific delivery. However, previously, oligonucleotides conjugated with a single targeting ligand still failed to achieve clinically relevant knockdown activities and specificity, although a variety of conjugates with peptides, antibodies, small molecules, carbohydrates, and adapters have been tested. These suboptimal delivery approaches may result from the poor pharmacokinetic and ADME profiles of the conjugates, the lack of efficient tissue accumulation and cellular uptake, and the less efficient intracellular sorting and endosomal escape, among many other delivery obstacles.

The present disclosure provides Nucleotide-Based Enhancement Agents, which may improve the targeted delivery efficiency and enhance the nucleic acid's cellular activity, e.g., through the improved PK, stability, cellular uptake and trafficking, or other mechanisms. The present disclosure also provides conjugates comprising the Nucleotide-Based Enhancement Agent. The present disclosure also relates to uses of the Nucleotide-Based Enhancement Agents and conjugates, e.g., in delivering nucleic acid and/or treating or preventing diseases.

In some embodiments, the conjugate comprising the Nucleotide-Based Enhancement Agent has a higher targeted delivery efficiency as compared to a conjugate without the Nucleotide-Based Enhancement Agent.

In some aspects, the present disclosure provides a general delivery enhancement agent that could further improve the oligonucleotides' delivery efficiency, specificity, and knockdown activities, in the presence or absence of targeting ligands. The delivery enhancement agents could increase the conjugates' binding affinity with serum proteins, enhance their nucleases resistance, and improve pharmacokinetic properties. The delivery enhancement agents could also facilitate the oligonucleotides to traverse the capillary endothelium and to enter the tissue interstitium. The delivery enhancement agent could improve the oligonucleotide conjugates' binding with targeting receptors, thus improving the receptor-mediated endocytosis. The delivery enhancement agent could also facilitate the oligonucleotides' intracellular trafficking and endosomal escape, resulting improved intracellular activities. The delivery enhancement agent could improve the oligonucleotides' delivery and gene knockdown activities via other mechanisms or pathways.

### Nucleotide-Based Enhancement Agent

In some aspects, the present disclosure provides a Nucleotide-Based Enhancement Agent being a compound or a pharmaceutically acceptable salt thereof, comprising from 2 to 30 nucleotides, wherein each nucleotide independently is of Formula (I) or (II):

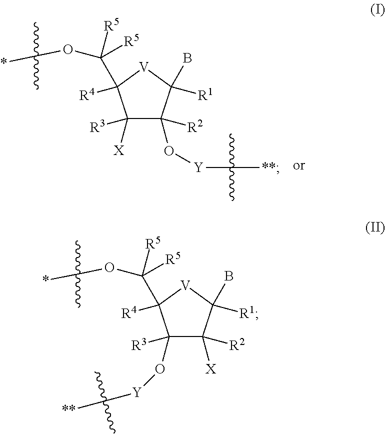

wherein:
each * independently denotes an attachment to the 2′- or 3′-position of another nucleotide of the Nucleotide-Based Enhancement Agent, or denotes H when the nucleotide is at the 5′-end of the Nucleotide-Based Enhancement Agent;
each ** independently denotes an attachment to the 5′-position of another nucleotide of the Nucleotide-Based Enhancement Agent, or denotes H when the nucleotide is at the 2′- or 3′-end of the Nucleotide-Based Enhancement Agent;
B is H, C₁-C₆ alkyl, or a nucleobase moiety;
V is —O—, —NR^{V}—, or —C(R^{V})—;
each R^{V} independently is H or C₁-C₆ alkyl;
X is H, halogen, or —OR^{X};
R^{X} is H, C₁-C₁₂ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Xa}; or R^{X} and R⁴ together form C₁-C₆ alkylene;
each R^{Xa} independently is halogen, C₁-C₆ alkyl, or —O—(C₁-C₆ alkyl), wherein the C₁-C₆ alkyl or —O—(C₁-C₆ alkyl) is optionally substituted with one or more halogen;
Y is —P(R^{Y})—, —P(OR^{Y})—, —P(N(R^{Y})₂)—, —P(═O)(OR^{Y})—, —P(═O)(R^{Y})—, —P(═S)(OR^{Y})—, —P(═S)(R^{Y})—, —P(═O)(SR^{Y})—, or —P(═S)(SR^{Y})—;
each R^{Y} independently is H or C₁-C₆ alkyl optionally substituted with one or more halogen or cyano;
R¹ is H, halogen, or C₁-C₆ alkyl optionally substituted with one or more halogen;
R² is H, halogen, or C₁-C₆ alkyl optionally substituted with one or more halogen;
R³ is H, halogen, or C₁-C₆ alkyl optionally substituted with one or more halogen;
R⁴ is H, halogen, or C₁-C₆ alkyl optionally substituted with one or more halogen; or R⁴ and R^{X} together form C₁-C₆ alkylene; and
each R⁵ independently is H, halogen, or C₁-C₆ alkyl optionally substituted with one or more halogen.

In some embodiments, the Nucleotide-Based Enhancement Agent comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides.

Tn some embodiments, the Nucleotide-Based Enhancement Agent comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides.

In some embodiments, the Nucleotide-Based Enhancement Agent comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides.

In some embodiments, the Nucleotide-Based Enhancement Agent comprises 5 nucleotides. In some embodiments, the Nucleotide-Based Enhancement Agent comprises 6 nucleotides. In some embodiments, the Nucleotide-Based Enhancement Agent comprises 7 nucleotides. In some embodiments, the Nucleotide-Based Enhancement Agent comprises 8 nucleotides. In some embodiments, the Nucleotide-Based Enhancement Agent comprises 9 nucleotides. In some embodiments, the Nucleotide-Based Enhancement Agent comprises 10 nucleotides. In some embodiments, the Nucleotide-Based Enhancement Agent comprises 11 nucleotides. In some embodiments, the Nucleotide-Based Enhancement Agent comprises 12 nucleotides. In some embodiments, the Nucleotide-Based Enhancement Agent comprises 13 nucleotides.

In some embodiments, the Nucleotide-Based Enhancement Agent comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides.

In some embodiments, the Nucleotide-Based Enhancement Agent comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Agent comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides.

In some embodiments, the Nucleotide-Based Enhancement Agent comprises one or more phosphodiester linkers and/or one or more phosphorothioate linkers. In some embodiments, at least two nucleotides of the Nucleotide-Based Enhancement Agent are connected via a phosphodiester linker or a phosphorothioate linker. In some embodiments, at least two nucleotides of the Nucleotide-Based Enhancement Agent are connected via a phosphorothioate linker. In some embodiments, each two adjacent nucleotides of the Nucleotide-Based Enhancement Agent are connected via a phosphodiester linker or a phosphorothioate linker.

In some embodiments, the Nucleotide-Based Enhancement Agent comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides, wherein the Nucleotide-Based Enhancement Agent further comprises one or more phosphodiester linkers and/or one or more phosphorothioate linkers.

In some embodiments, the Nucleotide-Based Enhancement Agent comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides, wherein the Nucleotide-Based Enhancement Agent further comprises one or more phosphodiester linkers and/or one or more phosphorothioate linkers.

In some embodiments, the Nucleotide-Based Enhancement Agent comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides, wherein the Nucleotide-Based Enhancement Agent further comprises one or more phosphodiester linkers and/or one or more phosphorothioate linkers.

In some embodiments, the Nucleotide-Based Enhancement Agent comprises 5 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 6 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 7 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 8 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 9 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 10 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 11 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 12 nucleotides, wherein the Nucleotide-Based Enhancement Agent further comprises one or more phosphodiester linkers and/or one or more phosphorothioate linkers. In some embodiments, the Nucleotide-Based Enhancement Agent comprises 13 nucleotides, wherein the Nucleotide-Based Enhancement Agent further comprises one or more phosphodiester linkers and/or one or more phosphorothioate linkers.

In some embodiments, the Nucleotide-Based Enhancement Agent comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides, wherein the Nucleotide-Based Enhancement Agent further comprises one or more phosphodiester linkers and/or one or more phosphorothioate linkers.

In some embodiments, the Nucleotide-Based Enhancement Agent comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Agent comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides, wherein the Nucleotide-Based Enhancement Agent further comprises one or more phosphodiester linkers and/or one or more phosphorothioate linkers.

In some embodiments, the Nucleotide-Based Enhancement Agent comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides, wherein the Nucleotide-Based Enhancement Agent further comprises one or more phosphodiester linkers.

In some embodiments, the Nucleotide-Based Enhancement Agent comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides, wherein the Nucleotide-Based Enhancement Agent further comprises one or more phosphodiester linkers.

In some embodiments, the Nucleotide-Based Enhancement Agent comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides, wherein the Nucleotide-Based Enhancement Agent further comprises one or more phosphodiester linkers.

In some embodiments, the Nucleotide-Based Enhancement Agent comprises 5 nucleotides, wherein the Nucleotide-Based Enhancement Agent further comprises one or more phosphodiester linkers. In some embodiments, the Nucleotide-Based Enhancement Agent comprises 6 nucleotides, wherein the Nucleotide-Based Enhancement Agent further comprises one or more phosphodiester linkers. In some embodiments, the Nucleotide-Based Enhancement Agent comprises 7 nucleotides, wherein the Nucleotide-Based Enhancement Agent further comprises one or more phosphodiester linkers. In some embodiments, the Nucleotide-Based Enhancement Agent comprises 8 nucleotides, wherein the Nucleotide-Based Enhancement Agent further comprises one or more phosphodiester linkers. In some embodiments, the Nucleotide-Based Enhancement Agent comprises 9 nucleotides, wherein the Nucleotide-Based Enhancement Agent further comprises one or more phosphodiester linkers. In some embodiments, the Nucleotide-Based Enhancement Agent comprises 10 nucleotides, wherein the Nucleotide-Based Enhancement Agent further comprises one or more phosphodiester linkers. In some embodiments, the Nucleotide-Based Enhancement Agent comprises 11 nucleotides, wherein the Nucleotide-Based Enhancement Agent further comprises one or more phosphodiester linkers. In some embodiments, the Nucleotide-Based Enhancement Agent comprises 12 nucleotides, wherein the Nucleotide-Based Enhancement Agent further comprises one or more phosphodiester linkers. In some embodiments, the Nucleotide-Based Enhancement Agent comprises 13 nucleotides, wherein the Nucleotide-Based Enhancement Agent further comprises one or more phosphodiester linkers.

In some embodiments, the Nucleotide-Based Enhancement Agent comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides, wherein the Nucleotide-Based Enhancement Agent further comprises one or more phosphodiester linkers.

In some embodiments, the Nucleotide-Based Enhancement Agent comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Agent comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides, wherein the Nucleotide-Based Enhancement Agent further comprises one or more phosphodiester linkers.

In some embodiments, the Nucleotide-Based Enhancement Agent comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides, wherein the Nucleotide-Based Enhancement Agent further comprises one or more phosphorothioate linkers.

In some embodiments, the Nucleotide-Based Enhancement Agent comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides, wherein the Nucleotide-Based Enhancement Agent further comprises one or more phosphorothioate linkers.

In some embodiments, the Nucleotide-Based Enhancement Agent comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides, wherein the Nucleotide-Based Enhancement Agent further comprises one or more phosphorothioate linkers.

In some embodiments, the Nucleotide-Based Enhancement Agent comprises 5 nucleotides, wherein the Nucleotide-Based Enhancement Agent further comprises one or more phosphorothioate linkers. In some embodiments, the Nucleotide-Based Enhancement Agent comprises 6 nucleotides, wherein the Nucleotide-Based Enhancement Agent further comprises one or more phosphorothioate linkers. In some embodiments, the Nucleotide-Based Enhancement Agent comprises 7 nucleotides, wherein the Nucleotide-Based Enhancement Agent further comprises one or more phosphorothioate linkers. In some embodiments, the Nucleotide-Based Enhancement Agent comprises 8 nucleotides, wherein the Nucleotide-Based Enhancement Agent further comprises one or more phosphorothioate linkers. In some embodiments, the Nucleotide-Based Enhancement Agent comprises 9 nucleotides, wherein the Nucleotide-Based Enhancement Agent further comprises one or more phosphorothioate linkers. In some embodiments, the Nucleotide-Based Enhancement Agent comprises 10 nucleotides, wherein the Nucleotide-Based Enhancement Agent further comprises one or more phosphorothioate linkers. In some embodiments, the Nucleotide-Based Enhancement Agent comprises 11 nucleotides, wherein the Nucleotide-Based Enhancement Agent further comprises one or more phosphorothioate linkers. In some embodiments, the Nucleotide-Based Enhancement Agent comprises 12 nucleotides, wherein the Nucleotide-Based Enhancement Agent further comprises one or more phosphorothioate linkers. In some embodiments, the Nucleotide-Based Enhancement Agent comprises 13 nucleotides, wherein the Nucleotide-Based Enhancement Agent further comprises one or more phosphorothioate linkers.

In some embodiments, the Nucleotide-Based Enhancement Agent comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides, wherein the Nucleotide-Based Enhancement Agent further comprises one or more phosphorothioate linkers.

In some embodiments, the Nucleotide-Based Enhancement Agent comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Agent comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides, wherein the Nucleotide-Based Enhancement Agent further comprises one or more phosphorothioate linkers.

It is understood that, for a compound (e.g., a Nucleotide-Based Enhancement Agent or conjugate) of the present disclosure, variables B, V, R^{V}, X, R^{X}, R^{Xa}, Y, R^{Y}, R¹, R², R³, R⁴, and R⁵ can each be, where applicable, selected from the groups described herein, and any group described herein for any of variables B, V, R^{V}, X, R^{X}, R^{Xa}, Y, R^{Y}, R¹, R², R³, R⁴, and R⁵ can be combined, where applicable, with any group described herein for one or more of the remainder of variables B, V, R^{V}, X, R^{X}, R^{xa}, Y, R, R¹, R², R³, R⁴, and R⁵.

### Variable B

In some embodiments, B is H.

Tn some embodiments, B is C₁-C₆ alkyl (e.g., methyl, ethyl, n-propyl, i-propyl, n-butyl, i-butyl, s-butyl, t-butyl, pentyl, or hexyl).

Tn some embodiments, B is methyl, ethyl, or propyl.

In some embodiments, B is a nucleobase moiety.

The term, “nucleobase moiety”, as used herein, refers to a nucleobase that is attached to the rest of the compound, e.g., via an atom of the nucleobase or a functional group thereof.

In some embodiments, the nucleobase moiety is adenine (A), cytosine (C), guanine (G), thymine (T), or uracil (U).

In some embodiments, the nucleobase moiety is

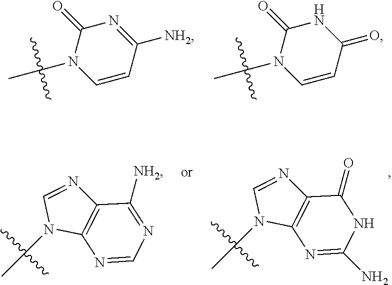 [SMILES: *n1ccc(N)nc1=O ; *n1ccc(=O)[nH]c1=O ; *n1cnc2c(N)ncnc21 ; *n1cnc2c(=O)[nH]c(N)nc21]

wherein

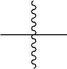

indicates the point of attachment to the rest of the Nucleotide-Based Enhancement Agent.

In some embodiments, the nucleobase moiety is a modified nucleobase.

In some embodiments, the modified nucleobase is 5-methylcytosine.

In some embodiments, the modified nucleobase is hypoxanthine, xanthine, or 7-methylguanine.

In some embodiments, the modified nucleobase is 5,6-dihydrouracil, 5-methylcytosine, or 5-hydroxymethylcytosine.

In some embodiments, the nucleobase moiety is an artificial nucleobase.

In some embodiments, the artificial nucleobase is isoguanine, isocytosine, 2-amino-6-(2-thienyl)purine, or pyrrole-2-carbaldehyde.

### Variable V and R^{1′}

In some embodiments, V is —O—.

In some embodiments, V is —NR^{V}—.

In some embodiments, V is —NH—.

In some embodiments, V is —C(R^{V})₂—.

In some embodiments, V is —CH₂—.

In some embodiments, at least one R^{V} is H.

Tn some embodiments, each R^{V} is H.

In some embodiments, at least one R^{V} is C₁-C₆ alkyl (e.g., methyl, ethyl, n-propyl, i-propyl, n-butyl, i-butyl, s-butyl, t-butyl, pentyl, or hexyl).

In some embodiments, each R^{V} is C₁-C₆ alkyl (e.g., methyl, ethyl, n-propyl, i-propyl, n-butyl, i-butyl, s-butyl, t-butyl, pentyl, or hexyl).

Variable X, R^{X}, R^{Xa}, Y, and R^{Y}

In some embodiments, X is H.

In some embodiments, X is halogen (e.g., F, Cl, Br, or I).

In some embodiments, X is F or Cl.

In some embodiments, X is F.

In some embodiments, X is —OR^{X}.

In some embodiments, X is —OH.

In some embodiments, X is —O—(C₁-C₁₂ alkyl).

In some embodiments, X is —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl).

In some embodiments, X is —OCH₃.

In some embodiments, X is —O—(C₁-C₆ alkyl)-O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl).

In some embodiments, X is —OCH₂CH₂OCH₃.

In some embodiments, X is —O—(C₁-C₆ alkyl)-(C₆-C₁₀ aryl) optionally substituted with one or more R^{X}.

In some embodiments, X is —O—(C₁-C₆ alkyl)-(C₆-C₁₀ aryl).

In some embodiments, X is

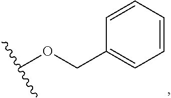 [SMILES: *OCc1ccccc1]

wherein

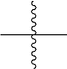

indicates the point of attachment to the rest of the Nucleotide-Based Enhancement Agent.

In some embodiments, X is

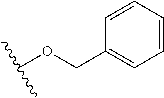 [SMILES: *OCc1ccccc1]

optionally substituted with one or more R^{Xa}, wherein

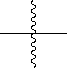

indicates the point of attachment to the rest of the Nucleotide-Based Enhancement Agent.

In some embodiments, X is

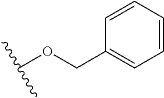 [SMILES: *OCc1ccccc1]

is optionally substituted with one or more halogen, wherein

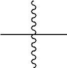

indicates the point of attachment to the rest of the Nucleotide-Based Enhancement Agent.

In some embodiments, X is

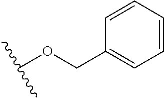 [SMILES: *OCc1ccccc1]

optionally substituted with one or more C₁-C₆ alkyl or —O—(C₁-C₆ alkyl), wherein the C₁-C₆ alkyl or —O—(C₁-C₆ alkyl) is optionally substituted with one or more halogen, and

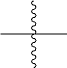

indicates the point of attachment to the rest of the Nucleotide-Based Enhancement Agent.

In some embodiments, R^{X} is H.

In some embodiments, R^{X} is C₁-C₁₂ alkyl optionally substituted with one or more R^{Xa}.

In some embodiments, R^{X} is C₁-C₁₂ alkyl.

In some embodiments, R^{X} is C₁-C₆ alkyl (e.g., methyl, ethyl, n-propyl, i-propyl, n-butyl, i-butyl, s-butyl, t-butyl, pentyl, or hexyl) optionally substituted with one or more R^{Xa}.

In some embodiments, R^{X} is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I) or —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl) optionally substituted with one or more halogen.

In some embodiments, R^{X} is C₁-C₆ alkyl (e.g., methyl, ethyl, n-propyl, i-propyl, n-butyl, i-butyl, s-butyl, t-butyl, pentyl, or hexyl).

In some embodiments, R^{X} is methyl, ethyl, or propyl.

In some embodiments, R^{X} is methyl.

In some embodiments, R^{X} is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, R^{X} is C₁-C₆ alkyl (e.g., methyl, ethyl, n-propyl, i-propyl, n-butyl, i-butyl, s-butyl, t-butyl, pentyl, or hexyl) substituted with one or more —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl), wherein the —O—(C₁-C₆ alkyl) is optionally substituted with one or more halogen.

In some embodiments, R^{X} is —(C₁-C₆ alkyl)-(C₆-C₁₀ aryl) optionally substituted with one or more R^{Xa}.

In some embodiments, R^{X} is —(C₁-C₆ alkyl)-(C₆-C₁₀ aryl) optionally substituted with one or more halogen (e.g., F, Cl, Br, or 1), C₁-C₆ alkyl (e.g., methyl, ethyl, n-propyl, i-propyl, n-butyl, i-butyl, s-butyl, t-butyl, pentyl, or hexyl), or —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl), wherein the C₁-C₆ alkyl or —O—(C₁-C₆ alkyl) is optionally substituted with one or more halogen.

In some embodiments, R^{X} is —(C₁-C₆ alkyl)-(C₆-C₁₀ aryl).

In some embodiments, R^{X} and R⁴ together form C₁-C₆ alkylene (e.g., methylene, ethylene, propylene, butylene, pentylene, or hexylene).

In some embodiments, R^{X} and R⁴ together form methylene, ethylene, or propylene.

In some embodiments, R^{X} and R⁴ together form methylene.

In some embodiments, R^{X} and R⁴ together form ethylene.

In some embodiments, R^{X} and R⁴ together form propylene.

In some embodiments, Y is —P(R^{Y})—.

In some embodiments, Y is —PH—.

In some embodiments, Y is —P(OR^{Y})—.

In some embodiments, Y is —P(OH)—.

In some embodiments, Y is —P(N(R^{Y})₂)—.

In some embodiments, Y is —P(NH₂)—.

In some embodiments, Y is —P(═O)(OR^{Y})—.

In some embodiments, Y is —P(═O)(OH)—.

In some embodiments, Y is —P(═O)(R^{Y})—.

In some embodiments, Y is —P(═O)H—.

In some embodiments, Y is —P(═S)(OR^{Y})—.

In some embodiments, Y is —P(═S)(OH)—.

In some embodiments, Y is —P(═S)(R^{Y})—.

In some embodiments, Y is —P(═S)H—.

In some embodiments, Y is —P(═O)(SR^{Y})—.

In some embodiments, Y is —P(═O)(SH)—.

In some embodiments, Y is —P(═S)(SR^{Y})—.

In some embodiments, Y is —P(═S)(SH)—.

In some embodiments, at least one R^{Y} is H.

In some embodiments, each R^{Y} is H.

In some embodiments, at least one R^{Y} is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I) or cyano.

In some embodiments, each R^{Y} is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I) or cyano.

In some embodiments, at least one R^{Y} is H, and at least one R^{Y} is C₁-C₆ alkyl (e.g., methyl, ethyl, n-propyl, i-propyl, n-butyl, i-butyl, s-butyl, t-butyl, pentyl, or hexyl) optionally substituted with one or more halogen or cyano.

Variables R¹, R², R³, R⁴, and R⁵

In some embodiments, R¹ is H.

In some embodiments, R¹ is halogen (e.g., F, Cl, Br, or I).

In some embodiments, R¹ is F or Cl.

In some embodiments, R¹ is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I).

In some embodiments, R¹ is C₁-C₆ alkyl (e.g., methyl, ethyl, n-propyl, i-propyl, n-butyl, i-butyl, s-butyl, t-butyl, pentyl, or hexyl).

In some embodiments, R¹ is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, R² is H.

In some embodiments, R² is halogen (e.g., F, Cl, Br, or I).

In some embodiments, R² is F or Cl.

In some embodiments, R² is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I).

In some embodiments, R² is C₁-C₆ alkyl (e.g., methyl, ethyl, n-propyl, i-propyl, n-butyl, i-butyl, s-butyl, t-butyl, pentyl, or hexyl).

In some embodiments, R² is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, R³ is H.

In some embodiments, R³ is halogen (e.g., F, Cl, Br, or I).

In some embodiments, R³ is F or Cl.

In some embodiments, R³ is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or 1).

In some embodiments, R³ is C₁-C₆ alkyl (e.g., methyl, ethyl, n-propyl, i-propyl, n-butyl, i-butyl, s-butyl, t-butyl, pentyl, or hexyl).

In some embodiments, R³ is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or 1).

In some embodiments, R⁴ is H.

In some embodiments, R⁴ is halogen (e.g., F, Cl, Br, or I).

In some embodiments, R⁴ is F or Cl.

In some embodiments, R⁴ is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I).

In some embodiments, R⁴ is C₁-C₆ alkyl (e.g., methyl, ethyl, n-propyl, i-propyl, n-butyl, i-butyl, s-butyl, t-butyl, pentyl, or hexyl).

In some embodiments, R⁴ is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, R⁴ and R^{X} together form C₁-C₆ alkylene (e.g., methylene, ethylene, propylene, butylene, pentylene, or hexylene).

In some embodiments, R⁴ and R^{X} together form methylene, ethylene, or propylene.

In some embodiments, R⁴ and R^{X} together form methylene.

In some embodiments, R⁴ and R^{X} together form ethylene.

In some embodiments, R⁴ and R^{X} together form propylene.

In some embodiments, each R⁵ is H.

In some embodiments, at least one R⁵ is halogen (e.g., F, Cl, Br, or I) or C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I).

In some embodiments, at least one R⁵ is halogen (e.g., F, Cl, Br, or I).

In some embodiments, at least one R⁵ is F or Cl.

In some embodiments, at least one R⁵ is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I).

In some embodiments, at least one R⁵ is C₁-C₆ alkyl (e.g., methyl, ethyl, n-propyl, i-propyl, n-butyl, i-butyl, s-butyl, t-butyl, pentyl, or hexyl).

In some embodiments, at least one R⁵ is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, each of R¹, R², R³, R⁴, and R⁵ is H.

### Exemplary Embodiments of the Nucleotides of the Nucleotide-Based Enhancement Agents

In some embodiments, at least one nucleotide of the Nucleotide-Based Enhancement Agent is of Formula (I′) or (II′):

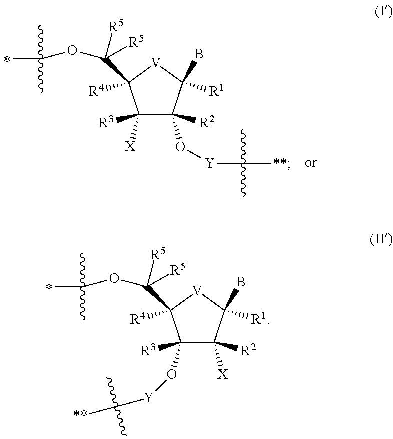

In some embodiments, at least one nucleotide of the Nucleotide-Based Enhancement Agent is of Formula (I-A), (II-A), (III-A), or (IV-A):

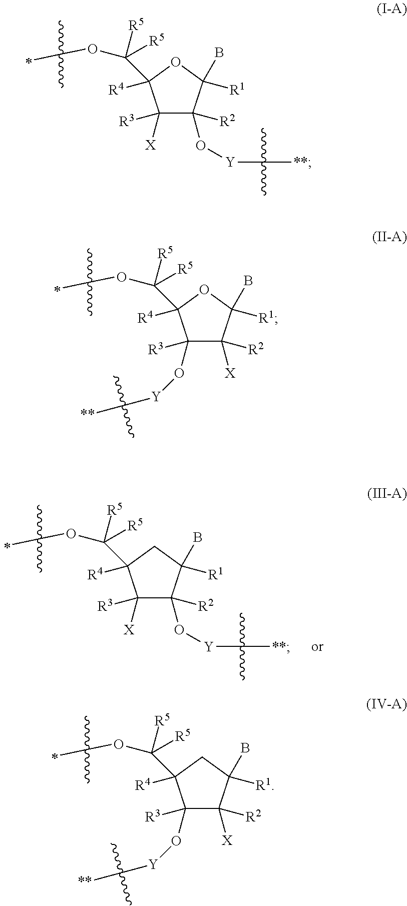

In some embodiments, at least one nucleotide of the Nucleotide-Based Enhancement Agent is of Formula (I′-A), (II′-A), (III′-A), or (IV′-A):

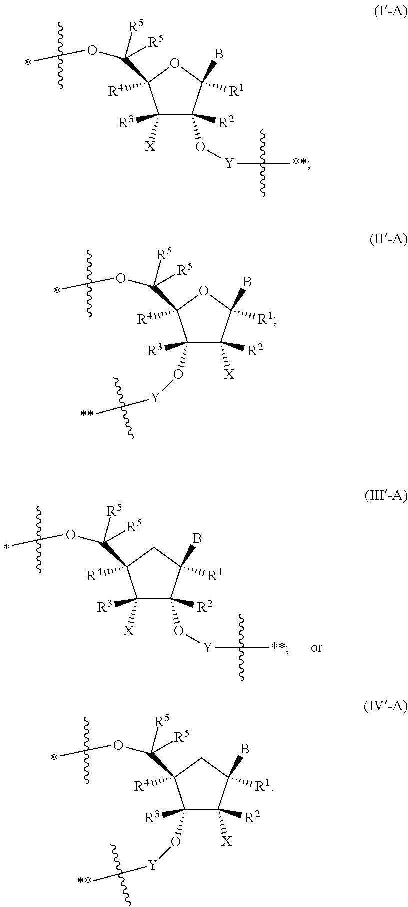

In some embodiments, at least one nucleotide of the Nucleotide-Based Enhancement Agent is of Formula (I-B) or (II-B):

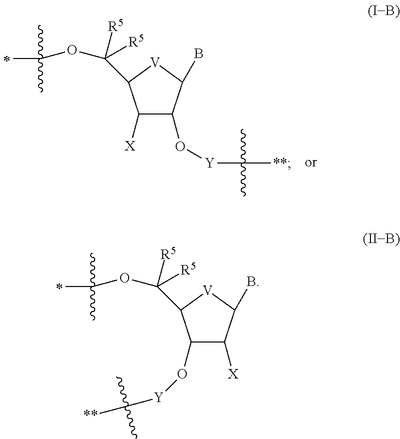

In some embodiments, at least one nucleotide of the Nucleotide-Based Enhancement Agent is of Formula (I′-B) or (II′-B):

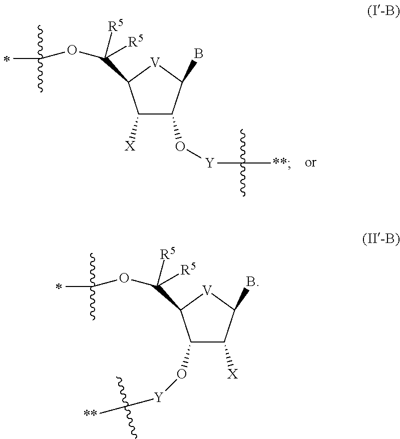

In some embodiments, at least one nucleotide of the Nucleotide-Based Enhancement Agent is of Formula (I-C), (II-C), (III-C), or (IV-C):

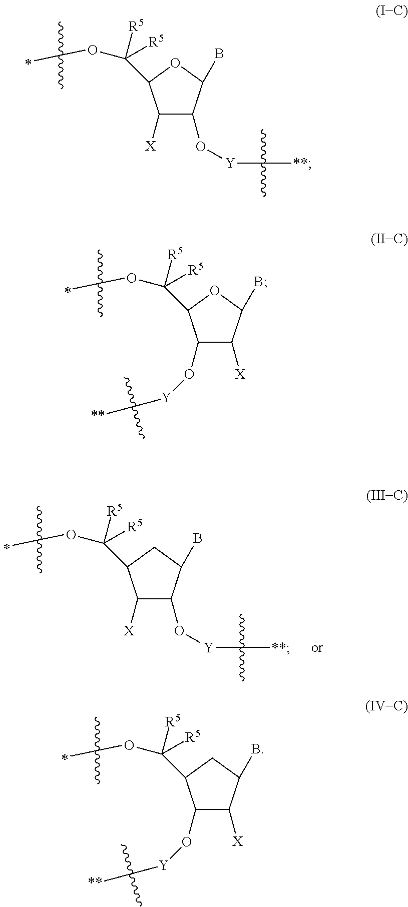

In some embodiments, at least one nucleotide of the Nucleotide-Based Enhancement Agent is of Formula (I′-C), (II′-C), (III′-C), or (IV′-C):

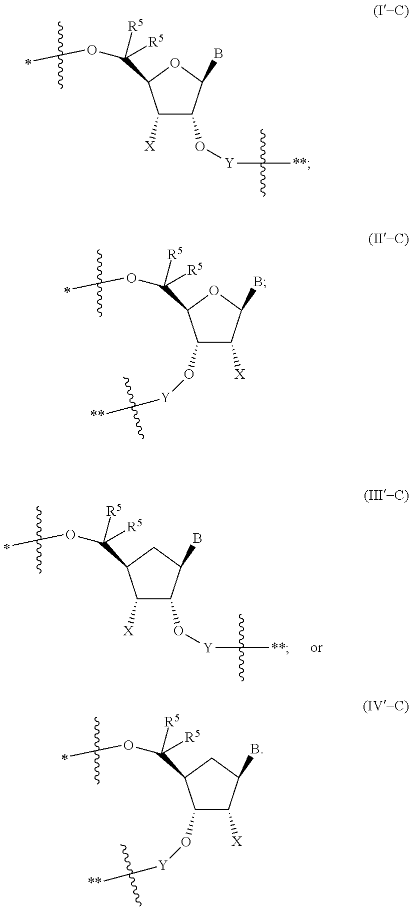

In some embodiments, at least one nucleotide of the Nucleotide-Based Enhancement Agent is selected from the nucleotides described in Table I.

TABLE L

| | | Se- |
|---|---|---|
| | | quence |
| No. | Structure | Code |
| L-1 | 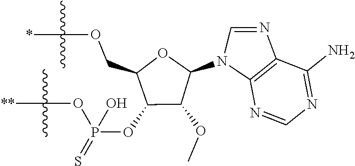 | [mAs] |
| L-2 | 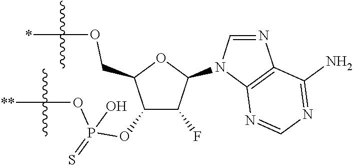 | [fAs] |
| L-3 | 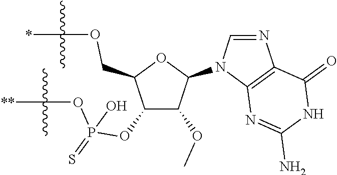 | [mGs] |
| L-4 | 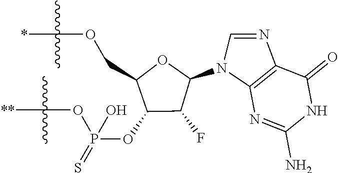 | [fGs] |
| L-5 | 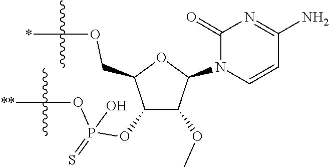 | [mCs] |
| L-6 | 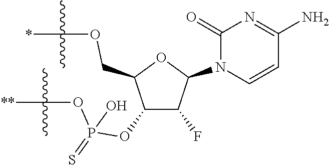 | [fCs] |
| L-7 | 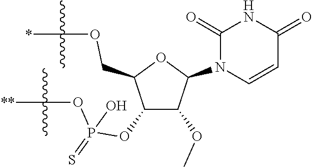 | [mUs] |
| L-8 | 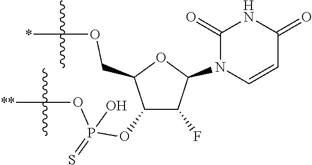 | [fUs] |
| L-9 | 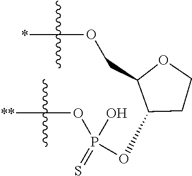 | [Abs] |
| L-10 | 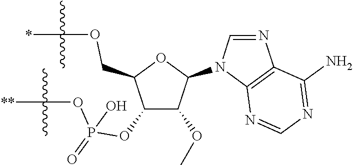 | [mA] |
| L-11 | 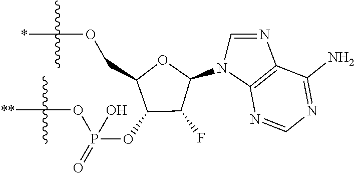 | [fA] |
| L-12 | 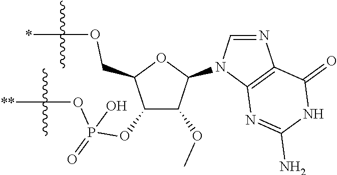 | [mG] |
| L-13 | 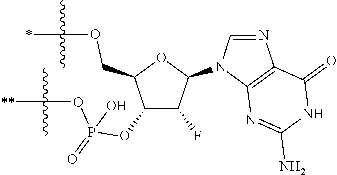 | [fG] |
| L-14 | 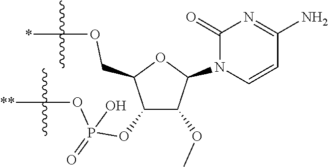 | [mC] |
| L-15 | 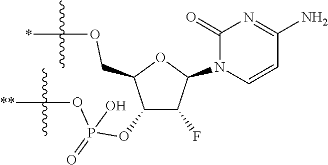 | [fC] |
| L-16 | 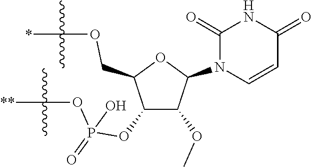 | [mU] |
| L-17 | 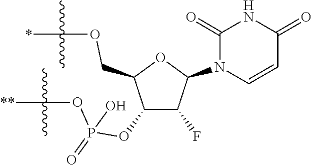 | [fU] |
| L-18 | 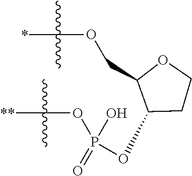 | [Ab] |
| L-19 | 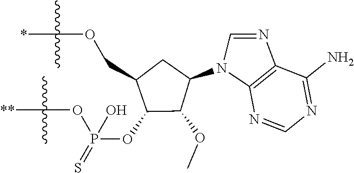 | |
| L-20 | 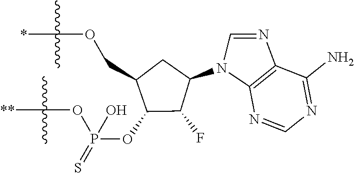 | |
| L-21 | 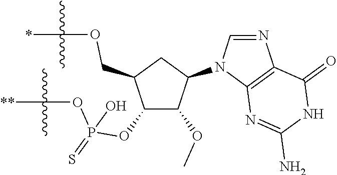 | |
| L-22 | 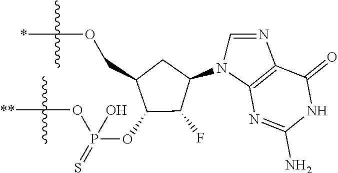 | |
| L-23 | 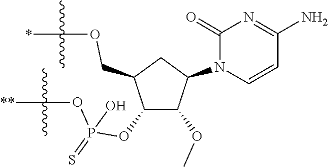 | |
| L-24 | 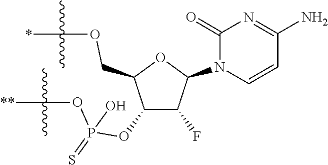 | |
| L-25 | 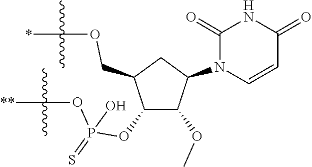 | |
| L-26 | 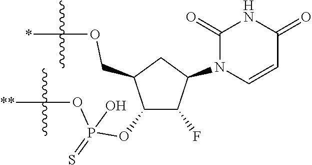 | |
| L-27 | 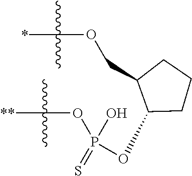 | |
| L-28 | 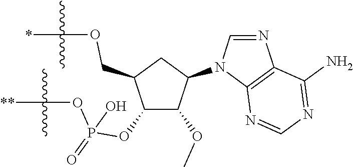 | |
| L-29 | 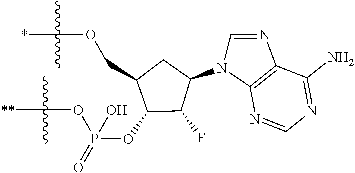 | |
| L-30 | 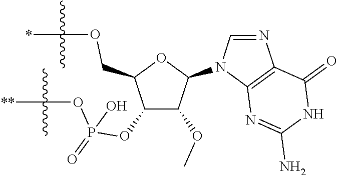 | |
| L-31 | 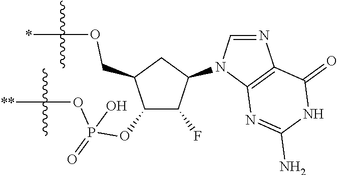 | |
| L-32 | 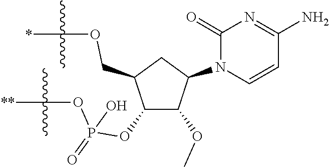 | |
| L-33 | 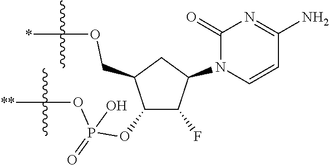 | |
| L-34 | 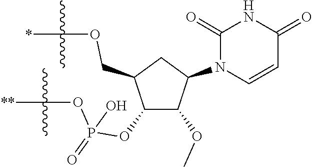 | |
| L-35 | 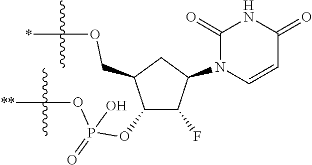 | |
| L-36 | 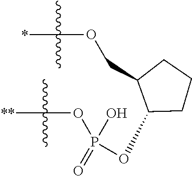 | |
| L-37 | 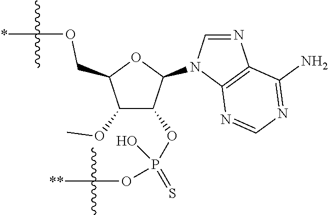 | |
| L-38 | 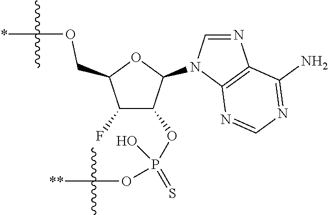 | |
| L-39 | 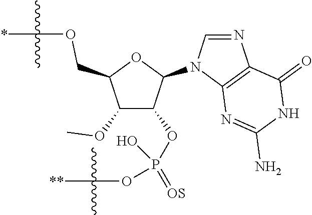 | |
| L-40 | 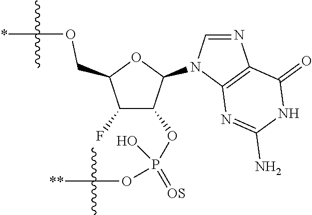 | |
| L-41 | 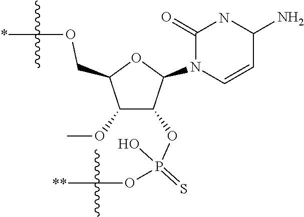 | |
| L-42 | 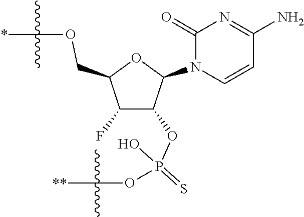 | |
| L-43 | 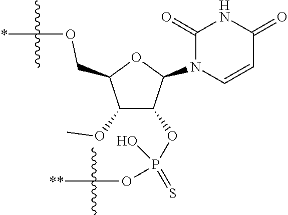 | |
| L-44 | 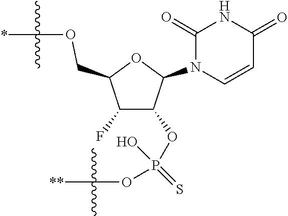 | |
| L-45 | 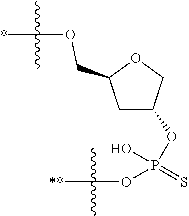 | |
| L-46 | 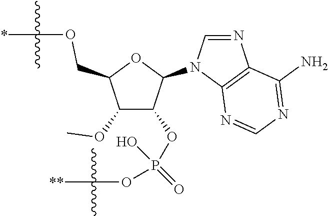 | |
| L-47 | 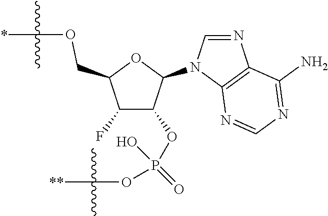 | |
| L-48 | 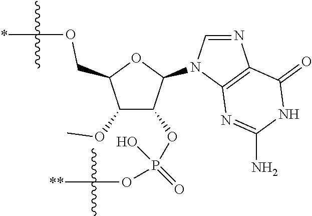 | |
| L-49 | 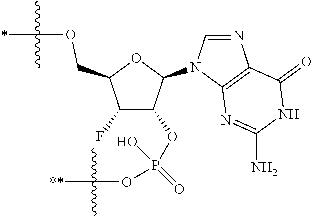 | |
| L-50 | 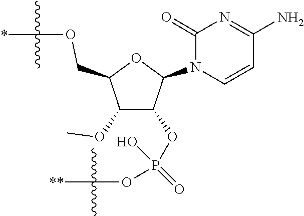 | |
| L-51 | 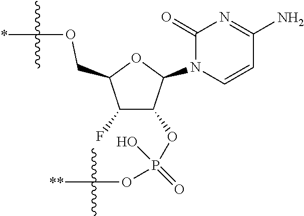 | |
| L-52 | 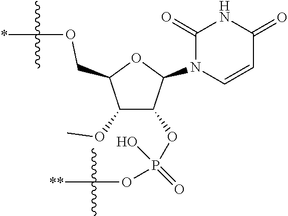 | |
| L-53 | 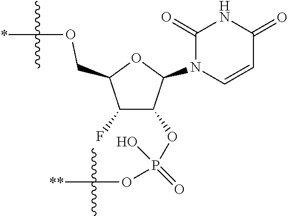 | |
| L-54 | 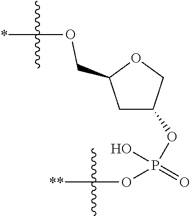 | |
| L-55 | 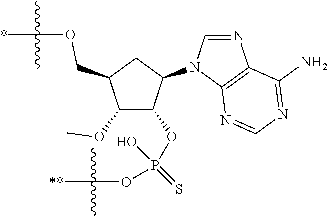 | |
| L-56 | 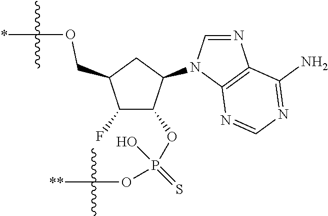 | |
| L-57 | 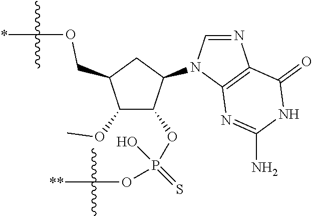 | |
| L-58 | 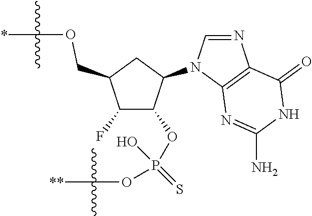 | |
| L-59 | 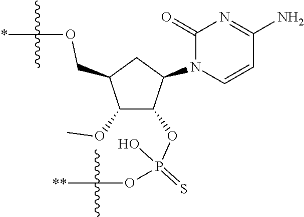 | |
| L-60 | 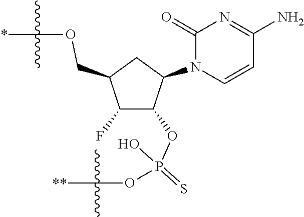 | |
| L-61 | 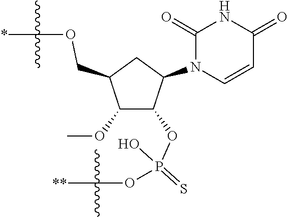 | |
| L-62 | 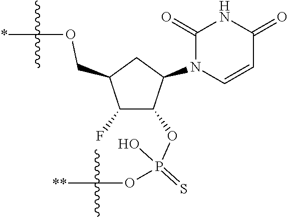 | |
| L-63 | 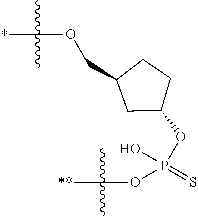 | |
| L-64 | 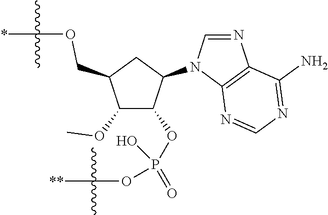 | |
| L-65 | 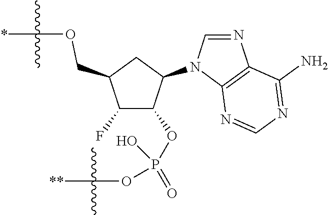 | |
| L-66 | 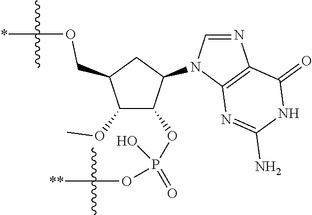 | |
| L-67 | 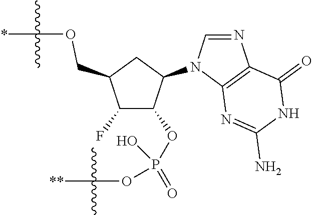 | |
| L-68 | 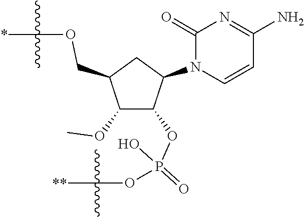 | |
| L-69 | 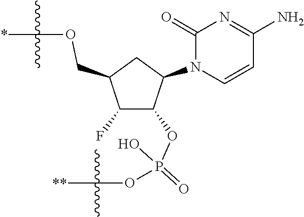 | |
| L-70 | 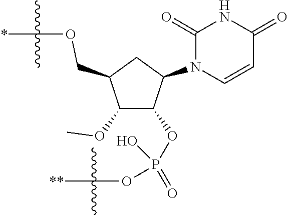 | |
| L-71 | 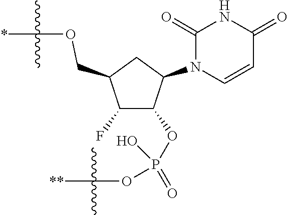 | |
| L-72 | 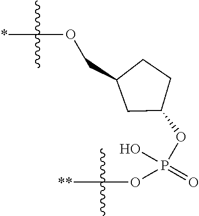 | |
| L-73 | 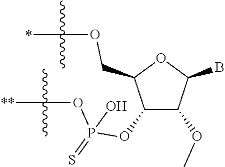 | |
| L-74 | 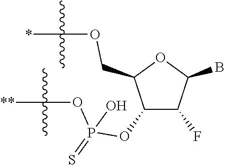 | |
| L-75 | 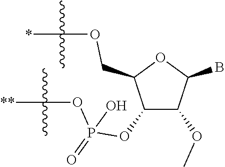 | |
| L-76 | 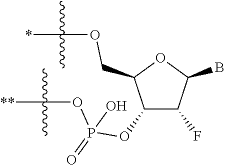 | |
| L-77 | 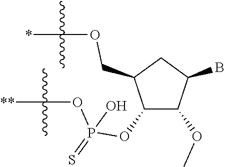 | |
| L-78 | 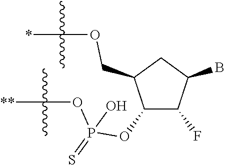 | |
| L-79 | 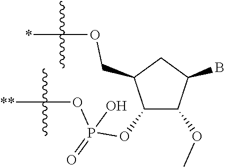 | |
| L-80 | 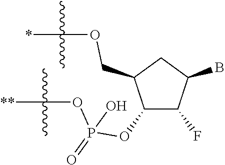 | |
| L-81 | 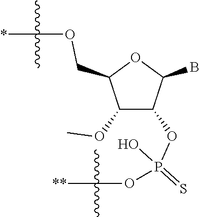 | |
| L-82 | 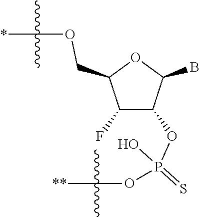 | |
| L-83 | 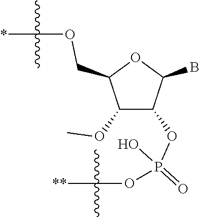 | |
| L-84 | 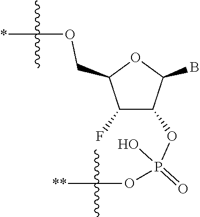 | |
| L-85 | 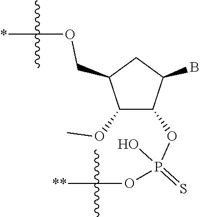 | |
| L-86 | 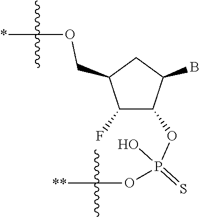 | |
| L-87 | 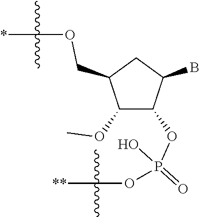 | |
| L-88 | 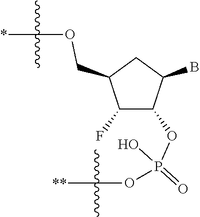 | |

In some aspects, the present disclosure provides an isotopic derivative (e.g., isotopically labeled compound) of a Nucleotide-Based Enhancement Agent disclosed herein.

It is understood that the isotopic derivative can be prepared using any of a variety of art-recognized techniques. For example, the isotopic derivative can generally be prepared by carrying out the procedures disclosed in the Schemes and/or in the Examples herein, by substituting an isotopically labeled reagent for a non-isotopically labeled reagent.

In some embodiments, the isotopic derivative is a deuterium labeled derivative.

The term “isotopic derivative”, as used herein, refers to a derivative of a chemical structure in which one or more atoms are isotopically enriched or labelled. For example, an isotopic derivative of an agent is isotopically enriched with regard to, or labelled with, one or more isotopes as compared to the corresponding agent. In some embodiments, the isotopic derivative is enriched with regard to, or labelled with, one or more atoms selected from ²H, ¹³C, ¹⁴C, ¹⁵N, ¹⁸O, ²⁹Si, ³²P, and ³⁴S. In some embodiments, the isotopic derivative is a deuterium labeled derivative (i.e., being enriched with ²H with regard to one or more atoms thereof). In some embodiments, the derivative is a ²H labeled derivative. In some embodiments, the derivative is a ¹³C labeled derivative or a ¹⁴C labeled derivative. In some embodiments, the derivative is a ¹⁸F labeled derivative. In some embodiments, the derivative is a ¹²³I labeled derivative, a ¹²⁴I labeled derivative, a ¹²⁵I labeled derivative, a ¹²⁹I labeled derivative, a ¹³¹I labeled derivative, a ¹³⁵I labeled derivative, or any combination thereof. In some embodiments, the derivative is a ³²P labeled derivative or a ³²P labeled derivative. In some embodiments, the derivative is a ³³S labeled derivative, a ³¹S labeled derivative, a ³⁵S labeled derivative, a ³⁶S labeled derivative, or any combination thereof.

It is also understood that isotopical substitution may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

It will be understood that while compounds disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a compound herein in a particular configuration intends to encompass, and to refer to, each of the available isomers, tautomers, regioisomers, and stereoisomers of the compound, or any mixture thereof; while the presentation further intends to refer to the specific configuration of the compound.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

As used herein, the term “isomerism” means compounds that have identical molecular formulae but differ in the sequence of bonding of their atoms or in the arrangement of their atoms in space. Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of any one of the Formulae include all such forms. For the avoidance of doubt, where a compound can exist in one of several tautomeric forms, and only one is specifically described or shown, all others are nevertheless embraced by the Formulae disclosed herein. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

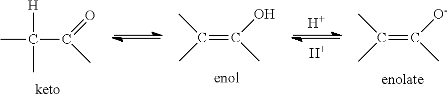

Compounds of any one of the Formulae disclosed herein containing an amine function may also form N-oxides. A reference herein to a compound of any one of the Formulae herein that contains an amine function also includes the N-oxide. Where a compound contains several amine functions, one or more than one nitrogen atom may be oxidized to form an N-oxide. Particular examples of N-oxides are the N-oxides of a tertiary amine or a nitrogen atom of a nitrogen-containing heterocycle. N-oxides can be formed by treatment of the corresponding amine with an oxidizing agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Conjugates Containing Nucleotide-Based Enhancement Units

As used herein, the term “conjugate” refers to a compound or complex that comprises a Nucleic Acid Agent being covalently attached to a ligand. In some embodiments, the conjugate further comprises a Nucleotide-Based Enhancement Unit described herein.

In some aspects, the present disclosure provides a conjugate or a pharmaceutically acceptable salt thereof, comprising:
(i) one or more Nucleic Acid Agent;
(ii) one or more Ligand; and
(iii) one or more Nucleotide-Based Enhancement Unit, wherein each Nucleotide-Based Enhancement Unit independently comprises from 2 to 30 nucleotides, wherein each nucleotide independently is of;

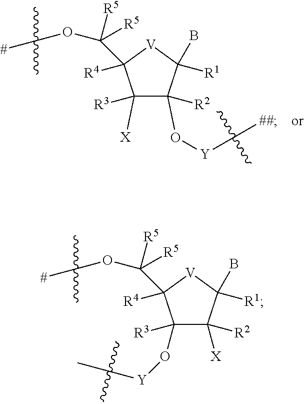

wherein:
variables B, V, X, Y, R¹, R², R³, R⁴, and R⁵ are described herein;
each # independently denotes an attachment to the 2′- or 3′-position of another nucleotide of the Nucleotide-Based Enhancement Unit, or denotes H or an attachment to the rest of the conjugate when the nucleotide is at the 5′-end of the Nucleotide-Based Enhancement Unit; and
each ## independently denotes an attachment to the 5′-position of another nucleotide of the Nucleotide-Based Enhancement Unit, or denotes H or an attachment to the rest of the conjugate when the nucleotide is at the 2′- or 3′-end of the Nucleotide-Based Enhancement Unit.

It is understood that the one or more Ligand and one or more Nucleotide-Based Enhancement Unit each independently is attached to a terminal position or an internal position of the one or more Nucleic Acid Agent. Further, it is understood that, when a Nucleic Acid Agent comprises more than one strand (e.g., a sense strand and an antisense strand), the one or more Ligand and one or more Nucleotide-Based Enhancement Unit may be attached to the same strand, or different strands, of the Nucleic Acid Agent.

In some embodiments, the conjugate further comprising one or more Linker Unit.

In some embodiments, the conjugate comprises a double strand RNA (e.g., double strand siRNA), one or more Ligand, and one or more Nucleotide-Based Enhancement Unit.

In some embodiments, at least one Nucleotide-Based Enhancement Unit is attached to the Nucleic Acid Agent (e.g., siRNA) directly.

In some embodiments, at least one Nucleotide-Based Enhancement Unit is attached to the Nucleic Acid Agent (e.g., siRNA) via a Linker Unit.

In some embodiments, at least one Nucleotide-Based Enhancement Unit is attached to the Nucleic Acid Agent (e.g., siRNA) directly or via a Linker Unit.

In some embodiments, at least one Nucleotide-Based Enhancement Unit is attached to a terminal position (e.g., a nucleotide at the 3′-end or the 5′-end) or internal position (e.g., a nucleotide that is not at the 3′-end or the 5′-end) of the Nucleic Acid Agent (e.g., siRNA) directly or via a Linker Unit.

In some embodiments, at least one Nucleotide-Based Enhancement Unit is attached to a terminal position (e.g., a nucleotide at the 3′-end or the 5′-end) of the Nucleic Acid Agent (e.g., siRNA) directly or via a Linker Unit.

In some embodiments, at least one Nucleotide-Based Enhancement Unit is attached to a nucleotide at the 3′-end of the Nucleic Acid Agent (e.g., siRNA) directly or via a Linker Unit.

In some embodiments, at least one Nucleotide-Based Enhancement Unit is attached to a nucleotide at the 5′-end of the Nucleic Acid Agent (e.g., siRNA) directly or via a Linker Unit.

In some embodiments, at least one Nucleotide-Based Enhancement Unit is attached to an internal position (e.g., a nucleotide that is not at the 3′-end or the 5′-end) of the Nucleic Acid Agent (e.g., siRNA) directly or via a Linker Unit.

In some embodiments, at least one Nucleotide-Based Enhancement Unit is attached to the sense or antisense strand of the Nucleic Acid Agent (e.g., siRNA) directly or via a Linker Unit.

Tn some embodiments, at least one Nucleotide-Based Enhancement Unit is attached to the sense strand of the Nucleic Acid Agent (e.g., siRNA) directly or via a Linker Unit.

Tn some embodiments, at least one Nucleotide-Based Enhancement Unit is attached to the sense strand of the Nucleic Acid Agent (e.g., siRNA) directly or via a Linker Unit, and the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to the antisense strand of the Nucleic Acid Agent (e.g., siRNA) directly or via a Linker Unit.

In some embodiments, at least one Nucleotide-Based Enhancement Unit is attached to the antisense strand of the Nucleic Acid Agent (e.g., siRNA) directly or via a Linker Unit, and the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to a terminal position (e.g., a nucleotide at the 3′-end or the 5′-end) of the sense strand of the Nucleic Acid Agent (e.g., siRNA) directly or via a Linker Unit.

In some embodiments, at least one Nucleotide-Based Enhancement Unit is attached to a terminal position (e.g., a nucleotide at the 3′-end or the 5′-end) of the sense strand of the Nucleic Acid Agent (e.g., siRNA) directly or via a Linker Unit, and the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to a nucleotide at the 3′-end of the sense strand of the Nucleic Acid Agent (e.g., siRNA) directly or via a Linker Unit.

In some embodiments, at least one Nucleotide-Based Enhancement Unit is attached to a nucleotide at the 3′-end of the sense strand of the Nucleic Acid Agent (e.g., siRNA) directly or via a Linker Unit, and the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to a nucleotide at the 5′-end of the sense strand of the Nucleic Acid Agent (e.g., siRNA) directly or via a Linker Unit.

Tn some embodiments, at least one Nucleotide-Based Enhancement Unit is attached to a nucleotide at the 5′-end of the sense strand of the Nucleic Acid Agent (e.g., siRNA) directly or via a Linker Unit, and the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to an internal position (e.g., a nucleotide that is not at the 3′-end or the 5′-end) of the sense strand of the Nucleic Acid Agent (e.g., siRNA) directly or via a Linker Unit.

In some embodiments, at least one Nucleotide-Based Enhancement Unit is attached to an internal position (e.g., a nucleotide that is not at the 3′-end or the 5′-end) of the sense strand of the Nucleic Acid Agent (e.g., siRNA) directly or via a Linker Unit, and the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to a terminal position (e.g., a nucleotide at the 3′-end or the 5′-end) of the antisense strand of the Nucleic Acid Agent (e.g., siRNA) directly or via a Linker Unit.

In some embodiments, at least one Nucleotide-Based Enhancement Unit is attached to a terminal position (e.g., a nucleotide at the 3′-end or the 5′-end) of the antisense strand of the Nucleic Acid Agent (e.g., siRNA) directly or via a Linker Unit, and the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to a nucleotide at the 3′-end of the antisense strand of the Nucleic Acid Agent (e.g., siRNA) directly or via a Linker Unit.

In some embodiments, at least one Nucleotide-Based Enhancement Unit is attached to a nucleotide at the 3′-end of the antisense strand of the Nucleic Acid Agent (e.g., siRNA) directly or via a Linker Unit, and the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to a nucleotide at the 5′-end of the antisense strand of the Nucleic Acid Agent (e.g., siRNA) directly or via a Linker Unit.

Tn some embodiments, at least one Nucleotide-Based Enhancement Unit is attached to a nucleotide at the 5′-end of the antisense strand of the Nucleic Acid Agent (e.g., siRNA) directly or via a Linker Unit, and the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to an internal position (e.g., a nucleotide that is not at the 3′-end or the 5′-end) of the antisense strand of the Nucleic Acid Agent (e.g., siRNA) directly or via a Linker Unit.

In some embodiments, at least one Nucleotide-Based Enhancement Unit is attached to an internal position (e.g., a nucleotide that is not at the 3′-end or the 5′-end) of the antisense strand of the Nucleic Acid Agent (e.g., siRNA) directly or via a Linker Unit, and the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to an overhang (e.g., 3′ or 5′ overhang) of the Nucleic Acid Agent (e.g., siRNA) directly or via a Linker Unit.

In some embodiments, at least one Nucleotide-Based Enhancement Unit is attached to the 3′ overhang of the Nucleic Acid Agent (e.g., siRNA) directly or via a Linker Unit.

In some embodiments, at least one Nucleotide-Based Enhancement Unit is attached to the 5′ overhang of the Nucleic Acid Agent (e.g., siRNA) directly or via a Linker Unit.

In some embodiments, at least one Nucleotide-Based Enhancement Unit is attached to an overhang (e.g., 3′ or 5′ overhang) of the antisense strand of the Nucleic Acid Agent (e.g., siRNA) directly or via a Linker Unit.

In some embodiments, at least one Nucleotide-Based Enhancement Unit is attached to the 3′ overhang of the antisense strand of the Nucleic Acid Agent (e.g., siRNA) directly or via a Linker Unit.

In some embodiments, at least one Nucleotide-Based Enhancement Unit is attached to the 5′ overhang of the antisense strand of the Nucleic Acid Agent (e.g., siRNA) directly or via a Linker Unit.

In some embodiments, at least one Nucleotide-Based Enhancement Unit is attached to an overhang (e.g., 3′ or 5′ overhang) of the sense strand of the Nucleic Acid Agent (e.g., siRNA) directly or via a Linker Unit.

In some embodiments, at least one Nucleotide-Based Enhancement Unit is attached to the 3′ overhang of the sense strand of the Nucleic Acid Agent (e.g., siRNA) directly or via a Linker Unit.

In some embodiments, at least one Nucleotide-Based Enhancement Unit is attached to the 5′ overhang of the sense strand of the Nucleic Acid Agent (e.g., siRNA) directly or via a Linker Unit.

In some embodiments, at least one Nucleotide-Based Enhancement Unit is attached to the sense strand of the Nucleic Acid Agent (e.g., siRNA) and does not bind to any part of the antisense strand of the Nucleic Acid Agent (e.g., siRNA).

In some embodiments, at least one Nucleotide-Based Enhancement Unit is attached to the antisense strand of the Nucleic Acid Agent (e.g., siRNA) and does not bind to any part of the sense strand of the Nucleic Acid Agent (e.g., siRNA).

In some embodiments, at least one Nucleotide-Based Enhancement Unit does not bind to any part of the antisense strand of the Nucleic Acid Agent (e.g., siRNA).

In some embodiments, at least one Nucleotide-Based Enhancement Unit does not bind to any part of the sense strand of the Nucleic Acid Agent (e.g., siRNA).

In some embodiments, at least one Nucleotide-Based Enhancement Unit binds to the antisense strand of the Nucleic Acid Agent (e.g., siRNA).

In some embodiments, at least one Nucleotide-Based Enhancement Unit binds to the sense strand of the Nucleic Acid Agent (e.g., siRNA).

In some embodiments, at least one Nucleotide-Based Enhancement Unit is attached to a terminal position (e.g., a nucleotide at the 3′-end or the 5′-end) or internal position (e.g., a nucleotide that is not at the 3′-end or the 5′-end) of the sense or antisense strand of the Nucleic Acid Agent (e.g., siRNA) directly or via a Linker Unit.

In some embodiments, at least one Nucleotide-Based Enhancement Unit is attached to the Nucleic Acid Agent (e.g., siRNA) directly or via a Linker Unit, and is attached to the Ligand directly or via a Linker Unit.

In some embodiments, at least one Nucleotide-Based Enhancement Unit is attached to a terminal position (e.g., a nucleotide at the 3′-end or the 5′-end) or internal position (e.g., a nucleotide that is not at the 3′-end or the 5′-end) of the Nucleic Acid Agent (e.g., siRNA) directly or via a Linker Unit, and is attached to the Ligand directly or via a Linker Unit.

Tn some embodiments, at least one Nucleotide-Based Enhancement Unit is attached to a terminal position (e.g., a nucleotide at the 3′-end or the 5′-end) of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a nucleotide at the 3′-end of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a nucleotide at the 5′-end of the Nucleic Acid Agent (e.g., siRNA) directly or via a Linker Unit, and is attached to the Ligand directly or via a Linker Unit.

In some embodiments, at least one Nucleotide-Based Enhancement Unit is attached to an internal position (e.g., a nucleotide that is not at the 3′-end or the 5′-end) of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the sense or antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the sense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the sense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to the antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the antisense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to a terminal position (e.g., a nucleotide at the 3′-end or the 5′-end) of the sense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a terminal position (e.g., a nucleotide at the 3′-end or the 5′-end) of the sense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to a nucleotide at the 3′-end of the sense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a nucleotide at the 3′-end of the sense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to a nucleotide at the 5′-end of the sense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a nucleotide at the 5′-end of the sense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to an internal position (e.g., a nucleotide that is not at the 3′-end or the 5′-end) of the sense strand of the Nucleic Acid Agent (e.g., siRNA) directly or via a Linker Unit, and is attached to the Ligand directly or via a Linker Unit.

Tn some embodiments, at least one Nucleotide-Based Enhancement Unit is attached to an internal position (e.g., a nucleotide that is not at the 3′-end or the 5′-end) of the sense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the antisense strand of the Nucleic Acid Agent (e.g., siRNA).

In some embodiments, at least one Nucleotide-Based Enhancement Unit is attached to a terminal position (e.g., a nucleotide at the 3′-end or the 5′-end) of the antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a terminal position (e.g., a nucleotide at the 3′-end or the 5′-end) of the antisense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to a nucleotide at the 3′-end of the antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a nucleotide at the 3′-end of the antisense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to a nucleotide at the 5′-end of the antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to a nucleotide at the 5′-end of the antisense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to an internal position (e.g., a nucleotide that is not at the 3′-end or the 5′-end) of the antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to an internal position (e.g., a nucleotide that is not at the 3′-end or the 5′-end) of the antisense strand of the Nucleic Acid Agent (e.g., siRNA) directly or via a Linker Unit, and is attached to the Ligand directly or via a Linker Unit, wherein the Nucleotide-Based Enhancement Unit does not interact with (e.g., bind to) any portion of the sense strand of the Nucleic Acid Agent (e.g., siRNA).

In some embodiments, at least one Nucleotide-Based Enhancement Unit is attached to an overhang (e.g., 3′ or 5′ overhang) of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the 3′ overhang of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the 5′ overhang of the Nucleic Acid Agent (e.g., siRNA) directly or via a Linker Unit, and is attached to the Ligand directly or via a Linker Unit.

In some embodiments, at least one Nucleotide-Based Enhancement Unit is attached to an overhang (e.g., 3′ or 5′ overhang) of the antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the 3′ overhang of the antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the 5′ overhang of the antisense strand of the Nucleic Acid Agent (e.g., siRNA) directly or via a Linker Unit, and is attached to the Ligand directly or via a Linker Unit.

Tn some embodiments, at least one Nucleotide-Based Enhancement Unit is attached to an overhang (e.g., 3′ or 5′ overhang) of the sense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the 3′ overhang of the sense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the 5′ overhang of the sense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the sense strand of the Nucleic Acid Agent (e.g., siRNA) and does not bind to any part of the antisense strand of the Nucleic Acid Agent (e.g., siRNA), and is attached to the Ligand directly or via a Linker Unit.

In some embodiments, at least one Nucleotide-Based Enhancement Unit is attached to the antisense strand of the Nucleic Acid Agent (e.g., siRNA) and does not bind to any part of the sense strand of the Nucleic Acid Agent (e.g., siRNA), and is attached to the Ligand directly or via a Linker Unit.

In some embodiments, at least one Nucleotide-Based Enhancement Unit does not bind to any part of the antisense strand of the Nucleic Acid Agent (e.g., siRNA), and is attached to the Ligand directly or via a Linker Unit.

In some embodiments, at least one Nucleotide-Based Enhancement Unit does not bind to any part of the sense strand of the Nucleic Acid Agent (e.g., siRNA), and is attached to the Ligand directly or via a Linker Unit.

In some embodiments, at least one Nucleotide-Based Enhancement Unit binds to the antisense strand of the Nucleic Acid Agent (e.g., siRNA), and is attached to the Ligand directly or via a Linker Unit.

In some embodiments, at least one Nucleotide-Based Enhancement Unit binds to the sense strand of the Nucleic Acid Agent (e.g., siRNA), and is attached to the Ligand directly or via a Linker Unit.

In some embodiments, at least one Nucleotide-Based Enhancement Unit is attached to a terminal position (e.g., a nucleotide at the 3′-end or the 5′-end) or internal position (e.g., a nucleotide that is not at the 3′-end or the 5′-end) of the sense or antisense strand of the Nucleic Acid Agent (e.g., siRNA) directly or via a Linker Unit, and is attached to the Ligand directly or via a Linker Unit.

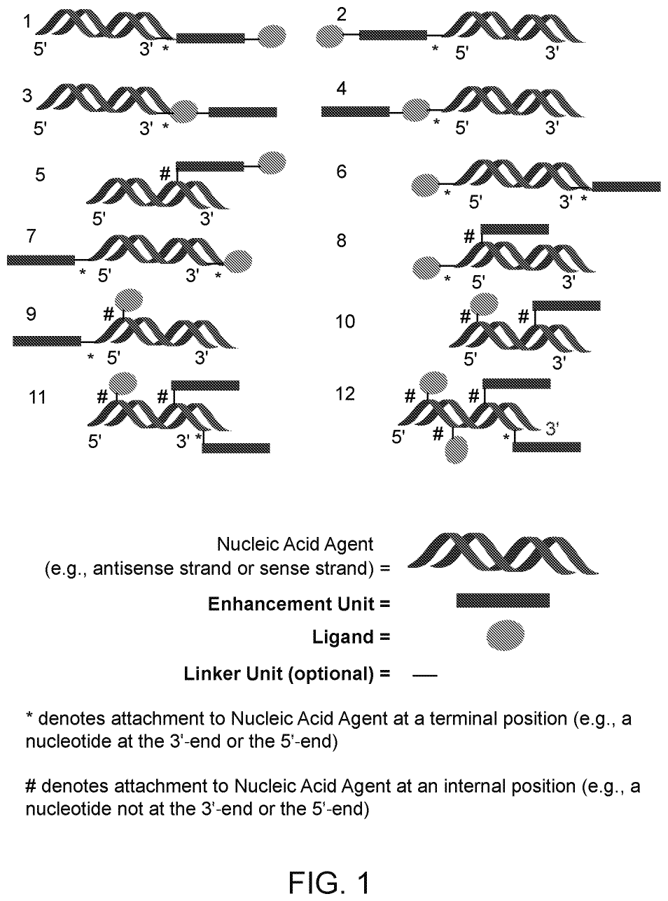

In some embodiments, at least one Nucleotide-Based Enhancement Unit is attached to a terminal position (e.g., a nucleotide at the 3′-end or the 5′-end) or internal position (e.g., a nucleotide that is not at the 3′-end or the 5′-end) of the sense or antisense strand of the Nucleic Acid Agent (e.g., siRNA) directly or via a Linker Unit, and is attached to the Ligand directly or via a Linker Unit, as described in FIG. 1.

In some embodiments, at least one Nucleotide-Based Enhancement Unit is attached to a terminal position (e.g., a nucleotide at the 3′-end or the 5′-end) of the sense or antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to an internal position (e.g., a nucleotide that is not at the 3′-end or the 5′-end) of the sense or antisense strand of the Nucleic Acid Agent (e.g., siRNA) directly or via a Linker Unit, and is attached to the Ligand directly or via a Linker Unit.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 1 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 2 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 3 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 4 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 5 of FIG. 1.

Tn some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 6 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 7 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 8 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 9 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 10 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 11 of FIG. 1.

In some embodiments, at least one Nucleotide-Based Enhancement Unit is attached to the Nucleic Acid Agent (e.g., siRNA) directly or via a Linker Unit, and is attached to the Ligand directly or via a Linker Unit, as described in Conjugate No. 12 of FIG. 1.

In some embodiments, at least one Ligand is attached to the Nucleic Acid Agent (e.g., siRNA) directly.

In some embodiments, at least one Ligand is attached to the Nucleic Acid Agent (e.g., siRNA) via a Linker Unit.

In some embodiments, at least one Ligand is attached to the Nucleic Acid Agent (e.g., siRNA) via a Nucleotide-Based Enhancement Unit.

Tn some embodiments, at least one Ligand is attached to Nucleotide-Based Enhancement Unit directly.

Tn some embodiments, at least one Ligand is attached to Nucleotide-Based Enhancement Unit via a Linker Unit.

In some embodiments, at least one Ligand is attached to the Nucleic Acid Agent (e.g., siRNA) via a Nucleotide-Based Enhancement Unit and a Linker Unit.

In some embodiments, the conjugates comprises:
(Nucleic Acid Agent)-[(Linker Unit)₀₋₁-(Nucleotide-Based Enhancement Unit)-(Linker Unit)₀₋₁-(Ligand)]₁₋₃;
(Nucleic Acid Agent)-[(Linker Unit)₀₋₁-(Ligand)-(Linker Unit)₀₋₁-(Nucleotide-Based Enhancement Unit)]₁₋₃; or
[(Nucleotide-Based Enhancement Unit)-(Linker Unit)₀₋₁]₁₋₃-(Nucleic Acid Agent)-[(Linker Unit)₀₋₁-(Ligand)]₁₋₃;
wherein each of the Linker Unit, Nucleotide-Based Enhancement Unit, and Ligand, when attached to the Nucleic Acid Agent (e.g., siRNA), independently is attached to a terminal position (e.g., a nucleotide at the 3′-end or the 5′-end) or an internal position (e.g., a nucleotide that is not at the 3′-end or the 5′-end) of the Nucleic Acid Agent (e.g., siRNA).

In some embodiments, the conjugate is selected from the conjugates described in Table D, wherein the Nucleic Acid Agent may optionally be further attached to one or more Linker Units, one or more Nucleotide-Based Enhancement Units, and/or one or more Ligands, at one or more of the internal positions of the Nucleic Acid Agent according to the attachment described herein.

TABLE D

| No. | Structure |
|---|---|
| D-1 | 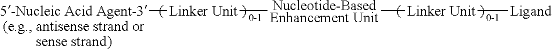 |
| D-2 | 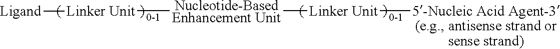 |
| D-3 | 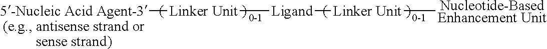 |
| D-4 | 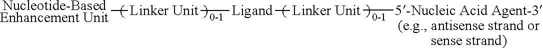 |
| D-5 | 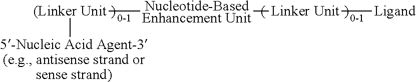 |
| D-6 | 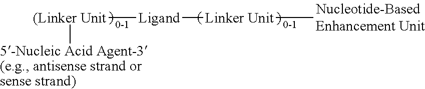 |
| D-7 | 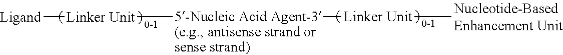 |
| D-8 | 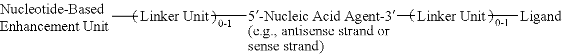 |
| D-9 | 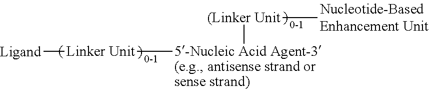 |
| D-10 | 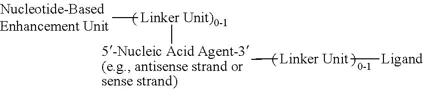 |
| D-11 | 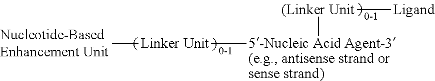 |
| D-12 | 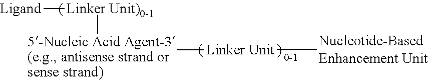 |
| D-13 | 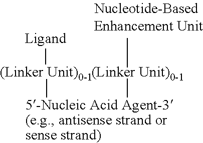 |
| D-14 | 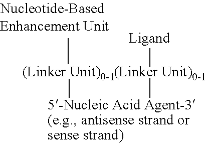 |
| D-15 | 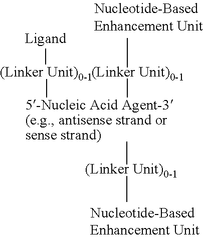 |
| D-16 | 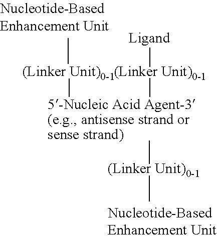 |
| D-17 | 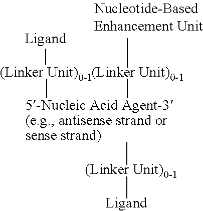 |
| D-18 | 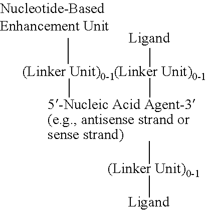 |
| D-19 | 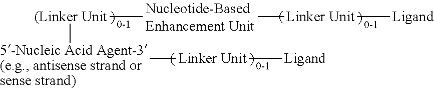 |
| D-20 | 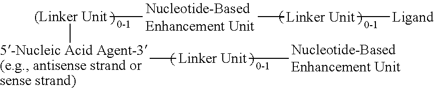 |
| D-21 | 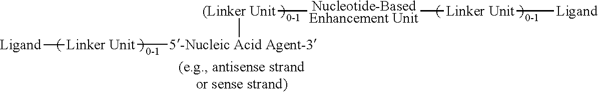 |
| D-22 | 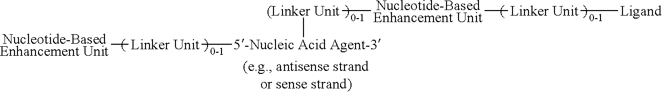 |
| D-23 | 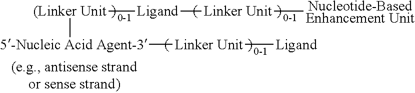 |
| D-24 | 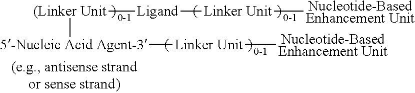 |
| D-25 | 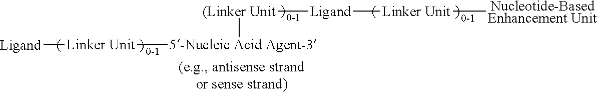 |
| D-26 | 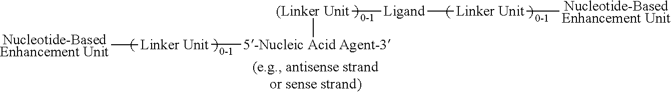 |
| D-27 | 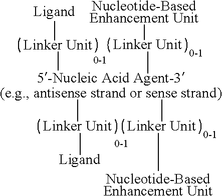 |
| D-28 | 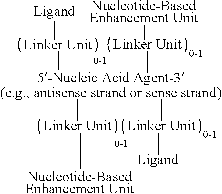 |
| D-29 | 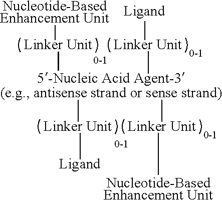 |
| D-30 | 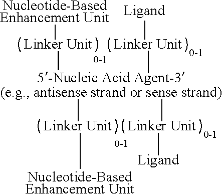 |
| D-31 | 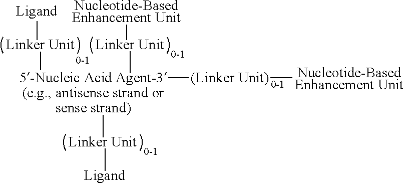 |
| D-32 | 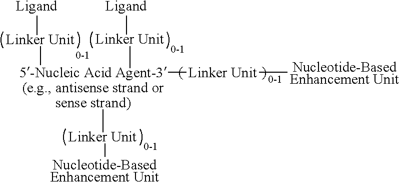 |
| D-33 | 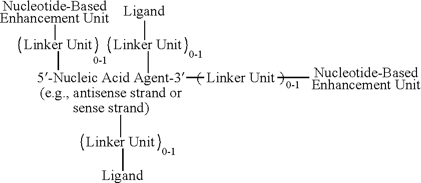 |
| D-34 | 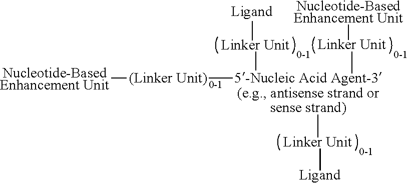 |
| D-35 | 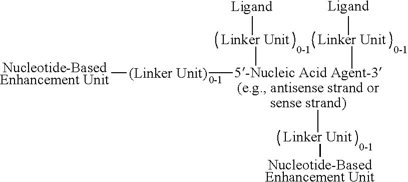 |
| D-36 | 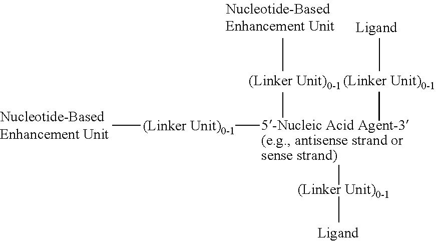 |
| D-37 | 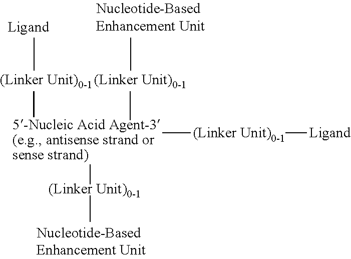 |
| D-38 | 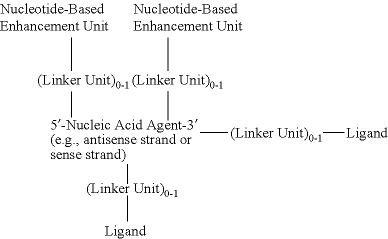 |
| D-39 | 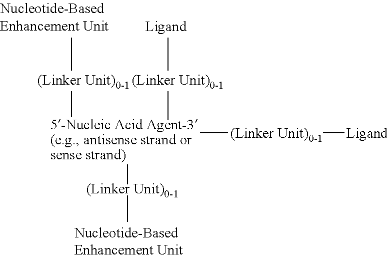 |
| D-40 | 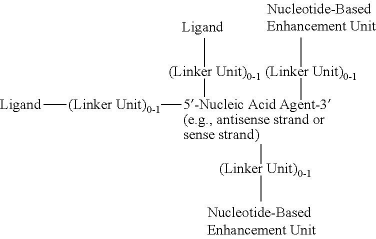 |
| D-41 | 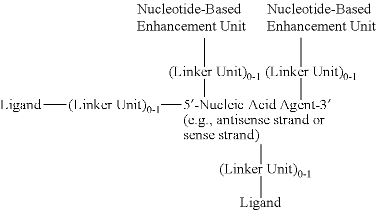 |
| D-42 | 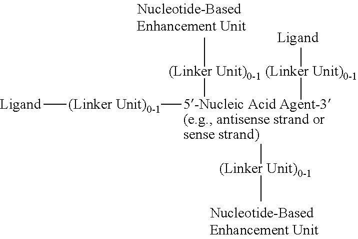 |
| D-43 | 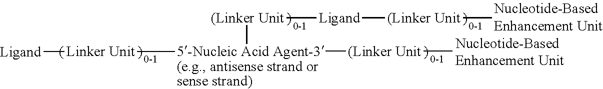 |
| D-44 | 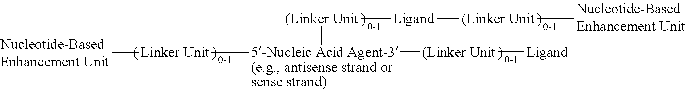 |
| D-45 | 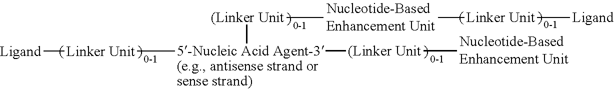 |
| D-46 | 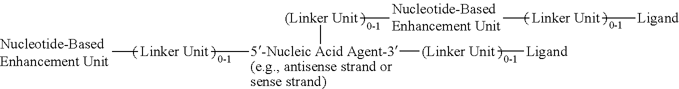 |
| D-47 | 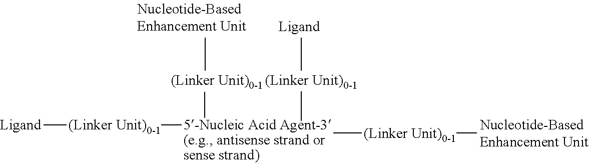 |
| D-48 | 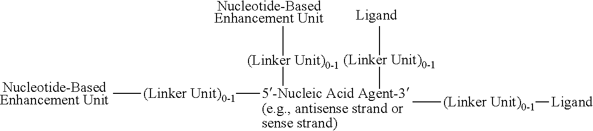 |
| D-49 | 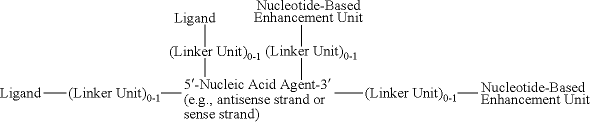 |
| D-50 | 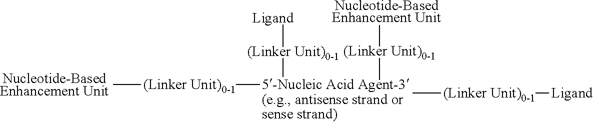 |

In it understood that the one or more attachments to the Nucleic Acid Agent may be at one or more internal positions (e.g.,

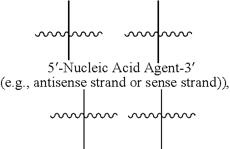

at the 3′-end position (e.g.,

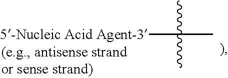

or at the 5′-end position (e.g.,

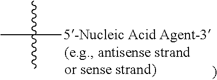

### of the Nucleic Acid Agent.

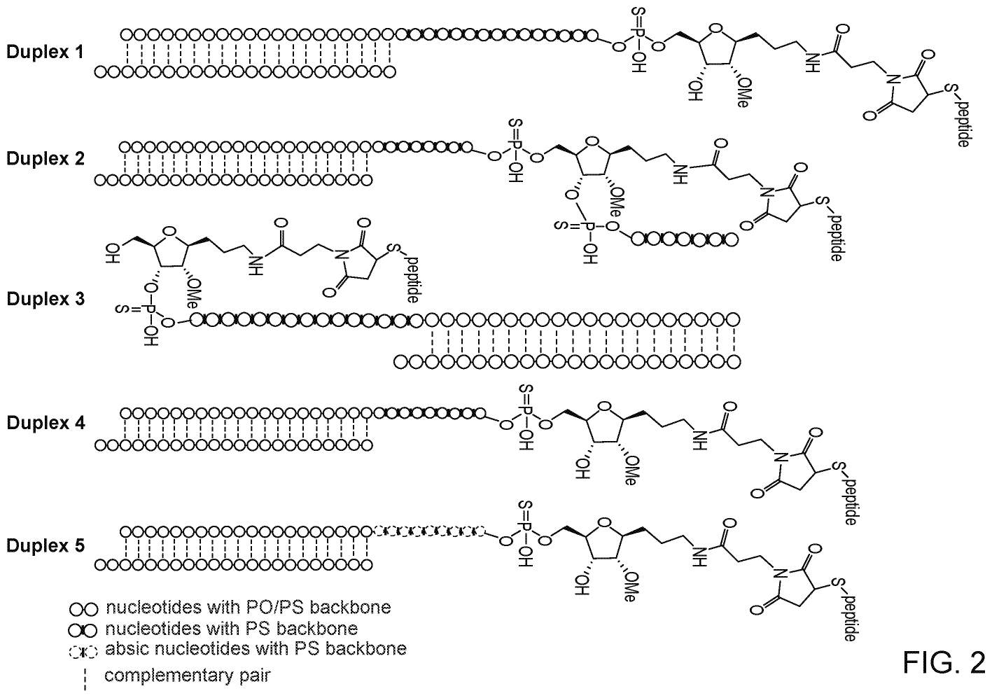

In some embodiments, the conjugate is selected from the conjugates described in FIGS. 1 and 2.

In some embodiments, the conjugate is selected from the conjugates described in FIG. 1.

In some embodiments, the conjugate is Conjugate No. 1 described in FIG. 1.

In some embodiments, the conjugate is Conjugate No. 1 described in FIG. 1, wherein the Nucleotide-Based Enhancement Unit is attached to the antisense strand of the Nucleic Acid Agent.

In some embodiments, the conjugate is Conjugate No. 1 described in FIG. 1, wherein the Nucleotide-Based Enhancement Unit is attached to the sense strand of the Nucleic Acid Agent.

In some embodiments, the conjugate is Conjugate No. 1 described in FIG. 1, wherein the Nucleotide-Based Enhancement Unit is attached to the 3′-end of the antisense strand of the Nucleic Acid Agent directly or via a Linker Unit, and is attached to the Ligand directly or via a Linker Unit.

In some embodiments, the conjugate is Conjugate No. 1 described in FIG. 1, wherein the Nucleotide-Based Enhancement Unit is attached to the 3′-end of the sense strand of the Nucleic Acid Agent directly or via a Linker Unit, and is attached to the Ligand directly or via a Linker Unit.

In some embodiments, the conjugate is Conjugate No. 2 described in FIG. 1.

Tn some embodiments, the conjugate is Conjugate No. 2 described in FIG. 1, wherein the Nucleotide-Based Enhancement Unit is attached to the antisense strand of the Nucleic Acid Agent.

In some embodiments, the conjugate is Conjugate No. 2 described in FIG. 1, wherein the Nucleotide-Based Enhancement Unit is attached to the sense strand of the Nucleic Acid Agent.

In some embodiments, the conjugate is Conjugate No. 2 described in FIG. 1, wherein the Nucleotide-Based Enhancement Unit is attached to the 5′-end of the antisense strand of the Nucleic Acid Agent directly or via a Linker Unit, and is attached to the Ligand directly or via a Linker Unit.

In some embodiments, the conjugate is Conjugate No. 2 described in FIG. 1, wherein the Nucleotide-Based Enhancement Unit is attached to the 5′-end of the sense strand of the Nucleic Acid Agent directly or via a Linker Unit, and is attached to the Ligand directly or via a Linker Unit.

In some embodiments, the conjugate is Conjugate No. 3 described in FIG. 1.

In some embodiments, the conjugate is Conjugate No. 3 described in FIG. 1, wherein the Nucleotide-Based Enhancement Unit is attached to the antisense strand of the Nucleic Acid Agent.

In some embodiments, the conjugate is Conjugate No. 3 described in FIG. 1, wherein the Nucleotide-Based Enhancement Unit is attached to the sense strand of the Nucleic Acid Agent.

In some embodiments, the conjugate is Conjugate No. 3 described in FIG. 1, wherein the Ligand is attached to the 3′-end of the antisense strand of the Nucleic Acid Agent directly or via a Linker Unit, and is attached to the Nucleotide-Based Enhancement Unit directly or via a Linker Unit.

In some embodiments, the conjugate is Conjugate No. 3 described in FIG. 1, wherein the Ligand is attached to the 3′-end of the sense strand of the Nucleic Acid Agent directly or via a Linker Unit, and is attached to the Nucleotide-Based Enhancement Unit directly or via a Linker Unit.

In some embodiments, the conjugate is Conjugate No. 4 described in FIG. 1.

In some embodiments, the conjugate is Conjugate No. 4 described in FIG. 1, wherein the Nucleotide-Based Enhancement Unit is attached to the antisense strand of the Nucleic Acid Agent.

In some embodiments, the conjugate is Conjugate No. 4 described in FIG. 1, wherein the Nucleotide-Based Enhancement Unit is attached to the sense strand of the Nucleic Acid Agent.

In some embodiments, the conjugate is Conjugate No. 4 described in FIG. 1, wherein the Ligand is attached to the 5′-end of the antisense strand of the Nucleic Acid Agent directly or via a Linker Unit, and is attached to the Nucleotide-Based Enhancement Unit directly or via a Linker Unit.

In some embodiments, the conjugate is Conjugate No. 4 described in FIG. 1, wherein the Ligand is attached to the 5′-end of the sense strand of the Nucleic Acid Agent directly or via a Linker Unit, and is attached to the Nucleotide-Based Enhancement Unit directly or via a Linker Unit.

In some embodiments, the conjugate is Conjugate No. 5 described in FIG. 1.

In some embodiments, the conjugate is Conjugate No. 5 described in FIG. 1, wherein the Nucleotide-Based Enhancement Unit is attached to the antisense strand of the Nucleic Acid Agent.

In some embodiments, the conjugate is Conjugate No. 5 described in FIG. 1, wherein the Nucleotide-Based Enhancement Unit is attached to the sense strand of the Nucleic Acid Agent.

In some embodiments, the conjugate is Conjugate No. 5 described in FIG. 1, wherein the Nucleotide-Based Enhancement Unit is attached to an internal position of the antisense strand of the Nucleic Acid Agent directly or via a Linker Unit, and is attached to the Ligand directly or via a Linker Unit.

In some embodiments, the conjugate is Conjugate No. 5 described in FIG. 1, wherein the Nucleotide-Based Enhancement Unit is attached to an internal position of the sense strand of the Nucleic Acid Agent directly or via a Linker Unit, and is attached to the Ligand directly or via a Linker Unit.

In some embodiments, the conjugate is Conjugate No. 6 described in FIG. 1.

In some embodiments, the conjugate is Conjugate No. 6 described in FIG. 1, wherein the Nucleotide-Based Enhancement Unit is attached to the antisense strand of the Nucleic Acid Agent.

In some embodiments, the conjugate is Conjugate No. 6 described in FIG. 1, wherein the Nucleotide-Based Enhancement Unit is attached to the sense strand of the Nucleic Acid Agent.

In some embodiments, the conjugate is Conjugate No. 6 described in FIG. 1, wherein the Nucleotide-Based Enhancement Unit is attached to 3′-end of the antisense strand of the Nucleic Acid Agent directly or via a Linker Unit, and the Ligand is attached to the 5′-end of the antisense strand of the Nucleic Acid Agent directly or via a Linker Unit.

In some embodiments, the conjugate is Conjugate No. 6 described in FIG. 1, wherein the Nucleotide-Based Enhancement Unit is attached to 3′-end of the antisense strand of the Nucleic Acid Agent directly or via a Linker Unit, and the Ligand is attached to the 5′-end of the sense strand of the Nucleic Acid Agent directly or via a Linker Unit.

In some embodiments, the conjugate is Conjugate No. 6 described in FIG. 1, wherein the Nucleotide-Based Enhancement Unit is attached to 3′-end of the sense strand of the Nucleic Acid Agent directly or via a Linker Unit, and the Ligand is attached to the 5′-end of the antisense strand of the Nucleic Acid Agent directly or via a Linker Unit.

In some embodiments, the conjugate is Conjugate No. 6 described in FIG. 1, wherein the Nucleotide-Based Enhancement Unit is attached to 3′-end of the sense strand of the Nucleic Acid Agent directly or via a Linker Unit, and the Ligand is attached to the 5′-end of the sense strand of the Nucleic Acid Agent directly or via a Linker Unit.

In some embodiments, the conjugate is Conjugate No. 7 described in FIG. 1.

In some embodiments, the conjugate is Conjugate No. 7 described in FIG. 1, wherein the Nucleotide-Based Enhancement Unit is attached to the antisense strand of the Nucleic Acid Agent.

In some embodiments, the conjugate is Conjugate No. 7 described in FIG. 1, wherein the Nucleotide-Based Enhancement Unit is attached to the sense strand of the Nucleic Acid Agent.

In some embodiments, the conjugate is Conjugate No. 7 described in FIG. 1, wherein the Nucleotide-Based Enhancement Unit is attached to 5′-end of the antisense strand of the Nucleic Acid Agent directly or via a Linker Unit, and the Ligand is attached to the 3′-end of the antisense strand of the Nucleic Acid Agent directly or via a Linker Unit.

In some embodiments, the conjugate is Conjugate No. 7 described in FIG. 1, wherein the Nucleotide-Based Enhancement Unit is attached to 5′-end of the sense strand of the Nucleic Acid Agent directly or via a Linker Unit, and the Ligand is attached to the 3′-end of the antisense strand of the Nucleic Acid Agent directly or via a Linker Unit.

In some embodiments, the conjugate is Conjugate No. 7 described in FIG. 1, wherein the Nucleotide-Based Enhancement Unit is attached to 5′-end of the antisense strand of the Nucleic Acid Agent directly or via a Linker Unit, and the Ligand is attached to the 3′-end of the sense strand of the Nucleic Acid Agent directly or via a Linker Unit.

In some embodiments, the conjugate is Conjugate No. 7 described in FIG. 1, wherein the Nucleotide-Based Enhancement Unit is attached to 5′-end of the sense strand of the Nucleic Acid Agent directly or via a Linker Unit, and the Ligand is attached to the 3′-end of the sense strand of the Nucleic Acid Agent directly or via a Linker Unit.

In some embodiments, the conjugate is Conjugate No. 8 described in FIG. 1.

In some embodiments, the conjugate is Conjugate No. 8 described in FIG. 1, wherein the Nucleotide-Based Enhancement Unit is attached to the antisense strand of the Nucleic Acid Agent.

In some embodiments, the conjugate is Conjugate No. 8 described in FIG. 1, wherein the Nucleotide-Based Enhancement Unit is attached to the sense strand of the Nucleic Acid Agent.

In some embodiments, the conjugate is Conjugate No. 8 described in FIG. 1, wherein the Nucleotide-Based Enhancement Unit is attached to an internal position of the antisense strand of the Nucleic Acid Agent directly or via a Linker Unit, and the Ligand is attached to the 5′-end of the antisense strand of the Nucleic Acid Agent directly or via a Linker Unit.

In some embodiments, the conjugate is Conjugate No. 8 described in FIG. 1, wherein the Nucleotide-Based Enhancement Unit is attached to an internal position of the sense strand of the Nucleic Acid Agent directly or via a Linker Unit, and the Ligand is attached to the 5′-end of the antisense strand of the Nucleic Acid Agent directly or via a Linker Unit.

In some embodiments, the conjugate is Conjugate No. 8 described in FIG. 1, wherein the Nucleotide-Based Enhancement Unit is attached to an internal position of the antisense strand of the Nucleic Acid Agent directly or via a Linker Unit, and the Ligand is attached to the 5′-end of the sense strand of the Nucleic Acid Agent directly or via a Linker Unit.

In some embodiments, the conjugate is Conjugate No. 8 described in FIG. 1, wherein the Nucleotide-Based Enhancement Unit is attached to an internal position of the sense strand of the Nucleic Acid Agent directly or via a Linker Unit, and the Ligand is attached to the 5′-end of the sense strand of the Nucleic Acid Agent directly or via a Linker Unit.

In some embodiments, the conjugate is Conjugate No. 9 described in FIG. 1.

In some embodiments, the conjugate is Conjugate No. 9 described in FIG. 1, wherein the Nucleotide-Based Enhancement Unit is attached to the antisense strand of the Nucleic Acid Agent.

In some embodiments, the conjugate is Conjugate No. 9 described in FIG. 1, wherein the Nucleotide-Based Enhancement Unit is attached to the sense strand of the Nucleic Acid Agent.

In some embodiments, the conjugate is Conjugate No. 9 described in FIG. 1, wherein the Ligand is attached to an internal position of the antisense strand of the Nucleic Acid Agent directly or via a Linker Unit, and the Nucleotide-Based Enhancement Unit is attached to the 5′-end of the antisense strand of the Nucleic Acid Agent directly or via a Linker Unit.

In some embodiments, the conjugate is Conjugate No. 9 described in FIG. 1, wherein the Ligand is attached to an internal position of the sense strand of the Nucleic Acid Agent directly or via a Linker Unit, and the Nucleotide-Based Enhancement Unit is attached to the 5′-end of the antisense strand of the Nucleic Acid Agent directly or via a Linker Unit.

In some embodiments, the conjugate is Conjugate No. 9 described in FIG. 1, wherein the Ligand is attached to an internal position of the antisense strand of the Nucleic Acid Agent directly or via a Linker Unit, and the Nucleotide-Based Enhancement Unit is attached to the 5′-end of the sense strand of the Nucleic Acid Agent directly or via a Linker Unit.

In some embodiments, the conjugate is Conjugate No. 9 described in FIG. 1, wherein the Ligand is attached to an internal position of the sense strand of the Nucleic Acid Agent directly or via a Linker Unit, and the Nucleotide-Based Enhancement Unit is attached to the 5′-end of the sense strand of the Nucleic Acid Agent directly or via a Linker Unit.

In some embodiments, the conjugate is Conjugate No. 10 described in FIG. 1.

Tn some embodiments, the conjugate is Conjugate No. 10 described in FIG. 1, wherein the Nucleotide-Based Enhancement Unit is attached to the antisense strand of the Nucleic Acid Agent.

In some embodiments, the conjugate is Conjugate No. 10 described in FIG. 1, wherein the Nucleotide-Based Enhancement Unit is attached to the sense strand of the Nucleic Acid Agent.

In some embodiments, the conjugate is Conjugate No. 10 described in FIG. 1, wherein the Ligand is attached to an internal position of the antisense strand of the Nucleic Acid Agent directly or via a Linker Unit, and the Nucleotide-Based Enhancement Unit is attached to an internal position of the antisense strand of the Nucleic Acid Agent directly or via a Linker Unit.

In some embodiments, the conjugate is Conjugate No. 10 described in FIG. 1, wherein the Ligand is attached to an internal position of the sense strand of the Nucleic Acid Agent directly or via a Linker Unit, and the Nucleotide-Based Enhancement Unit is attached to an internal position of the antisense strand of the Nucleic Acid Agent directly or via a Linker Unit.

In some embodiments, the conjugate is Conjugate No. 10 described in FIG. 1, wherein the Ligand is attached to an internal position of the antisense strand of the Nucleic Acid Agent directly or via a Linker Unit, and the Nucleotide-Based Enhancement Unit is attached to an internal position of the sense strand of the Nucleic Acid Agent directly or via a Linker Unit.

In some embodiments, the conjugate is Conjugate No. 10 described in FIG. 1, wherein the Ligand is attached to an internal position of the sense strand of the Nucleic Acid Agent directly or via a Linker Unit, and the Nucleotide-Based Enhancement Unit is attached to an internal position of the sense strand of the Nucleic Acid Agent directly or via a Linker Unit.

In some embodiments, the conjugate is Conjugate No. 11 described in FIG. 1.

In some embodiments, the conjugate is Conjugate No. 11 described in FIG. 1, wherein the Nucleotide-Based Enhancement Unit is attached to the antisense strand of the Nucleic Acid Agent.

In some embodiments, the conjugate is Conjugate No. 11 described in FIG. 1, wherein the Nucleotide-Based Enhancement Unit is attached to the sense strand of the Nucleic Acid Agent.

Tn some embodiments, the conjugate is Conjugate No. 11 described in FIG. 1, wherein the Ligand is attached to an internal position of the antisense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 11 described in FIG. 1, wherein the Ligand is attached to an internal position of the sense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 11 described in FIG. 1, wherein the Ligand is attached to an internal position of the antisense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 11 described in FIG. 1, wherein the Ligand is attached to an internal position of the antisense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 11 described in FIG. 1, wherein the Ligand is attached to an internal position of the sense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 11 described in FIG. 1, wherein the Ligand is attached to an internal position of the antisense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 11 described in FIG. 1, wherein the Ligand is attached to an internal position of the sense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 11 described in FIG. 1, wherein the Ligand is attached to an internal position of the sense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 12 described in FIG. 1.

In some embodiments, the conjugate is Conjugate No. 12 described in FIG. 1, wherein the Nucleotide-Based Enhancement Unit is attached to the antisense strand of the Nucleic Acid Agent.

In some embodiments, the conjugate is Conjugate No. 12 described in FIG. 1, wherein the Nucleotide-Based Enhancement Unit is attached to the sense strand of the Nucleic Acid Agent.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the sense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the sense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 12 described in FIG. 1, wherein one Ligand is attached to an internal position of the sense strand of the Nucleic Acid Agent directly or via a Linker Unit, another Ligand is attached to an internal position of the sense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the sense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 12 described in FIG. 1, wherein one Ligand is attached to an internal position of the sense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the antisense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 12 described in FIG. 1, wherein one Ligand is attached to an internal position of the sense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the sense strand of the Nucleic Acid Agent directly or via a Linker Unit and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the sense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the sense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the antisense strand of the Nucleic Acid Agent directly or via a Linker Unit.

In some embodiments, the conjugate is Conjugate No. 12 described in FIG. 1, wherein one Ligand is attached to an internal position of the antisense strand of the Nucleic Acid Agent directly or via a Linker Unit, another Ligand is attached to an internal position of the antisense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is Conjugate No. 12 described in FIG. 1, wherein one Ligand is attached to an internal position of the sense strand of the Nucleic Acid Agent directly or via a Linker Unit, another Ligand is attached to an internal position of the sense strand of the Nucleic Acid Agent directly or via a Linker Unit, one Nucleotide-Based Enhancement Unit is attached to an internal position of the sense strand of the Nucleic Acid Agent directly or via a Linker Unit, and another Nucleotide-Based Enhancement Unit is attached to the 3′-end of the sense strand of the Nucleic Acid Agent directly or via a Linker Unit.

In some embodiments, the conjugate is selected from the conjugates described in FIG. 2.

In some embodiments, the conjugate is selected from the structures described in Table C, wherein
attachments to [Nucleic Acid Agent](e.g., the antisense strand or the sense strand of the Nucleic Acid Agent) can independently be at a terminal position (e.g., a nucleotide at the 3′-end or the 5′-end) or internal position (e.g., a nucleotide that is not at the 3′-end or the 5′-end) of the Nucleic Acid Agent; and
attachments to [Nucleic Acid Agent] and [Ligand] can independently be direct or via a Linker Unit.

TABLE C

| No. | Structure |
|---|---|
| C-1 | [Nucleic Acid Agent]-[[mGs]-[mCs]-[mAs]-[mGs]-[mCs]-[mCs]-[mGs]-[mAs]- |
| | [mAs]-[mAs]-[mCs]-[mCs]-[mGs]-[mAs]-[mCs] (SEQ ID NO: 1)]-[Ligand] |
| C-2 | [Nucleic Acid Agent]-[mGs]-[mCs]-[mAs]-[mGs]-[mCs]-[mCs]-[mGs]-[mAs]-[ |
| | Ligand]-[mAs]-[mAs]-[mCs]-[mCs]-[mGs]-[mAs]-[mC] |
| C-3 | [Ligand]-[[mGs]-[mCs]-[mAs]-[mGs]-[mCs]-[mCs]-[mGs]-[mAs]-[mAs]-[mAs]- |
| | [mCs]-[mCs]-[mGs]-[mAs]-[mCs] (SEQ ID NO: 1)]-[Nucleic Acid Agent] |
| C-4 | [Nucleic Acid Agent]-[mGs]-[mCs]-[mAs]-[mGs]-[mCs]-[mCs]-[mGs]-[mAs]- |
| | [mAs]-[Ligand] |
| C-5 | [Nucleic Acid Agent]-[Abs]-[Abs]-[Abs]-[Abs]-[Abs]-[Abs]-[Abs]-[Abs]-[Abs]- |
| | [Ligand] |
| C-6 | [Nucleic Acid Agent]-[mGs]-[mCs]-[mAs]-[mGs]-[mCs]-[mCs]-[Ligand] |
| C-7 | [Nucleic Acid Agent]-[mGs]-[mCs]-[mAs]-[mGs]-[Ligand] |
| C-8 | [Nucleic Acid Agent]-[Ligand]-[[mGs]-[mCs]-[mAs]-[mGs]-[mCs]-[mCs]-[mGs]- |
| | [mAs]-[mAs]-[mAs]-[mCs]-[mCs]-[mGs]-[mAs]-[mCs] (SEQ ID NO: 1)] |
| C-9 | [Ligand]-[Nucleic Acid Agent]-[[mGs]-[mCs]-[mAs]-[mGs]-[mCs]-[mCs]-[mGs]- |
| | [mAs]-[mAs]-[mAs]-[mCs]-[mCs]-[mGs]-[mAs]-[mCs] (SEQ ID NO: 1)] |
| C- | [[mGs]-[mCs]-[mAs]-[mGs]-[mCs]-[mCs]-[mGs]-[mAs]-[mAs]-[mAs]-[mCs]- |
| 10 | [mCs]-[mGs]-[mAs]-[mCs] (SEQ ID NO: 1)]-[Nucleic Acid Agent]-[Ligand] |
| C- | [[mGs]-[mCs]-[mAs]-[mGs]-[mCs]-[mCs]-[mGs]-[mAs]-[mAs]-[mAs]-[mCs]- |
| 11 | [mCs]-[mGs]-[mAs]-[mCs] (SEQ ID NO: 1)]- [Ligand]-[Nucleic Acid Agent] |
| C- | [mGs]-[mCs]-[mAs]-[mGs]-[mCs]-[mCs]-[mGs]-[mAs]-[Ligand]-[mAs]-[mAs]- |
| 12 | [mCs]-[mCs]-[mGs]-[mAs]-[mC]-[Nucleic Acid Agent] |
| C- | [Ligand]-[mGs]-[mCs]-[mAs]-[mGs]-[mCs]-[mCs]-[mGs]-[mAs]-[mAs]-[Nucleic |
| 13 | Acid Agent] |
| C- | [Nucleic Acid Agent]-[Ligand]-[mGs]-[mCs]-[mAs]-[mGs]-[mCs]-[mCs]-[mGs]- |
| 14 | [mAs]-[mAs] |
| C- | [Ligand]-[Nucleic Acid Agent]-[mGs]-[mCs]-[mAs]-[mGs]-[mCs]-[mCs]-[mGs]- |
| 15 | [mAs]-[mAs] |
| C- | [mGs]-[mCs]-[mAs]-[mGs]-[mCs]-[mCs]-[mGs]-[mAs]-[mAs]-[Nucleic Acid |
| 16 | Agent]-[Ligand] |
| C- | [mGs]-[mCs]-[mAs]-[mGs]-[mCs]-[mCs]-[mGs]-[mAs]-[mAs]-[Ligand]-[Nucleic |
| 17 | Acid Agent] |
| C- | [Ligand]-[Abs]-[Abs]-[Abs]-[Abs]-[Abs]-[Abs]-[Abs]-[Abs]-[Abs]-[Nucleic Acid |
| 18 | Agent] |
| C- | [Nucleic Acid Agent]-[Ligand]-[Abs]-[Abs]-[Abs]-[Abs]-[Abs]-[Abs]-[Abs]- |
| 19 | [Abs]-[Abs] |
| C- | [Ligand]-[Nucleic Acid Agent]-[Abs]-[Abs]-[Abs]-[Abs]-[Abs]-[Abs]-[Abs]- |
| 20 | [Abs]-[Abs] |
| C- | [Abs]-[Abs]-[Abs]-[Abs]-[Abs]-[Abs]-[Abs]-[Abs]-[Abs]-[Ligand]-[Nucleic Acid |
| 21 | Agent] |
| C- | [Abs]-[Abs]-[Abs]-[Abs]-[Abs]-[Abs]-[Abs]-[Abs]-[Abs]-[Nucleic Acid Agent]- |
| 22 | [Ligand] |
| C- | [Ligand]-[mGs]-[mCs]-[mAs]-[mGs]-[mCs]-[mCs]-[Nucleic Acid Agent] |
| 23 | |
| C- | [Nucleic Acid Agent]-[Ligand]-[mGs]-[mCs]-[mAs]-[mGs]-[mCs]-[mCs] |
| 24 | |
| C- | [Ligand]-[Nucleic Acid Agent]-[mGs]-[mCs]-[mAs]-[mGs]-[mCs]-[mCs] |
| 25 | |
| C- | [mGs]-[mCs]-[mAs]-[mGs]-[mCs]-[mCs]-[Nucleic Acid Agent]-[Ligand] |
| 26 | |
| C- | [mGs]-[mCs]-[mAs]-[mGs]-[mCs]-[mCs]-[Ligand]-[Nucleic Acid Agent] |
| 27 | |
| C- | [Ligand]-[mGs]-[mCs]-[mAs]-[mGs]-[Nucleic Acid Agent] |
| 28 | |
| C- | [Ligand]-[Nucleic Acid Agent]-[mGs]-[mCs]-[mAs]-[mGs] |
| 29 | |
| C- | [Nucleic Acid Agent]-[Ligand]-[mGs]-[mCs]-[mAs]-[mGs] |
| 30 | |
| C- | [mGs]-[mCs]-[mAs]-[mGs]-[Nucleic Acid Agent]-[Ligand] |
| 31 | |
| C- | [mGs]-[mCs]-[mAs]-[mGs]-[Ligand]-[Nucleic Acid Agent] |
| 32 | |

In some embodiments, the conjugate is selected from the structures described in Table C, wherein [Nucleic Acid Agent] is the antisense strand of the Nucleic Acid Agent.

In some embodiments, the conjugate is selected from the structures described in Table C, wherein [Nucleic Acid Agent] is the sense strand of the Nucleic Acid Agent.

### Nucleotide-Based Enhancement Units

As used herein, a “Nucleotide-Based Enhancement Unit” refers to a moiety corresponding to a Nucleotide-Based Enhancement Agent in which the * in the nucleotide at the 5′-end and/or the ** in the nucleotide at the 2′- or 3′-end are/is attached to a Ligand, a Linker Unit, and/or a Nucleic Acid Agent.

In some embodiments, the Nucleotide-Based Enhancement Unit corresponds to a Nucleotide-Based Enhancement Agent in which the * in the nucleotide at the 5′-end is attached to a Ligand, a Linker Unit, and/or a Nucleic Acid Agent.

In some embodiments, the Nucleotide-Based Enhancement Unit corresponds to a Nucleotide-Based Enhancement Agent in which the ** in the nucleotide at the 2′- or 3′-end is attached to a Ligand, a Linker Unit, and/or a Nucleic Acid Agent.

In some embodiments, the Nucleotide-Based Enhancement Unit corresponds to a Nucleotide-Based Enhancement Agent in which the * in the nucleotide at the 5′-end is attached to a Ligand, a Linker Unit, and/or a Nucleic Acid Agent, and the ** in the nucleotide at the 2′- or 3′-end is attached to a Ligand, a Linker Unit, and/or a Nucleic Acid Agent.

In some embodiments, at least one nucleotide in the Nucleotide-Based Enhancement Unit is of Formula (I) or (II), wherein:
the * is attached to the 2′- or 3′-position of another nucleotide of the Nucleotide-Based Enhancement Unit, or is H. or is attached to a Ligand, a Linker Unit, and/or a Nucleic Acid Agent when the nucleotide is at the 5′-end of the Nucleotide-Based Enhancement Unit; and
the ** is attached to the 5′-position of another nucleotide of the Nucleotide-Based Enhancement Unit, or is H, or is attached to a Ligand, a Linker Unit, and/or a Nucleic Acid Agent when the nucleotide is at the 2- or 3′-end of the Nucleotide-Based Enhancement Unit.

In some embodiments, at least one nucleotide in the Nucleotide-Based Enhancement Unit is of Formula (I′) or (II′), wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, at least one nucleotide in the Nucleotide-Based Enhancement Unit is of Formula (I-A), (II-A), (III-A), or (TV-A), wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, at least one nucleotide in the Nucleotide-Based Enhancement Unit is of Formula (I′-A), (II′-A), (III′-A), or (IV′-A), wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, at least one nucleotide in the Nucleotide-Based Enhancement Unit is of Formula (I-B) or (I-B), wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, at least one nucleotide in the Nucleotide-Based Enhancement Unit is of Formula (I′-B) or (II′-B), wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, at least one nucleotide in the Nucleotide-Based Enhancement Unit is of Formula (I-C), (II-C), (III-C), or (IV-C), wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, at least one nucleotide in the Nucleotide-Based Enhancement Unit is of Formula (I′-C), (II′-C), (III′-C), or (IV′-C), wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, at least one nucleotide in the Nucleotide-Based Enhancement Unit is selected from Table L, wherein:
the * is attached to the 2′- or 3′-position of another nucleotide of the Nucleotide-Based Enhancement Unit, or is H or attached to a Ligand, a Linker Unit, and/or a Nucleic Acid when the nucleotide is at the 5′-end of the Nucleotide-Based Enhancement Unit; and
the ** is attached to the 5′-position of another nucleotide of the Nucleotide-Based Enhancement Unit, or is H or attached to a Ligand, a Linker Unit, and/or a Nucleic Acid when the nucleotide is at the 2′- or 3′-end of the Nucleotide-Based Enhancement Unit.

In some embodiments, the Nucleotide-Based Enhancement Unit comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides.

Tn some embodiments, the Nucleotide-Based Enhancement Unit comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides.

In some embodiments, the Nucleotide-Based Enhancement Unit comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides.

In some embodiments, the Nucleotide-Based Enhancement Unit comprises 5 nucleotides. In some embodiments, the Nucleotide-Based Enhancement Unit comprises 6 nucleotides. In some embodiments, the Nucleotide-Based Enhancement Unit comprises 7 nucleotides. In some embodiments, the Nucleotide-Based Enhancement Unit comprises 8 nucleotides. In some embodiments, the Nucleotide-Based Enhancement Unit comprises 9 nucleotides. In some embodiments, the Nucleotide-Based Enhancement Unit comprises 10 nucleotides. In some embodiments, the Nucleotide-Based Enhancement Unit comprises 11 nucleotides. In some embodiments, the Nucleotide-Based Enhancement Unit comprises 12 nucleotides. In some embodiments, the Nucleotide-Based Enhancement Unit comprises 13 nucleotides.

In some embodiments, the Nucleotide-Based Enhancement Unit comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides.

In some embodiments, the Nucleotide-Based Enhancement Unit comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Unit comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides.

In some embodiments, the Nucleotide-Based Enhancement Unit comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides, wherein the Nucleotide-Based Enhancement Unit further comprises one or more phosphodiester linkers and/or one or more phosphorothioate linkers.

In some embodiments, the Nucleotide-Based Enhancement Unit comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides, wherein the Nucleotide-Based Enhancement Unit further comprises one or more phosphodiester linkers and/or one or more phosphorothioate linkers.

In some embodiments, the Nucleotide-Based Enhancement Unit comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides, wherein the Nucleotide-Based Enhancement Unit further comprises one or more phosphodiester linkers and/or one or more phosphorothioate linkers.

In some embodiments, the Nucleotide-Based Enhancement Unit comprises 5 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 6 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 7 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 8 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 9 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 10 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 11 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 12 nucleotides, wherein the Nucleotide-Based Enhancement Unit further comprises one or more phosphodiester linkers and/or one or more phosphorothioate linkers. In some embodiments, the Nucleotide-Based Enhancement Unit comprises 13 nucleotides, wherein the Nucleotide-Based Enhancement Unit further comprises one or more phosphodiester linkers and/or one or more phosphorothioate linkers.

In some embodiments, the Nucleotide-Based Enhancement Unit comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides, wherein the Nucleotide-Based Enhancement Unit further comprises one or more phosphodiester linkers and/or one or more phosphorothioate linkers.

In some embodiments, the Nucleotide-Based Enhancement Unit comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Unit comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides, wherein the Nucleotide-Based Enhancement Unit further comprises one or more phosphodiester linkers and/or one or more phosphorothioate linkers.

Tn some embodiments, the Nucleotide-Based Enhancement Unit comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides, wherein the Nucleotide-Based Enhancement Unit further comprises one or more phosphodiester linkers.

In some embodiments, the Nucleotide-Based Enhancement Unit comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides, wherein the Nucleotide-Based Enhancement Unit further comprises one or more phosphodiester linkers.

In some embodiments, the Nucleotide-Based Enhancement Unit comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides, wherein the Nucleotide-Based Enhancement Unit further comprises one or more phosphodiester linkers.

In some embodiments, the Nucleotide-Based Enhancement Unit comprises 5 nucleotides, wherein the Nucleotide-Based Enhancement Unit further comprises one or more phosphodiester linkers. In some embodiments, the Nucleotide-Based Enhancement Unit comprises 6 nucleotides, wherein the Nucleotide-Based Enhancement Unit further comprises one or more phosphodiester linkers. In some embodiments, the Nucleotide-Based Enhancement Unit comprises 7 nucleotides, wherein the Nucleotide-Based Enhancement Unit further comprises one or more phosphodiester linkers. In some embodiments, the Nucleotide-Based Enhancement Unit comprises 8 nucleotides, wherein the Nucleotide-Based Enhancement Unit further comprises one or more phosphodiester linkers. In some embodiments, the Nucleotide-Based Enhancement Unit comprises 9 nucleotides, wherein the Nucleotide-Based Enhancement Unit further comprises one or more phosphodiester linkers. In some embodiments, the Nucleotide-Based Enhancement Unit comprises 10 nucleotides, wherein the Nucleotide-Based Enhancement Unit further comprises one or more phosphodiester linkers. In some embodiments, the Nucleotide-Based Enhancement Unit comprises 11 nucleotides, wherein the Nucleotide-Based Enhancement Unit further comprises one or more phosphodiester linkers. In some embodiments, the Nucleotide-Based Enhancement Unit comprises 12 nucleotides, wherein the Nucleotide-Based Enhancement Unit further comprises one or more phosphodiester linkers. In some embodiments, the Nucleotide-Based Enhancement Unit comprises 13 nucleotides, wherein the Nucleotide-Based Enhancement Unit further comprises one or more phosphodiester linkers.

In some embodiments, the Nucleotide-Based Enhancement Unit comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides, wherein the Nucleotide-Based Enhancement Unit further comprises one or more phosphodiester linkers.

In some embodiments, the Nucleotide-Based Enhancement Unit comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Unit comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides, wherein the Nucleotide-Based Enhancement Unit further comprises one or more phosphodiester linkers.

In some embodiments, the Nucleotide-Based Enhancement Unit comprises from 2 to 28 nucleotides, from 2 to 26 nucleotides, from 2 to 24 nucleotides, from 2 to 22 nucleotides, from 2 to 20 nucleotides, from 2 to 18 nucleotides, from 2 to 16 nucleotides, from 2 to 14 nucleotides, from 2 to 12 nucleotides, from 2 to 10 nucleotides, from 2 to 9 nucleotides, from 2 to 8 nucleotides, from 2 to 7 nucleotides, from 2 to 6 nucleotides, from 2 to 5 nucleotides, from 2 to 4 nucleotides, or from 2 to 3 nucleotides, wherein the Nucleotide-Based Enhancement Unit further comprises one or more phosphorothioate linkers.

In some embodiments, the Nucleotide-Based Enhancement Unit comprises from 4 to 30 nucleotides, from 6 to 30 nucleotides, from 8 to 30 nucleotides, from 10 to 30 nucleotides, from 12 to 30 nucleotides, from 14 to 30 nucleotides, from 16 to 30 nucleotides, from 18 to 30 nucleotides, from 20 to 30 nucleotides, from 21 to 30 nucleotides, from 22 to 30 nucleotides, from 23 to 30 nucleotides, from 24 to 30 nucleotides, from 25 to 30 nucleotides, from 26 to 30 nucleotides, from 27 to 30 nucleotides, from 28 to 30 nucleotides, or from 29 to 30 nucleotides, wherein the Nucleotide-Based Enhancement Unit further comprises one or more phosphorothioate linkers.

In some embodiments, the Nucleotide-Based Enhancement Unit comprises 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides, wherein the Nucleotide-Based Enhancement Unit further comprises one or more phosphorothioate linkers.

In some embodiments, the Nucleotide-Based Enhancement Unit comprises 5 nucleotides, wherein the Nucleotide-Based Enhancement Unit further comprises one or more phosphorothioate linkers. In some embodiments, the Nucleotide-Based Enhancement Unit comprises 6 nucleotides, wherein the Nucleotide-Based Enhancement Unit further comprises one or more phosphorothioate linkers. In some embodiments, the Nucleotide-Based Enhancement Unit comprises 7 nucleotides, wherein the Nucleotide-Based Enhancement Unit further comprises one or more phosphorothioate linkers. In some embodiments, the Nucleotide-Based Enhancement Unit comprises 8 nucleotides, wherein the Nucleotide-Based Enhancement Unit further comprises one or more phosphorothioate linkers. In some embodiments, the Nucleotide-Based Enhancement Unit comprises 9 nucleotides, wherein the Nucleotide-Based Enhancement Unit further comprises one or more phosphorothioate linkers. In some embodiments, the Nucleotide-Based Enhancement Unit comprises 10 nucleotides, wherein the Nucleotide-Based Enhancement Unit further comprises one or more phosphorothioate linkers. In some embodiments, the Nucleotide-Based Enhancement Unit comprises 11 nucleotides, wherein the Nucleotide-Based Enhancement Unit further comprises one or more phosphorothioate linkers. In some embodiments, the Nucleotide-Based Enhancement Unit comprises 12 nucleotides, wherein the Nucleotide-Based Enhancement Unit further comprises one or more phosphorothioate linkers. In some embodiments, the Nucleotide-Based Enhancement Unit comprises 13 nucleotides, wherein the Nucleotide-Based Enhancement Unit further comprises one or more phosphorothioate linkers.

In some embodiments, the Nucleotide-Based Enhancement Unit comprises from 2 to 21 nucleotides, 3 to 20 nucleotides, 4 to 19 nucleotides, 5 to 18 nucleotides, 6 to 17 nucleotides, 7 to 16 nucleotides, 8 to 15 nucleotides, 9 to 14 nucleotides, 10 to 13 nucleotides, or 11 to 12 nucleotides, wherein the Nucleotide-Based Enhancement Unit further comprises one or more phosphorothioate linkers.

In some embodiments, the Nucleotide-Based Enhancement Unit comprises from 2 to 19 nucleotides, 3 to 18 nucleotides, 4 to 17 nucleotides, 5 to 16 nucleotides, 6 to 15 nucleotides, 7 to 14 nucleotides, 8 to 13 nucleotides, 9 to 12 nucleotides, or 10 to 11 nucleotides. In some embodiments, the Nucleotide-Based Enhancement Unit comprises from 2 to 16 nucleotides, 3 to 15 nucleotides, 4 to 14 nucleotides, 5 to 13 nucleotides, 6 to 12 nucleotides, 7 to 11 nucleotides, 8 to 10 nucleotides, or 9 to 11 nucleotides, wherein the Nucleotide-Based Enhancement Unit further comprises one or more phosphorothioate linkers.

### Linker Units

As used herein, a “Linker Unit” or “linker unit” refers to a moiety with an attachment to a Nucleotide-Based Enhancement Unit, a Ligand, and/or a Nucleic Acid Agent.

In some embodiments, the Linker Unit has an attachment to a Nucleotide-Based Enhancement Unit and a Ligand.

In some embodiments, the Linker Unit has an attachment to a Ligand and a Nucleic Acid Agent.

In some embodiments, the Linker Unit has an attachment to a Nucleotide-Based Enhancement Unit and a Nucleic Acid Agent.

In some embodiments, the Linker Unit is a ribose derivative.

In some embodiments, the Linker Unit is a 1′-alkyl modified ribose derivative, e.g., as described in PCT Application No. PCT/US2022/039517 (incorporated herein by reference).

In some embodiments, the Linker Unit is a 2′-alkyl or 3′-alkyl modified ribose derivative, e.g., as described in PCT Application No. PCT/US2022/044377 (incorporated herein by reference).

In some embodiments, the Linker Unit is a polyhydroxylated cyclopentane derivative, e.g., as described in PCT Application No. PCT/US2022/045748 (incorporated herein by reference).

### Ligands

As used herein, the term “Ligand” or “ligand” refers to a moiety that, when being covalently attached to a Nucleic Acid Agent (e.g., an oligonucleotide), is capable of mediating its entry into, or facilitating its delivery to, a target site (e.g., a target cell or tissue).

In some embodiments, the ligand comprises a sugar ligand moiety (e.g., N-acetylgalactosamine (GalNAc)) which may direct uptake of an oligonucleotide into the liver.

In some embodiments, the ligand binds to the asialoglycoprotein receptor (ASGPR). In some embodiments, the ligand binds to (e.g., through ASGPR) the liver, such as the parenchymal cells of the liver.

In some embodiments, the ligand is capable of binding to a glucagon-like peptide receptor, e.g., glucagon-like peptide-1 receptor (GLP-1 receptor). In some embodiments, the ligand binds to a glucagon-like peptide-1 receptor (GLP-1 receptor). In some embodiments, the ligand binds to (e.g., through the GLP-1 receptor) the pancreas, such as the beta cells of the pancreas.

In some embodiments, the ligand is a small molecule, carbohydrate, oligonucleotide, antibody, or peptide.

In some embodiments, the ligand is a peptide capable of binding to a glucagon-like peptide receptor, e.g., glucagon-like peptide-1 receptor (GLP-1 receptor).

Suitable ligands include, but are not limited to, the ligands disclosed in Winkler (Ther. Deliv., 2013, 4(7): 791-809); Mayendraraj et al. (Peptides, 2022, 170749); Willard et al. (Exp. Diabetes Res., 2012; 2012; 709893); Sloop et al. (Diabetes, 2010, 59(12): 3099-3107); Knudsen et al. (PNAS, 2007, 104(3): 937-942); and Wang et al. (Acta Pharmacol. Sin. 2010, 31: 1026-1030), PCT Patent Appl'n Pub. Nos. WO/2005/018536, WO/2008/086086, WO/2011/075393, WO/2011/056644, WO/2016/100401, WO/2012/089352, WO/2009/082607, and WO/2019/092618, U.S. Patent Appl'n Pub. Nos. 2006/0275288, 2007/124461, 2009/0239814, 2012/0136042, 2013/0158824, 2014/0206607, and 2009/0247608, and U.S. Pat. Nos. 9,187,522, 8,329,419, and 8,389,689, each of which is incorporated by reference.

In some embodiments, the ligand comprises a carbohydrate moiety.

As used herein, “carbohydrate moiety” refers to a moiety which comprises one or more monosaccharide units each having at least six carbon atoms (which may be linear, branched or cyclic), with an oxygen, nitrogen or sulfur atom bonded to each carbon atom. In some embodiments, the carbohydrate moiety comprises a monosaccharide, a disaccharide, a trisaccharide, or a tetrasaccharide. In some embodiments, the carbohydrate moiety comprises an oligosaccharide containing from about 4-9 monosaccharide units. In some embodiments, the carbohydrate moiety comprises a polysaccharide (e.g., a starch, a glycogen, a cellulose, or a polysaccharide gum).

In some embodiments, the carbohydrate moiety comprises a monosaccharide, a disaccharide, a trisaccharide, or a tetrasaccharide.

In some embodiments, the carbohydrate moiety comprises an oligosaccharide (e.g., containing from about four to about nine monosaccharide units).

In some embodiments, the carbohydrate moiety comprises a polysaccharide (e.g., a starch, a glycogen, a cellulose, or a polysaccharide gum).

In some embodiments, the ligand is capable of binding to a human asialoglycoprotein receptor (ASGPR), e.g., human asialoglycoprotein receptor 2 (ASGPR2).

In some embodiments, the carbohydrate moiety comprises a sugar (e.g., one, two, or three sugar).

In some embodiments, the carbohydrate moiety comprises galactose or a derivative thereof (e.g., one, two, or three galactose or the derivative thereof).

In some embodiments, the carbohydrate moiety comprises N-acetylgalactosamine or a derivative thereof (e.g., one, two, or three N-acetylgalactosamine or the derivative thereof).

In some embodiments, the carbohydrate moiety comprises N-acetyl-D-galactosylamine or a derivative thereof (e.g., one, two, or three N-acetyl-D-galactosylamine or the derivative thereof).

In some embodiments, the carbohydrate moiety comprises N-acetylgalactosamine (e.g., one, two, or three N-acetylgalactosamine).

In some embodiments, the carbohydrate moiety comprises N-acetyl-D-galactosylamine (e.g., one, two, or three N-acetyl-D-galactosylamine).

In some embodiments, the carbohydrate moiety comprises mannose or a derivative thereof (e.g., mannose-6-phosphate).

In some embodiments, the carbohydrate moiety further comprises a linking moiety that connects the one or more sugar (e.g., N-acetyl-D-galactosylamine) with the Linker Unit.

In some embodiments the linking moiety comprises thioether (e.g., thiosuccinimide, or the hydrolysis analogue thereof), disulfide, triazole, phosphorothioate, phosphodiester, ester, amide, or any combination thereof.

In some embodiments, the linking moiety is a triantennary linking moiety.

Suitable ligands include, but are not limited to, the ligands disclosed in PCT Appl'n Pub. Nos. WO/2015/006740, WO/2016/100401, WO/2017/214112, WO/2018/039364, and WO/2018/045317, each of which is incorporated herein by reference.

In some embodiments, the ligand comprises

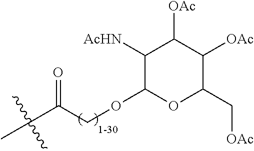

(e.g., one, two, or three

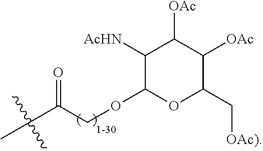

In some embodiments, the ligand comprises

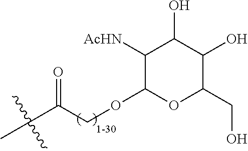

(e.g., one, two, or three

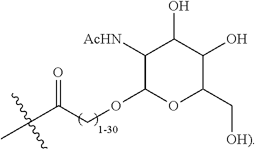

In some embodiments, the ligand comprises

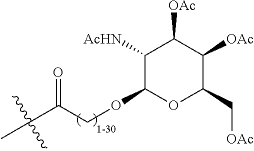

(e.g., one, two, or three

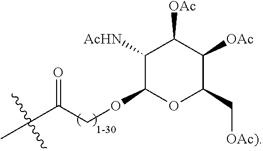

In some embodiments, the ligand comprises

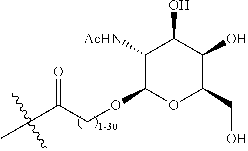

(e.g., one, two, or three

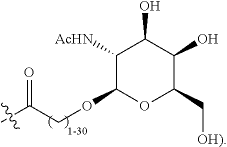

In some embodiments, the ligand comprises

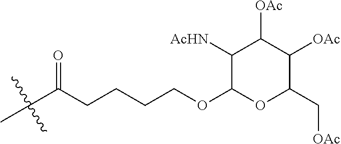 [SMILES: *C(=O)CCCCOC1OC(COC(C)=O)C(OC(C)=O)C(OC(C)=O)C1NC(C)=O]

(e.g., one, two, or three

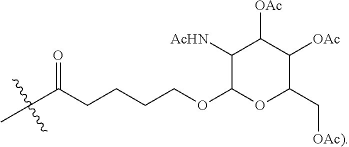

In some embodiments, the ligand comprises

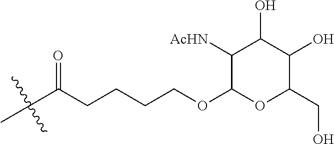 [SMILES: *C(=O)CCCCOC1OC(CO)C(O)C(O)C1NC(C)=O]

(e.g., one, two, or three

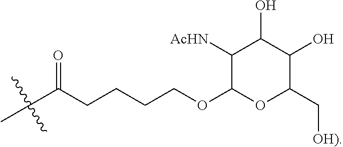

In some embodiments, the ligand comprises

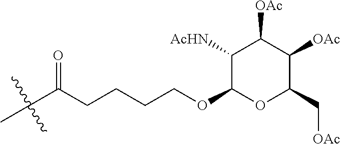 [SMILES: *C(=O)CCCCO[C@@H]1O[C@H](COC(C)=O)[C@H](OC(C)=O)[C@H](OC(C)=O)[C@H]1NC(C)=O]

(e.g., one, two, or three

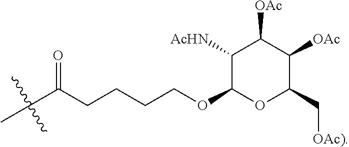

In some embodiments, the ligand comprises

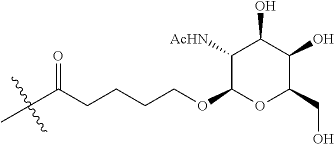 [SMILES: *C(=O)CCCCO[C@@H]1O[C@H](CO)[C@H](O)[C@H](O)[C@H]1NC(C)=O]

(e.g., one, two, or three

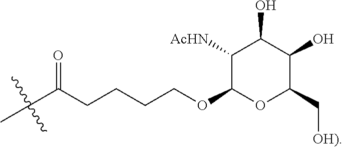

In some embodiments, the ligand comprises

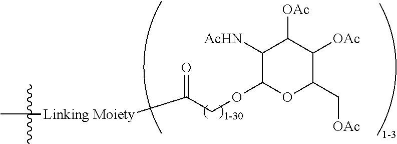

In some embodiments, the ligand comprises

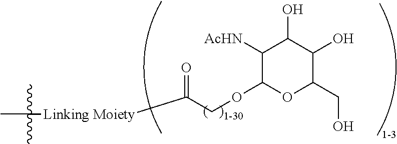

In some embodiments, the ligand comprises

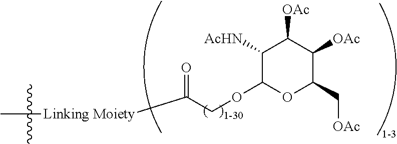

In some embodiments, the ligand comprises

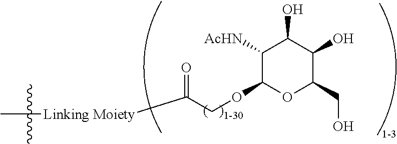

In some embodiments, the ligand comprises

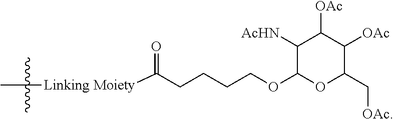

In some embodiments, the ligand comprises

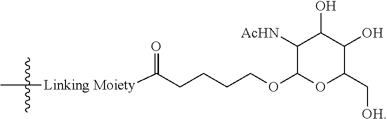

In some embodiments, the ligand comprises

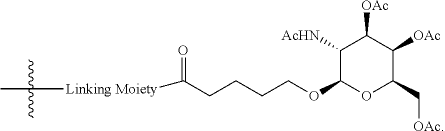

In some embodiments, the ligand comprises

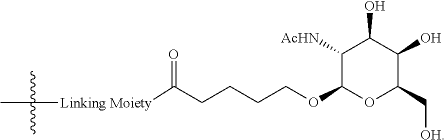

In some embodiments, the ligand comprises a lipid moiety (e.g., one, two, or three lipid moiety).

In some embodiments the lipid moiety comprises (e.g., one, two, of three of) C₈-C₂₄ fatty acid, cholesterol, vitamin, sterol, phospholipid, or any combination thereof.

In some embodiments, the ligand comprises a peptide moiety (e.g., one, two, or three peptide moiety). In some embodiments, the ligand comprises a peptide moiety (e.g., one, two, or three peptide moiety) capable of binding to a glucagon-like peptide receptor, e.g., glucagon-like peptide-1 receptor (GLP-1 receptor).

In some embodiments, the peptide moiety comprises (e.g., one, two, or three of) integrin, insulin, glucagon-like peptide (e.g., GLP-1), or any combination thereof.

In some embodiments, the peptide moiety comprises (e.g., one, two, or three of) glucagon-like peptide (e.g., GLP-1). In some embodiments, the peptide moiety comprises (e.g., one, two, or three of) glucagon.

In some embodiments, the peptide moiety comprises (e.g., one, two, or three of) dulaglutide. In some embodiments, the peptide moiety comprises (e.g., one, two, or three of) lixisenatide. In some embodiments, the peptide moiety comprises (e.g., one, two, or three of) exenatide. In some embodiments, the peptide moiety comprises (e.g., one, two, or three of) semaglutide. In some embodiments, the peptide moiety comprises (e.g., one, two, or three of) albiglutide. In some embodiments, the peptide moiety comprises (e.g., one, two, or three of) liraglutide. In some embodiments, the peptide moiety comprises (e.g., one, two, or three of) tirzepatide. In some embodiments, the peptide moiety comprises (e.g., one, two, or three of) avexitide. In some embodiments, the peptide moiety comprises (e.g., one, two, or three of) HISHS-2001. In some embodiments, the peptide moiety comprises (e.g., one, two, or three of) MAR709. In some embodiments, the peptide moiety comprises (e.g., one, two, or three of) exendin-4.

In some embodiments, the ligand comprises an antibody moiety (e.g., transferrin). In some embodiments, the ligand comprises an antibody moiety capable of binding to a glucagon-like peptide receptor, e.g., glucagon-like peptide-1 receptor (GLP-1 receptor).

In some embodiments, the ligand comprises one, two, or three antibody moiety (e.g., transferrin). In some embodiments, the ligand comprises one, two, or three antibody moieties capable of binding to a glucagon-like peptide receptor, e.g., glucagon-like peptide-1 receptor (GLP-1 receptor).

In some embodiments, the ligand comprises an oligonucleotide (e.g., aptamer or CpG). In some embodiments, the ligand comprises an oligonucleotide (e.g., aptamer or CpG) capable of binding to a glucagon-like peptide receptor, e.g., glucagon-like peptide-1 receptor (GLP-1 receptor).

In some embodiments, the ligand comprises one, two, or three oligonucleotide (e.g., aptamer or CpG). In some embodiments, the ligand comprises one, two, or three oligonucleotide (e.g., aptamer or CpG) capable of binding to a glucagon-like peptide receptor, e.g., glucagon-like peptide-1 receptor (GLP-1 receptor).

In some embodiments, the ligand comprises a small molecule moiety. In some embodiments, the ligand comprises a small molecule moiety capable of binding to a glucagon-like peptide receptor, e.g., glucagon-like peptide-1 receptor (GLP-1 receptor).

In some embodiments, the ligand comprises one, two, or three small molecule moiety. In some embodiments, the ligand comprises one, two, or three small molecule moiety capable of binding to a glucagon-like peptide receptor, e.g., glucagon-like peptide-1 receptor (GLP-1 receptor).

In some embodiments, the ligand comprises:
one, two, or three sugar (e.g., N-acetyl-D-galactosylamine);
one, two, or three lipid moiety;
one, two, or three peptide moiety;
one, two, or three antibody moiety;
one, two, or three oligonucleotide; or
any combination thereof.

In some embodiments, the ligand comprises:
one, two, or three carbohydrate moiety;
one, two, or three peptide moiety;
one, two, or three antibody moiety;
one, two, or three small molecule moiety; or
any combination thereof.

### Nucleic Acid Agents

In some embodiments, the Nucleic Acid Agent comprises an oligonucleotide.

In some embodiments, the Nucleic Acid Agent (e.g., the oligonucleotide) comprises one or more one or more phosphate groups or one or more analogs of a phosphate group.

In some embodiments, the Linker Unit is attached to the Nucleic Acid Agent (e.g., the oligonucleotide) via a phosphate group, or an analog of a phosphate group, in the Nucleic Acid Agent.

In some embodiments, the oligonucleotide has a length of from 1 to 40 nucleotides, from 10 to 40 nucleotides, from 12 to 35 nucleotides, from 15 to 30 nucleotides, from 18 to 25 nucleotides, or from 20 to 23 nucleotides. In some embodiments, the oligonucleotide has a length of 18, 19, 20, 21, 22, 23, 24, or 25 nucleotides. In some embodiments, the oligonucleotide has a length of 20, 21, 22, or 23 nucleotides.

In some embodiments, the Nucleic Acid Agent comprises an RNA, a DNA, or a mixture thereof.

In some embodiments, the Nucleic Acid Agent comprises an RNA.

In some embodiments, the oligonucleotide is an siRNA (e.g., a single strand siRNA (e.g., a hairpin single strand siRNA) or a double strand siRNA), microRNA, antimicroRNA, microRNA mimic, antagomir, dsRNA, ssRNA, aptamer, immune stimulatory oligonucleotide, decoy oligonucleotide, splice altering oligonucleotide, triplex forming oligonucleotide, G-quadruplexe, or antisense oligonucleotide.

In some embodiments, the Nucleic Acid Agent comprises a double stranded RNA (dsRNA), wherein the double stranded RNA comprises a sense strand and an antisense strand, as described herein.

In some embodiments, the Nucleic Acid Agent comprises a double stranded siRNA (ds-siRNA), wherein the double stranded siRNA comprises a sense strand and an antisense strand, as described herein.

It is understood that sense strand is also known as passenger strand, and the terms “sense strand” and “passenger strand” are used interchangeably herein.

It is understood that antisense strand is also known as guide strand, and the terms “antisense strand” and “guide strand” are used interchangeably herein.

In some embodiments, the oligonucleotide is an iRNA.

The term “iRNA” refers to an RNA agent which can down regulate the expression of a target gene (e.g., an siRNA), e.g., an endogenous or pathogen target RNA. While not wishing to be bound by theory, an iRNA may act by one or more of a number of mechanisms, including post-transcriptional cleavage of a target mRNA (referred to in the art as RNAi), or pre-transcriptional or pre-translational mechanisms. An iRNA can include a single strand or can include more than one strands, e.g., it can be a double stranded iRNA. If the iRNA is a single strand it can include a 5′ modification which includes one or more phosphate groups or one or more analogs of a phosphate group. In some embodiments, the iRNA is double stranded. In some embodiments, one or both strands of the double stranded iRNA can be modified, e.g., 5′ modification.

The iRNA typically includes a region of sufficient homology to the target gene, and is of sufficient length in terms of nucleotides, such that the iRNA, or a fragment thereof, can mediate down regulation of the target gene. The iRNA is or includes a region which is at least partially, and in some embodiments fully, complementary to the target RNA. It is not necessary that there be perfect complementarity between the iRNA and the target, but the correspondence may be sufficient to enable the iRNA, or a cleavage product thereof, to direct sequence specific silencing, e.g., by RNAi cleavage of the target RNA, e.g., mRNA.

The nucleotides in the iRNA may be modified (e.g., one or more nucleotides may include a 2′-F or 2′-OCH₃ group, or be nucleotide surrogates). The single stranded or double stranded regions of an iRNA may be modified or include nucleotide surrogates, e.g., the unpaired region or regions of a hairpin structure, e.g., a region which links two complementary regions, can have modifications or nucleotide surrogates. Modification to stabilize one or more 3′- or 5′-terminus of an iRNA, e.g., against exonucleases. Modifications can include C3 (or C6, C7, C12) amino linkers, thiol linkers, carboxyl linkers, non-nucleotide spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis. Modifications can also include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. In some embodiments, the different strands will include different modifications.

In some embodiments, the strands are chosen such that the iRNA includes a single strand or unpaired region at one or both ends of the molecule. A double stranded iRNA may have an overhang, e.g., one or two 5′ or 3′ overhangs (e.g., at least a 3′ overhang of 2-3 nucleotides). In some embodiments, the iRNA has overhangs, e.g., 3′ overhangs, of 1, 2, or 3 nucleotides in length at each end. The overhangs can be the result of one strand being longer than the other, or the result of two strands of the same length being staggered.

In some embodiments, the length for the duplexed regions between the strands of the iRNA are between 6 and 30 nucleotides in length. In some embodiments, the duplexed regions are between 15 and 30, most preferably 18, 19, 20, 21, 22, and 23 nucleotides in length. In some embodiments, the duplexed regions are between 6 and 20 nucleotides, most preferably 6, 7, 8, 9, 10, 11 and 12 nucleotides in length.

The oligonucleotide may be that described in U.S. Patent Publication Nos. 2009/0239814, 2012/0136042, 2013/0158824, or 2009/0247608, each of which is hereby incorporated by reference.

In some embodiments, the oligonucleotide is an siRNA.

In some embodiments, the oligonucleotide is a single strand siRNA.

In some embodiments, the oligonucleotide is a double strand siRNA, for example, double strand siRNA described herein.

A “single strand siRNA” as used herein, is an siRNA which is made up of a single strand, which includes a duplexed region, formed by intra-strand pairing, e.g., it may be, or include, a hairpin or pan-handle structure. Single strand siRNAs may be antisense with regard to the target molecule.

A single strand siRNA may be sufficiently long that it can enter the RISC and participate in RISC mediated cleavage of a target mRNA. A single strand siRNA is at least 14, and in some embodiments at least 15, 20, 25, 29, 35, 40, or 50 nucleotides in length. In some embodiments, it is less than 200, 100, 80, 60, 50, 40, or 30 nucleotides in length.

In some embodiments, the single strand siRNA has a length of from 10 to 40 nucleotides, from 12 to 35 nucleotides, from 15 to 30 nucleotides, from 18 to 25 nucleotides, or from 20 to 23 nucleotides. In some embodiments, the single strand siRNA has a length of 18, 19, 20, 21, 22, 23, 24, or 25 nucleotides. In some embodiments, the single strand siRNA has a length of 20, 21, 22, or 23 nucleotides.

Hairpin siRNAs may have a duplex region equal to or at least 17, 18, 19, 20, 21, 22, 23, 24, or 25 nucleotide pairs. The duplex region may be equal to or less than 200, 100, or 50 nucleotide pairs in length. In some embodiments, ranges for the duplex region are 15-30, 17 to 23, 19 to 23, and 19 to 21 nucleotides pairs in length. The hairpin may have a single strand overhang or terminal unpaired region. In some embodiments, the overhangs are 2-3 nucleotides in length. In some embodiments, the overhang is at the sense side of the hairpin and in some embodiments on the antisense side of the hairpin.

In some embodiments, the oligonucleotide is a double strand siRNA.

A “double stranded siRNA” as used herein, is an siRNA which includes more than one, and in some cases two, strands in which interchain hybridization can form a region of duplex structure.

In some embodiments, the sense strand of a double stranded siRNA may be equal to or at least 14, 15, 16 17, 18, 19, 20, 21, 22, 23, 24, 25, 29, 40, or 60 nucleotides in length. It may be equal to or less than 200, 100, or 50 nucleotides in length. Ranges may be 17 to 25, 19 to 23, 19 to 21, 21 to 23, or 20 to 22 nucleotides in length.

In some embodiments, the sense strand has a length of from 10 to 40 nucleotides, from 12 to 35 nucleotides, from 15 to 30 nucleotides, from 18 to 25 nucleotides, or from 20 to 23 nucleotides. In some embodiments, the sense strand has a length of 18, 19, 20, 21, 22, 23, 24, or 25 nucleotides. In some embodiments, the sense strand has a length of 20, 21, 22, or 23 nucleotides.

In some embodiments, the sense strand has a length of 18, 19, 20, 21, or 22 nucleotides.

In some embodiments, the antisense strand of a double stranded siRNA may be equal to or at least, 14, 15, 16 17, 18, 19, 20, 21, 22, 23, 24, 25, 29, 40, or 60 nucleotides in length. It may be equal to or less than 200, 100, or 50 nucleotides in length. Ranges may be 17 to 25, 19 to 23, 19 to 21, 21 to 23, or 20 to 22 nucleotides in length.

In some embodiments, the antisense strand has a length of from 10 to 40 nucleotides, from 12 to 35 nucleotides, from 15 to 30 nucleotides, from 18 to 25 nucleotides, or from 20 to 23 nucleotides. In some embodiments, the antisense strand has a length of 18, 19, 20, 21, 22, 23, 24, or 25 nucleotides. In some embodiments, the antisense strand has a length of 20, 21, 22, or 23 nucleotides.

In some embodiments, the antisense strand has a length of 20, 21, 22, 23, or 24 nucleotides.

In some embodiments, the sense strand has a length of 18, 19, 20, 21, or 22 nucleotides, and the antisense strand has a length of 20, 21, 22, 23, or 24 nucleotides.

In some embodiments, the sense strand has a length of 18 nucleotides, and the antisense strand has a length of 20 nucleotides.

In some embodiments, the sense strand has a length of 19 nucleotides, and the antisense strand has a length of 21 nucleotides.

In some embodiments, the sense strand has a length of 20 nucleotides, and the antisense strand has a length of 22 nucleotides.

In some embodiments, the sense strand has a length of 21 nucleotides, and the antisense strand has a length of 23 nucleotides.

In some embodiments, the sense strand has a length of 22 nucleotides, and the antisense strand has a length of 24 nucleotides.

The double strand portion of a double stranded siRNA may be equal to or at least, 14, 15, 16 17, 18, 19, 20, 21, 22, 23, 24, 25, 29, 40, or 60 nucleotide pairs in length. It may be equal to or less than 200, 100, or 50 nucleotides pairs in length. Ranges may be 15 to 30, 17 to 23, 19 to 23, and 19 to 21 nucleotides pairs in length.

In some embodiments, the siRNA is sufficiently large that it can be cleaved by an endogenous molecule, e.g., by Dicer, to produce smaller siRNAs, e.g., siRNAs agents.

The sense and antisense strands may be chosen such that the double-stranded siRNA includes a single strand or unpaired region at one or both ends of the molecule. Thus, a double-stranded siRNA may contain sense and antisense strands, paired to contain an overhang, e.g., one or two 5′ or 3′ overhangs, or a 3′ overhang of 1-3 nucleotides. The overhangs can be the result of one strand being longer than the other, or the result of two strands of the same length being staggered. Some embodiments will have at least one 3′ overhang. In some embodiments, both ends of an siRNA molecule will have a 3′ overhang. In some embodiments, the overhang is 2 nucleotides.

In some embodiments, the length for the duplexed region is between 15 and 30, or 18, 19, 20, 21, 22, and 23 nucleotides in length, e.g., in the ssiRNA range discussed above. ssiRNAs can resemble in length and structure the natural Dicer processed products from long dsiRNAs. Embodiments in which the two strands of the ssiRNA are attached, e.g., covalently attached are also included. Hairpin, or other single strand structures which provide the required double stranded region, and a 3′ overhang are also contemplated.

The siRNAs described herein, including double-stranded siRNAs and single-stranded siRNAs can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an ssiRNA of 21 to 23 nucleotides.

In some embodiments, an siRNA is “sufficiently complementary” to a target RNA, e.g., a target mRNA, such that the siRNA silences production of protein encoded by the target mRNA. In another embodiment, the siRNA is “exactly complementary” to a target RNA, e.g., the target RNA and the siRNA anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the siRNA specifically discriminates a single-nucleotide difference. In this case, the siRNA only mediates RNAi if exact complementary is found in the region (e.g., within 7 nucleotides of) the single-nucleotide difference.

MicroRNAs: Micro RNAs (miRNAs) are a highly conserved class of small RNA molecules that are transcribed from DNA in the genomes of plants and animals, but are not translated into protein. Processed miRNAs are single stranded ^{˜}17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S. NAR, 2004, 32, Database Issue, D109-D111.

Antisense Oligonucleosides: In some embodiments, a nucleic acid is an antisense oligonucleotide directed to a target polynucleotide. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence, e.g. a target gene mRNA. Antisense oligonucleotides are thought to inhibit gene expression by binding to a complementary mRNA. Binding to the target mRNA can lead to inhibition of gene expression either by preventing translation of complementary mRNA strands by binding to it, or by leading to degradation of the target mRNA. Antisense DNA can be used to target a specific, complementary (coding or non-coding) RNA. If binding takes places this DNA/RNA hybrid can be degraded by the enzyme RNase H. In some embodiments, antisense oligonucleotides contain from about 10 to about 50 nucleotides, more preferably about 15 to about 30 nucleotides. The term also encompasses antisense oligonucleotides that may not be exactly complementary to the desired target gene. Thus, instances where non-target specific-activities are found with antisense, or where an antisense sequence containing one or more mismatches with the target sequence is the most preferred for a particular use, are contemplated.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalacturonase and the muscarine type 2 acetylcholine receptor are inhibited by antisense oligonucleotides directed to their respective mRNA sequences (U.S. Pat. Nos. 5,739,119 and 5,759,829 each of which is incorporated by reference). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et al., Science. 1988 Jun. 10; 240(4858):1544-6; Vasanthakumar and Ahmed, Cancer Commun. 1989; 1(4):225-32; Peris et al., Brain Res Mol Brain Res. 1998 Jun. 15; 57(2):310-20; U.S. Pat. Nos. 5,801,154; 5,789,573; 5,718,709 and 5,610,288, each of which is incorporated by reference). Furthermore, antisense constructs have also been described that inhibit and can be used to treat a variety of abnormal cellular proliferations, e.g. cancer (U.S. Pat. Nos. 5,747,470; 5,591,317 and 5,783,683, each of which is incorporated by reference).

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

Antagomirs: Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety. Antagomir RNAs may be synthesized using standard solid phase oligonucleotide synthesis protocols. See U.S. Patent Application Publication Nos. 2007/0123482 and 2007/0213292 (each of which is incorporated herein by reference).

An antagomir can include ligand-conjugated monomer subunits and monomers for oligonucleotide synthesis. Exemplary monomers are described in U.S. Patent Application Publication No. 2005/0107325, which is incorporated by reference in its entirety. An antagomir can have a ZXY structure, such as is described in WO 2004/080406, which is incorporated by reference in its entirety. An antagomir can be complexed with an amphipathic moiety. Exemplary amphipathic moieties for use with oligonucleotide agents are described in WO 2004/080406, which is incorporated by reference in its entirety.

Aptamers: Aptamers are nucleic acid or peptide molecules that bind to a particular molecule of interest with high affinity and specificity (Tuerk and Gold, Science 249:505 (1990); Ellington and Szostak, Nature 346:818 (1990), each of which is incorporated by reference in its entirety). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9 (1999), and Hermann and Patel, Science 287:820-5 (2000), each of which is incorporated by reference in its entirety. Aptamers may be RNA or DNA based, and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, as described more fully herein, the term “aptamer” specifically includes “secondary aptamers” containing a consensus sequence derived from comparing two or more known aptamers to a given target.

Ribozymes: According to another embodiment, nucleic acid-lipid particles are associated with ribozymes. Ribozymes are RNA molecules complexes having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). For example, a large number of ribozymes accelerate phosphoester transfer reactions with a high degree of specificity, often cleaving only one of several phosphoesters in an oligonucleotide substrate (Cech et al., Cell. 1981 December; 27(3 Pt 2):487-96; Michel and Westhof, J Mol Biol. 1990 Dec. 5: 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis S virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis S virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in U.S. Pat. No. 4,987,071. Important characteristics of enzymatic nucleic acid molecules used are that they have a specific substrate binding site which is complementary to one or more of the target gene DNA or RNA regions, and that they have nucleotide sequences within or surrounding that substrate binding site which impart an RNA cleaving activity to the molecule. Thus the ribozyme constructs need not be limited to specific motifs mentioned herein.

Methods of producing a ribozyme targeted to any polynucleotide sequence are known in the art. Ribozymes may be designed as described in Int. Pat. Appl. Publ. Nos. WO 93/23569 and WO 94/02595, each specifically incorporated herein by reference, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. Nos. WO 92/07065, WO 93/15187, and WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Immunostimulatory Oligonucleotides: Nucleic acids associated with lipid particles may be immunostimulatory, including immunostimulatory oligonucleotides (ISS; single- or double-stranded) capable of inducing an immune response when administered to a subject, which may be a mammal or other patient. ISS include, e.g., certain palindromes leading to hairpin secondary structures (see Yamamoto S., et al. (1992) J. Immunol. 148: 4072-4076, which is incorporated by reference in its entirety), or CpG motifs, as well as other known ISS features (such as multi-G domains, see WO 96/11266, which is incorporated by reference in its entirety).

The immune response may be an innate or an adaptive immune response. The immune system is divided into a more innate immune system, and acquired adaptive immune system of vertebrates, the latter of which is further divided into humoral cellular components. In some embodiments, the immune response may be mucosal.

In some embodiments, an immunostimulatory nucleic acid is only immunostimulatory when administered in combination with a lipid particle, and is not immunostimulatory when administered in its “free form.” Such an oligonucleotide is considered to be immunostimulatory.

Immunostimulatory nucleic acids are considered to be non-sequence specific when it is not required that they specifically bind to and reduce the expression of a target polynucleotide in order to provoke an immune response. Thus, certain immunostimulatory nucleic acids may comprise a sequence corresponding to a region of a naturally occurring gene or mRNA, but they may still be considered non-sequence specific immunostimulatory nucleic acids.

In some embodiments, the immunostimulatory nucleic acid or oligonucleotide comprises at least one CpG dinucleotide. The oligonucleotide or CpG dinucleotide may be unmethylated or methylated. In another embodiment, the immunostimulatory nucleic acid comprises at least one CpG dinucleotide having a methylated cytosine. In some embodiments, the nucleic acid comprises a single CpG dinucleotide, wherein the cytosine in said CpG dinucleotide is methylated. In an alternative embodiment, the nucleic acid comprises at least two CpG dinucleotides, wherein at least one cytosine in the CpG dinucleotides is methylated. In a further embodiment, each cytosine in the CpG dinucleotides present in the sequence is methylated. In another embodiment, the nucleic acid comprises a plurality of CpG dinucleotides, wherein at least one of said CpG dinucleotides comprises a methylated cytosine.

### Attachments Between Nucleotide-Based Enhancement Unit, Linker Unit, Nucleic Acid Agent, and Ligand

In some embodiments, the attachment between the Nucleotide-Based Enhancement Unit and the Linker Unit is a bond.

In some embodiments, the attachment between the Nucleotide-Based Enhancement Unit and the Linker Unit is a moiety (e.g., a moiety comprising a cleavable group).

In some embodiments, the attachment between the Nucleotide-Based Enhancement Unit and the Linker Unit comprises —C(═O)— connected to the Linker Unit.

In some embodiments, the attachment between the Nucleotide-Based Enhancement Unit and the Nucleic Acid Agent is a bond.

In some embodiments, the attachment between the Nucleotide-Based Enhancement Unit and the Nucleic Acid Agent is a moiety (e.g., a moiety comprising a cleavable group).

In some embodiments, the attachment between the Nucleotide-Based Enhancement Unit and the Ligand is a bond.

In some embodiments, the attachment between the Nucleotide-Based Enhancement Unit and the Ligand is a moiety (e.g., a moiety comprising a cleavable group).

In some embodiments, the attachment between the Linker Unit and the Nucleic Acid Agent is a bond.

In some embodiments, the attachment between the Linker Unit and the Nucleic Acid Agent is a moiety (e.g., a moiety comprising a cleavable group).

In some embodiments, the attachment between the Linker Unit and the Ligand is a bond.

In some embodiments, the attachment between the Linker Unit and the Ligand is a moiety (e.g., a moiety comprising a cleavable group).

In some embodiments, the attachment between the Linker Unit and the Ligand comprises —C(═O)— connected to the Linker Unit.

The attachment between Nucleotide-Based Enhancement Unit, Linker Unit, Nucleic Acid Agent, and Ligand can be a cleavable group or a non-cleavable group. Suitable groups include, for example, —NR—, —C(═O)—, —C(═O)NH—, —S(═O)—, —S(═O)₂—, —S(═O)₂NH— or a chain of atoms, such as, but not limited to, alkylene, alkenylene alkynylene arylalkylene arylalkenylene arylalkynylene heteroarylalkylene heteroarylalkenylene heteroarylalkynylene heterocyclylalkylene heterocyclylalkenylene heterocyclylalkynylene arylene heteroarylene heterocyclylene cycloalkylene cycloalkenylene alkylarylalkylene alkylarylalkenylene alkylarylalkynylene alkenylarylalkylene alkenylarylalkenylene alkenylarylalkynylene alkynylarylalkylene alkynylarylalkenylene alkynylarylalkynylene alkylheteroarylalkylene alkylheteroarylalkenylene alkylheteroarylalkynylene alkenylheteroarylalkylene alkenylheteroarylalkenylene alkenylheteroarylalkynylene alkynylheteroarylalkylene alkynylheteroarylalkenylene alkynylheteroarylalkynylene alkylheterocyclylalkylene alkylheterocyclylalkenylene alkylhererocyclylalkynylene alkenylheterocyclylalkylene alkenylheterocyclylalkenylene alkenylheterocyclylalkynylene alkynylheterocyclylalkylene alkynylheterocyclylalkenylene alkynylheterocyclylalkynylene alkylarylene alkenylarylene alkynylarylene alkylheteroarylene alkenylheteroarylene alkynylhereroarylene each of which may be substituted or unsubstituted, and which one or more methylenes can be interrupted or terminated by —O—, —S—, —S(═O)—, —S(═O)₂—, —NR—, —C(═O)—, substituted or unsubstituted aryl, substituted or unsubstituted heteroaryl, or substituted or unsubstituted heterocyclic, where R is hydrogen, acyl, aliphatic or substituted aliphatic.

A cleavable group is one which is sufficiently stable outside the cell, but which upon entry into a target cell is cleaved to release the two parts the group is holding together. In a preferred embodiment, the cleavable group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable group by reduction; esterases; endosomes or agents that can create an acidic environment, e.g., those that result in a pH of five or lower; enzymes that can hydrolyze or degrade an acid cleavable group by acting as a general acid, peptidases (which can be substrate specific), and phosphatases.

A cleavable group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the cationic lipid from the ligand inside the cell, or into the desired compartment of the cell.

A conjugate can include a cleavable group that is cleavable by a particular enzyme. The type of cleavable group incorporated into a conjugate can depend on the cell to be targeted. For example, liver targeting ligands can be attached to the cationic lipids through a chemical moiety that includes an ester group. Liver cells are rich in esterases, and therefore the group will be cleaved more efficiently in liver cells than in cell types that are not esterase-rich. Other cell-types rich in esterases include cells of the lung, renal cortex, and testis.

Coupling groups that contain peptide bonds can be used when targeting cell types rich in peptidases, such as liver cells and synoviocytes.

In general, the suitability of a candidate cleavable group can be evaluated by testing the ability of a degradative agent (or condition) to cleave the candidate group. It will also be desirable to also test the candidate cleavable group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

Redox Cleavable Groups. One class of cleavable groups are redox cleavable groups that are cleaved upon reduction or oxidation. An example of reductively cleavable group is a disulphide linking group (—S—S—). To determine if a candidate cleavable group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-Based Cleavable Groups. Phosphate-based cleavable groups are cleaved by agents that degrade or hydrolyze the phosphate group. An example of an agent that cleaves phosphate groups in cells are enzymes such as phosphatases in cells. In some embodiments, the phosphate-based linking group is —O—P(═O)(OR^{k})—O—, —O—P(═S)(OR^{k})—O—, —O—P(═S)(SR^{k})—O—, —S—P(═O)(OR^{k})—O—, —O—P(═O)(OR^{k})—S—, —S—P(═O)(OR^{k})—S—, —O—P(═S)(OR^{k})-s-, —S—P(═S)(OR^{k})—O—, —O—P(═O)(R^{k})—, —O—P(═S)(R^{k})—O—, —S—P(═O)(R^{k})—O—, —S—P(═S)(R^{k})—, —S—P(═O)(R^{k})—S—, or —O—P(═S)(R^{k})—S—. In some embodiments, the phosphate-based linking group is —O—P(═O)(OH)—O—, —O—P(═S)(OH)—O—, —O—P(═S)(SH)—O—, —S—P(═O)(OH)—O—, —O—P(═O)(OH)—S—, —S—P(═O)(OH)—S—, —O—P(═S)(OH)—S—, —S—P(═S)(OH)—O—, —O—P(═O)(H)—O—, —O—P(═S)(H)—O—, —S—P(═O)(H)—O—, —S—P(═S)(H)—O—, —S—P(═O)(H)—S—, or —O—P(═S)(H)—S—. In some embodiments, the phosphate-based linking group is —O—P(═O)(OH)—O—.

Acid Cleavable Groups. Acid cleavable groups are linking groups that are cleaved under acidic conditions. In preferred embodiments acid cleavable groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-Based Cleavable Groups. Ester-based cleavable groups are cleaved by enzymes such as esterases and amidases in cells. Examples of ester-based cleavable groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-Based Cleavable Groups. Peptide-based cleavable groups are cleaved by enzymes such as peptidases and proteases in cells. Peptide-based cleavable groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids. These candidates can be evaluated using methods analogous to those described above. As used herein, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C₅ and above (preferably C₅-C₈) sugars; di- and trisaccharides include sugars having two or three monosaccharide units (preferably C₅-C₈).

### Methods of Synthesis

In some aspects, the present disclosure provides a method of preparing a compound (e.g., a Nucleotide-Based Enhancement Agent) described herein.

In some aspects, the present disclosure provides a compound obtainable by, or obtained by, a method for preparing a compound (e.g., a Nucleotide-Based Enhancement Agent) described herein.

In some aspects, the present disclosure provides an intermediate as described herein, being suitable for use in a method for preparing a compound (e.g., a Nucleotide-Based Enhancement Agent) described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. A suitable protecting group for an hydroxy or alkylhydroxy group can be, e.g., Acetyl (Ac), Benzoyl (Bz), Benzyl (Bn), β-Methoxyethoxymethyl ether (MEM), Dimethoxytrityl (DMT), Methoxymethyl ether (MOM), Methoxytrityl (MMT), p-Methoxybenzyl ether (PMB), p-Methoxyphenyl ether (PMP), Pivaloyl (Piv), Tetrahydropyranyl (THP), Tetrahydrofuran (THF), Trityl (triphenylmethyl, Tr), Silyl ether (e.g., trimethylsilyl (TMS), tert-butyldimethylsilyl (TBDMS), tri-iso-propylsilyloxymethyl (TOM), and triisopropylsilyl (TIPS) ethers), a Methyl ether, or an Ethoxyethyl ether (EE). A suitable protecting group for an 1,2-diol can be, e.g., acetal. A suitable protecting group for an 1,3-diol can be, e.g., tetraisopropyl-disiloxanylidene (TIPDS).

The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Scheme A herein.

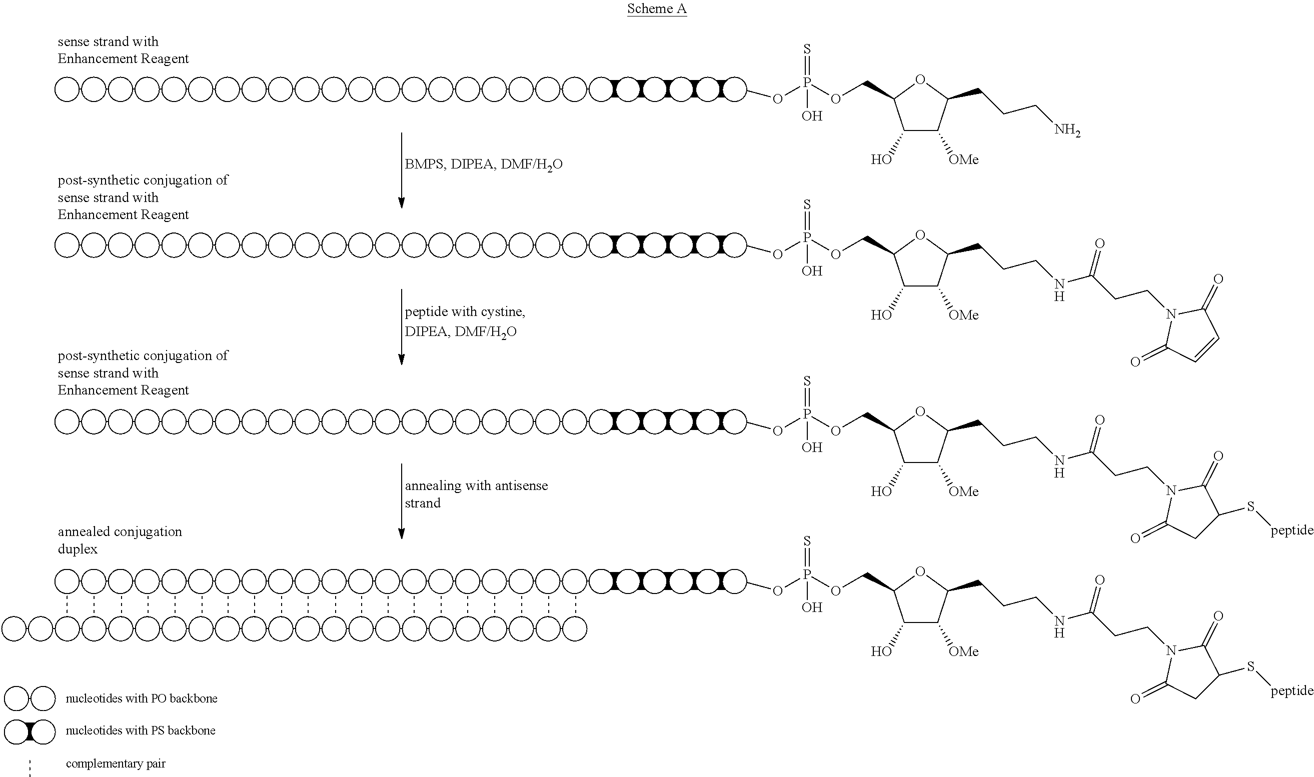

Compounds (e.g., Nucleic Acid Agents, Nucleic Acid Agents containing Nucleotide-Based Enhancement Agents, and Conjugates) were prepared by solid-phase synthesis according to standard synthesis protocols.

Briefly, oligonucleotide synthesis was conducted on a solid support to incorporate each nucleoside phosphoramidites from 3′-end to 5′-end to prepare oligo single strands. ETT or BTT was used as an activator for the coupling reaction. Iodine in water/pyridine-THF was used to oxidize phosphite-triester (P(III)) to afford phosphate backbones and DDTT was used for the preparation of phosphorothioate linkages. Aqueous ammonium was used to cleave oligos from solid support and to remove protecting groups globally. The oligo crude was then concentrated and purified by strong anion exchange or reverse phase HPLC. The purification fractions were combined and concentrated.

In some cases, oligo single strands were then conjugated with targeting ligands (e.g., peptides, antibodies) through post-synthetic conjugation to afford conjugates. The conjugation reactions were performed using standard conjugation methods. The conjugate crude was further purified by strong anion exchange or reverse phase HPLC. The purification fractions were combined and concentrated.

The synthesized single strands were then dialyzed against water using MidiTrap G-25 column, concentrated, and their OD amounts were measured. Based on the equal molar amounts, the sense and antisense strands were annealed at 95° C. for 5 min and cooled down to room temperature to afford the conjugate duplex with >90% purity. The solution of the duplex was lyophilized to afford the desired Conjugate and its amount was calculated based on the molar amount of single strand consumed in the annealing.

### Biological Assays

Compounds (e.g., Nucleotide-Based Enhancement Agents) or conjugates designed, selected, prepared and/or optimized by methods described above, once produced, can be characterized using a variety of assays known to those skilled in the art to determine whether the compounds, scaffolds, or conjugates have biological activity. For example, the compounds, scaffolds, or conjugates can be characterized by conventional assays, including but not limited to those assays described below, to determine whether they have a desired activity, e.g., target binding activity and/or specificity and/or stability.

Furthermore, high-throughput screening can be used to speed up analysis using such assays. As a result, it may be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds, scaffolds, or conjugates of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assays are described in the Examples herein.

In some aspects, the present disclosure provides a pharmaceutical composition comprising a compound, scaffold, or conjugate of the present disclosure as an active ingredient.

As used herein, the term “composition” is intended to encompass a product comprising the specified ingredients in the specified amounts, as well as any product which results, directly or indirectly, from combination of the specified ingredients in the specified amounts.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β3-cyclodextrin, methyl-3-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an inflammasome related condition referred to herein, slow its progression and/or reduce the symptoms associated with the condition.

An effective amount of a compound of the present disclosure for use in therapy is an amount sufficient to treat an inflammasome related condition referred to herein, slow its progression and/or reduce the symptoms associated with the condition.

The size of the dose for therapeutic or prophylactic purposes of a compound of Formula (I) or (II) will naturally vary according to the nature and severity of the conditions, the age and sex of the animal or patient and the route of administration, according to well-known principles of medicine.

### Methods of Use

In some aspects, the present disclosure provides a method of modulating (e.g., reducing or eliminating) the expression of a target gene in a subject, comprising administering to the subject a conjugate of the present disclosure.

In some aspects, the present disclosure provides a method of modulating (e.g., reducing or eliminating) the expression of a target gene in a cell or tissue of a subject, comprising administering to the subject a conjugate of the present disclosure.

In some aspects, the present disclosure provides a method of delivering a Nucleic Acid Agent to a subject, comprising administering to the subject a conjugate of the present disclosure.

In some aspects, the present disclosure provides a method of treating or preventing a disease in a subject in need thereof, comprising administering to the subject a therapeutically effective amount of a conjugate of the present disclosure.

In some aspects, the present disclosure provides a conjugate of the present disclosure for modulating (e.g., reducing or eliminating) the expression of a target gene in a subject.

In some aspects, the present disclosure provides a conjugate of the present disclosure for modulating (e.g., reducing or eliminating) the expression of a target gene in a cell or tissue of a subject.

In some aspects, the present disclosure provides a conjugate of the present disclosure for delivering a Nucleic Acid Agent to a subject.

In some aspects, the present disclosure provides a conjugate of the present disclosure for treating or preventing a disease in a subject in need thereof.

In some aspects, the present disclosure provides use of a conjugate of the present disclosure in the manufacture of a medicament for modulating (e.g., reducing or eliminating) the expression of a target gene in a subject.

In some aspects, the present disclosure provides use of a conjugate of the present disclosure in the manufacture of a medicament for modulating (e.g., reducing or eliminating) the expression of a target gene in a cell or tissue of a subject.

In some aspects, the present disclosure provides use of a conjugate of the present disclosure in the manufacture of a medicament for delivering a Nucleic Acid Agent to a subject.

In some aspects, the present disclosure provides use of a conjugate of the present disclosure in the manufacture of a medicament for treating or preventing a disease in a subject in need thereof.

In some embodiments, the subject is a cell.

In some embodiments, the subject is a central nervous system, peripheral nervous system, adipose, muscle, heart, and/or pancreas cell. In some embodiments, the subject is a pancreatic cell. In some embodiments, the subject is a pancreatic beta cell.

In some embodiments, the subject is a tissue.

In some embodiments, the subject is a central nervous system, peripheral nervous system, adipose, muscle, heart, and/or pancreas tissue. In some embodiments, the subject is a pancreas tissue.

In some embodiments, the subject is a human.

In some embodiments, the target gene is Factor VII, Eg5, PCSK9, TPX2, apoB, SAA, TTR, HBV, HCV, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase 11 alpha gene, p73 gene, p21(WAF1/CIP1) gene, p27(KIP1) gene, PPM1D gene, RAS gene, caveolin I gene, MIB 1 gene, MTAI gene, M68 gene, mutations in tumor suppressor genes, p53 tumor suppressor gene, LDHA, or any combination thereof.

In some embodiments, the disease characterized by unwanted expression of the target gene.

In some embodiments, the administration results in reduced or eliminated expression of the target gene in the subject.

In some embodiments, the disease is a viral infection, e.g., an HCV, HBV, HPV, HSV or HIV infection.

In some embodiments, the disease is cancer.

In some embodiments, the cancer is biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, lung cancer, non-small cell lung cancer (NSCLC), mesothelioma, B-cell lymphomas, non-Hodgkin lymphoma, diffuse large B-cell lymphoma, Mantle cell lymphoma, T-cell lymphomas, non-Hodgkin lymphoma, precursor T-lymphoblastic lymphoma/leukemia, peripheral T-cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

In some embodiments, the cancer is liver cancer, liver carcinoma, hepatoma, hepatocellular carcinoma, cholangiocarcinoma, or hepatoblastoma.

In some embodiments, the disease is a proliferative, inflammatory, autoimmune, neurologic, ocular, respiratory, metabolic, dermatological, auditory, liver, kidney, or infectious disease. In some embodiments, the disease is a disease of the liver.

In some embodiments, the disease is a disease of the central nervous system, peripheral nervous system, adipose, muscle, heart, and/or pancreas. In some embodiments, the disease is a disease of the pancreas.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently attached to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “overhang” refers to the section of unpaired nucleotide(s) at the end of an oligonucleotide strand (e.g. the 3′-end or 5′end). Overhang at the 3′-end of an oligonucleotide strand is the “3′ overhang,” and overhang at the 5′-end of an oligonucleotide strand is the “5′ overhang.”

As used herein, the expressions “one or more of A, B, or C.” “one or more A, B, or C,” “one or more of A, B, and C.” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds, scaffolds, and conjugates described herein. The present disclosure also provides detailed methods for the synthesis of various disclosed compounds, scaffolds, and conjugates according to the schemes herein as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds, scaffolds, and conjugates of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition. John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds, scaffolds, and conjugates to provide such treatment or prevention as is described herein. It is to be further understood, unless otherwise stated, any description of a method of treatment or prevention includes use of the compounds, scaffolds, and conjugates to prepare a medicament to treat or prevent such condition. The treatment or prevention includes treatment or prevention of human or non-human animals including rodents and other disease models.

It is to be understood that, unless otherwise stated, any description of a method of treatment includes use of the compounds, scaffolds, and conjugates to provide such treatment as is described herein. It is to be further understood, unless otherwise stated, any description of a method of treatment includes use of the compounds, scaffolds, and conjugates to prepare a medicament to treat such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that compounds, scaffolds, and conjugates of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that the present disclosure also provides pharmaceutical compositions comprising any compound, scaffold, or conjugate described herein in combination with at least one pharmaceutically acceptable excipient or carrier.

As used herein, the term “pharmaceutical composition” is a formulation containing the compounds, scaffolds, or conjugates of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

As used herein, the term “pharmaceutically acceptable” refers to those compounds, scaffolds, conjugates, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebuliser.

For intranasal administration, the compounds are delivered in solution or solid formulation. In some embodiments, the compounds are delivered in solution as a mist, a drip, or a swab. In some embodiments, the compounds are delivered as a powder. In some embodiments, the compound is included in a kit which further includes an intranasal applicator.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds, scaffolds, or conjugates of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1.3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy., 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXEMPLARY EMBODIMENTS

Exemplary Embodiment No. 1. A Nucleotide-Based Enhancement Agent being a compound or a pharmaceutically acceptable salt thereof, comprising from 2 to 30 nucleotides, wherein each nucleotide independently is of Formula (I) or (II):

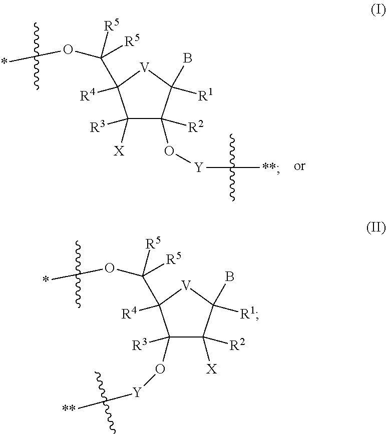

wherein:
each * independently denotes an attachment to the 2′- or 3′-position of another nucleotide of the Nucleotide-Based Enhancement Agent, or denotes H when the nucleotide is at the 5′-end of the Nucleotide-Based Enhancement Agent;
each ** independently denotes an attachment to the 5′-position of another nucleotide of the Nucleotide-Based Enhancement Agent, or denotes H when the nucleotide is at the 2′- or 3′-end of the Nucleotide-Based Enhancement Agent;
B is H, C₁-C₆ alkyl, or a nucleobase moiety;
V is —O—, —NR^{V}—, or —C(R^{V})₂—;
each R^{Y} independently is H or C₁-C₆ alkyl;
X is H, halogen, or —OR^{X};
R^{X} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Xa}; or R^{X} and R⁴ together form C₁-C₆ alkylene;
each R^{Xa} independently is halogen, C₁-C₆ alkyl, or —O—(C₁-C₆ alkyl), wherein the C₁-C₆ alkyl or —O—(C₁-C₆ alkyl) is optionally substituted with one or more halogen;
Y is —P(R^{Y})—, —P(OR^{Y})—, —P(N(R^{Y})₂)—, —P(═O)(OR^{Y})—, —P(═O)(R^{Y})—, —P(═S)(OR^{Y})—, —P(═S)(R^{Y})—, —P(═O)(SR^{Y})—, or —P(═S(SR^{Y})—;
each R^{Y} independently is H or C₁-C₆ alkyl optionally substituted with one or more halogen or cyano;
R¹ is H, halogen, or C₁-C₆ alkyl optionally substituted with one or more halogen;
R² is H, halogen, or C₁-C₆ alkyl optionally substituted with one or more halogen;
R³ is H, halogen, or C₁-C₆ alkyl optionally substituted with one or more halogen;
R⁴ is H, halogen, or C₁-C₆ alkyl optionally substituted with one or more halogen; or R⁴ and R^{X} together form C₁-C₆ alkylene; and
each R⁵ independently is H, halogen, or C₁-C₆ alkyl optionally substituted with one or more halogen.

Exemplary Embodiment No. 2. A conjugate or a pharmaceutically acceptable salt thereof, comprising:
(i) one or more Nucleic Acid Agent;
(ii) one or more Ligand; and
(iii) one or more Nucleotide-Based Enhancement Unit, wherein each Nucleotide-Based Enhancement Unit independently comprises from 2 to 30 nucleotides, wherein each nucleotide independently is of;

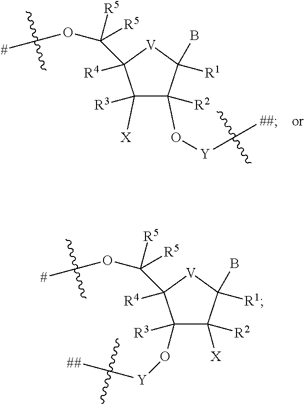

wherein:
variables B, V, X, Y, R¹, R², R³, R⁴, and R⁵ are described herein;
each # independently denotes an attachment to the 2′- or 3′-position of another nucleotide of the Nucleotide-Based Enhancement Unit, or denotes H or an attachment to the rest of the conjugate when the nucleotide is at the 5′-end of the Nucleotide-Based Enhancement Unit; and
each ## independently denotes an attachment to the 5′-position of another nucleotide of the Nucleotide-Based Enhancement Unit, or denotes H or an attachment to the rest of the conjugate when the nucleotide is at the 2′- or 3-end of the Nucleotide-Based Enhancement Unit.

Exemplary Embodiment No. 3. The Nucleotide-Based Enhancement Agent or conjugate of any one of the preceding Exemplary Embodiments, wherein B is H.

Exemplary Embodiment No. 4. The Nucleotide-Based Enhancement Agent or conjugate of any one of the preceding Exemplary Embodiments, wherein B is a nucleobase moiety.

Exemplary Embodiment No. 5. The Nucleotide-Based Enhancement Agent or conjugate of any one of the preceding Exemplary Embodiments, wherein the nucleobase moiety is adenine (A), cytosine (C), guanine (G), thymine (T), or uracil (U).

Exemplary Embodiment No. 6. The Nucleotide-Based Enhancement Agent or conjugate of any one of the preceding Exemplary Embodiments, wherein the nucleobase moiety is a modified nucleobase.

Exemplary Embodiment No. 7. The Nucleotide-Based Enhancement Agent or conjugate of any one of the preceding Exemplary Embodiments, wherein the nucleobase moiety is an artificial nucleobase.

Exemplary Embodiment No. 8. The Nucleotide-Based Enhancement Agent or conjugate of any one of the preceding Exemplary Embodiments, wherein V is —O—.

Exemplary Embodiment No. 9. The Nucleotide-Based Enhancement Agent or conjugate of any one of the preceding Exemplary Embodiments, wherein V is —NR^{V}.

Exemplary Embodiment No. 10. The Nucleotide-Based Enhancement Agent or conjugate of any one of the preceding Exemplary Embodiments, wherein V is —NH—.

Exemplary Embodiment No. 11. The Nucleotide-Based Enhancement Agent or conjugate of any one of the preceding Exemplary Embodiments, wherein V is —C(R^{V})₂—.

Exemplary Embodiment No. 12. The Nucleotide-Based Enhancement Agent or conjugate of any one of the preceding Exemplary Embodiments, wherein V is —CH₂—.

Exemplary Embodiment No. 13. The Nucleotide-Based Enhancement Agent or conjugate of any one of the preceding Exemplary Embodiments, wherein X is H.

Exemplary Embodiment No. 14. The Nucleotide-Based Enhancement Agent or conjugate of any one of the preceding Exemplary Embodiments, wherein X is halogen.

Exemplary Embodiment No. 15. The Nucleotide-Based Enhancement Agent or conjugate of any one of the preceding Exemplary Embodiments, wherein X is —OR^{X}.

Exemplary Embodiment No. 16. The Nucleotide-Based Enhancement Agent or conjugate of any one of the preceding Exemplary Embodiments, wherein X is —OH.

Exemplary Embodiment No. 17. The Nucleotide-Based Enhancement Agent or conjugate of any one of the preceding Exemplary Embodiments, wherein X is —O—(C₁-C₆ alkyl).

Exemplary Embodiment No. 18. The Nucleotide-Based Enhancement Agent or conjugate of any one of the preceding Exemplary Embodiments, wherein X is —O—(C₁-C₆ alkyl)-O—(C₁-C₆ alkyl).

Exemplary Embodiment No. 19. The Nucleotide-Based Enhancement Agent or conjugate of any one of the preceding Exemplary Embodiments, wherein X is —O—(C₁-C₆ alkyl)-(C₆-C₁₀ aryl) optionally substituted with one or more R^{X}.

Exemplary Embodiment No. 20. The Nucleotide-Based Enhancement Agent or conjugate of any one of the preceding Exemplary Embodiments, wherein R^{X} is H.

Exemplary Embodiment No. 21. The Nucleotide-Based Enhancement Agent or conjugate of any one of the preceding Exemplary Embodiments, wherein R^{X} is C₁-C₆ alkyl optionally substituted with one or more R^{Xa}.

Exemplary Embodiment No. 22. The Nucleotide-Based Enhancement Agent or conjugate of any one of the preceding Exemplary Embodiments, wherein R^{X} is —(C₁-C₆ alkyl)-(C₆-C₁₀ aryl) optionally substituted with one or more R^{Xa}.

Exemplary Embodiment No. 23. The Nucleotide-Based Enhancement Agent or conjugate of any one of the preceding Exemplary Embodiments, wherein R^{X} is —(C₁-C₆ alkyl)-(C₆-C₁₀ aryl).

Exemplary Embodiment No. 24. The Nucleotide-Based Enhancement Agent or conjugate of any one of the preceding Exemplary Embodiments, wherein R^{X} and R⁴ together form C₁-C₆ alkylene.

Exemplary Embodiment No. 25. The Nucleotide-Based Enhancement Agent or conjugate of any one of the preceding Exemplary Embodiments, wherein Y is —P(R^{Y})—.

Exemplary Embodiment No. 26. The Nucleotide-Based Enhancement Agent or conjugate of any one of the preceding Exemplary Embodiments, wherein Y is —P(OR^{Y})—.

Exemplary Embodiment No. 27. The Nucleotide-Based Enhancement Agent or conjugate of any one of the preceding Exemplary Embodiments, wherein Y is —P(N(R^{Y})₂)—.

Exemplary Embodiment No. 28. The Nucleotide-Based Enhancement Agent or conjugate of any one of the preceding Exemplary Embodiments, wherein Y is —P(═O)(OR^{Y})—.

Exemplary Embodiment No. 29. The Nucleotide-Based Enhancement Agent or conjugate of any one of the preceding Exemplary Embodiments, wherein Y is —P(═O)(R^{Y})—.

Exemplary Embodiment No. 30. The Nucleotide-Based Enhancement Agent or conjugate of any one of the preceding Exemplary Embodiments, wherein Y is —P(═S)(OR^{Y})—.

Exemplary Embodiment No. 31. The Nucleotide-Based Enhancement Agent or conjugate of any one of the preceding Exemplary Embodiments, wherein Y is —P(═S)(R^{Y})—.

Exemplary Embodiment No. 32. The Nucleotide-Based Enhancement Agent or conjugate of any one of the preceding Exemplary Embodiments, wherein Y is —P(═O)(SR^{Y})—.

Exemplary Embodiment No. 33. The Nucleotide-Based Enhancement Agent or conjugate of any one of the preceding Exemplary Embodiments, wherein Y is —P(═S)(SR^{Y})—.

Exemplary Embodiment No. 34. The Nucleotide-Based Enhancement Agent or conjugate of any one of the preceding Exemplary Embodiments, wherein each R^{Y} is H.

Exemplary Embodiment No. 35. The Nucleotide-Based Enhancement Agent or conjugate of any one of the preceding Exemplary Embodiments, wherein at least one R^{Y} is C₁-C₆ alkyl optionally substituted with one or more halogen or cyano.

Exemplary Embodiment No. 36. The Nucleotide-Based Enhancement Agent or conjugate of any one of the preceding Exemplary Embodiments, wherein each R^{Y} is C₁-C₆ alkyl optionally substituted with one or more halogen or cyano.

Exemplary Embodiment No. 37. The Nucleotide-Based Enhancement Agent or conjugate of any one of the preceding Exemplary Embodiments, wherein R¹ is H.

Exemplary Embodiment No. 38. The Nucleotide-Based Enhancement Agent or conjugate of any one of the preceding Exemplary Embodiments, wherein R¹ is halogen.

Exemplary Embodiment No. 39. The Nucleotide-Based Enhancement Agent or conjugate of any one of the preceding Exemplary Embodiments, wherein R¹ is C₁-C₆ alkyl optionally substituted with one or more halogen.

Exemplary Embodiment No. 40. The Nucleotide-Based Enhancement Agent or conjugate of any one of the preceding Exemplary Embodiments, wherein R² is H.

Exemplary Embodiment No. 41. The Nucleotide-Based Enhancement Agent or conjugate of any one of the preceding Exemplary Embodiments, wherein R² is halogen.

Exemplary Embodiment No. 42. The Nucleotide-Based Enhancement Agent or conjugate of any one of the preceding Exemplary Embodiments, wherein R² is C₁-C₆ alkyl optionally substituted with one or more halogen.

Exemplary Embodiment No. 43. The Nucleotide-Based Enhancement Agent or conjugate of any one of the preceding Exemplary Embodiments, wherein R³ is H.

Exemplary Embodiment No. 44. The Nucleotide-Based Enhancement Agent or conjugate of any one of the preceding Exemplary Embodiments, wherein R³ is halogen.

Exemplary Embodiment No. 45. The Nucleotide-Based Enhancement Agent or conjugate of any one of the preceding Exemplary Embodiments, wherein R³ is C₁-C₆ alkyl optionally substituted with one or more halogen.

Exemplary Embodiment No. 46. The Nucleotide-Based Enhancement Agent or conjugate of any one of the preceding Exemplary Embodiments, wherein R⁴ is H.

Exemplary Embodiment No. 47. The Nucleotide-Based Enhancement Agent or conjugate of any one of the preceding Exemplary Embodiments, wherein R⁴ is halogen.

Exemplary Embodiment No. 48. The Nucleotide-Based Enhancement Agent or conjugate of any one of the preceding Exemplary Embodiments, wherein R⁴ is C₁-C₆ alkyl optionally substituted with one or more halogen.

Exemplary Embodiment No. 49. The Nucleotide-Based Enhancement Agent or conjugate of any one of the preceding Exemplary Embodiments, wherein R⁴ and R^{X} together form C₁-C₆ alkylene.

Exemplary Embodiment No. 50. The Nucleotide-Based Enhancement Agent or conjugate of any one of the preceding Exemplary Embodiments, wherein each R⁵ is H.

Exemplary Embodiment No. 51. The Nucleotide-Based Enhancement Agent or conjugate of any one of the preceding Exemplary Embodiments, wherein at least one R⁵ is halogen.

Exemplary Embodiment No. 52. The Nucleotide-Based Enhancement Agent or conjugate of any one of the preceding Exemplary Embodiments, wherein at least one R⁵ is C₁-C₆ alkyl optionally substituted with one or more halogen.

Exemplary Embodiment No. 53. The Nucleotide-Based Enhancement Agent or conjugate of any one of the preceding Exemplary Embodiments, wherein each of R^{a}, R^{b}, R¹, R², R³, R⁴, and R⁵ is H.

Exemplary Embodiment No. 54. The Nucleotide-Based Enhancement Agent of any one of the preceding Exemplary Embodiments, wherein at least one nucleotide of the Nucleotide-Based Enhancement Agent is of Formula (I′) or (II′):

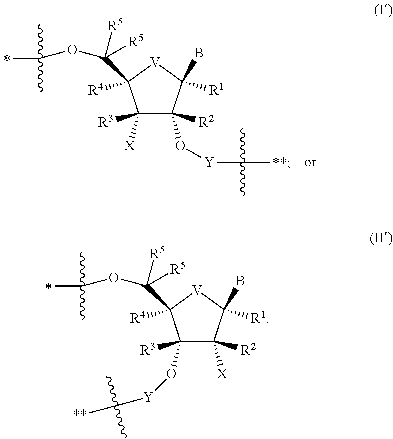

Exemplary Embodiment No. 55. The Nucleotide-Based Enhancement Agent of any one of the preceding Exemplary Embodiments, wherein at least one nucleotide of the Nucleotide-Based Enhancement Agent is of Formula (I-A), (II-A), (III-A), or (TV-A):

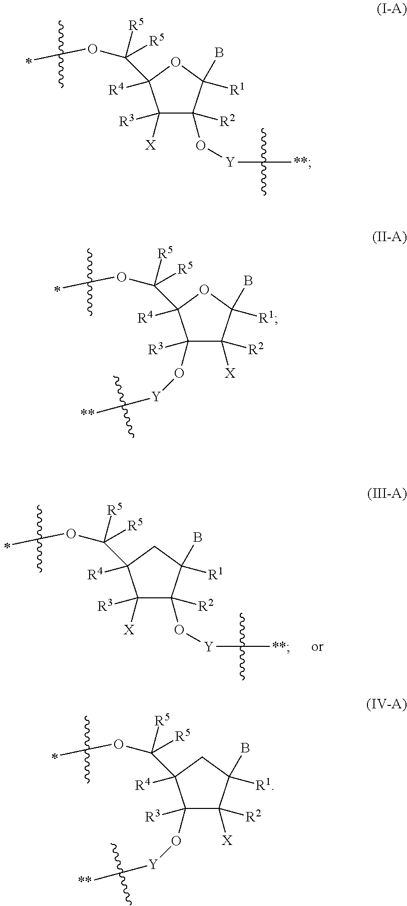

Exemplary Embodiment No. 56. The Nucleotide-Based Enhancement Agent of any one of the preceding Exemplary Embodiments, wherein at least one nucleotide of the Nucleotide-Based Enhancement Agent is of Formula (I′-A), (II′-A), (III′-A), or (II′-A):

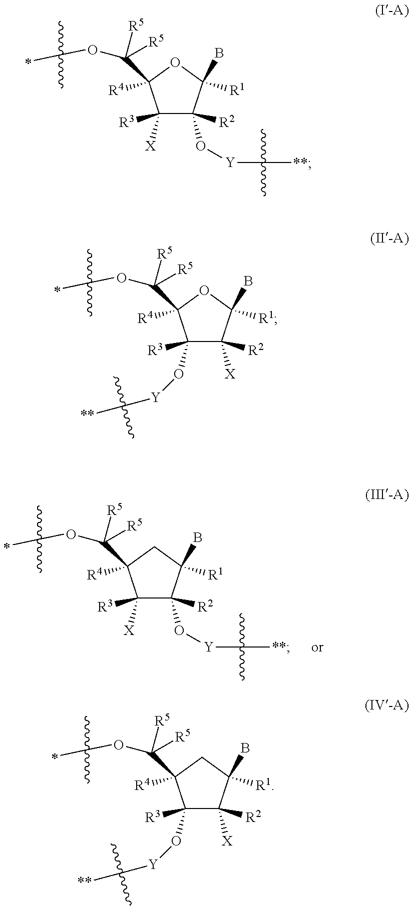

Exemplary Embodiment No. 57. The Nucleotide-Based Enhancement Agent of any one of the preceding Exemplary Embodiments, being selected from the compounds described in Table L and pharmaceutically acceptable salts thereof.

Exemplary Embodiment No. 58. The Nucleotide-Based Enhancement Agent of any one of the preceding Exemplary Embodiments, wherein at least one nucleotide of the Nucleotide-Based Enhancement Agent is of Formula (I-B) or (II-B):

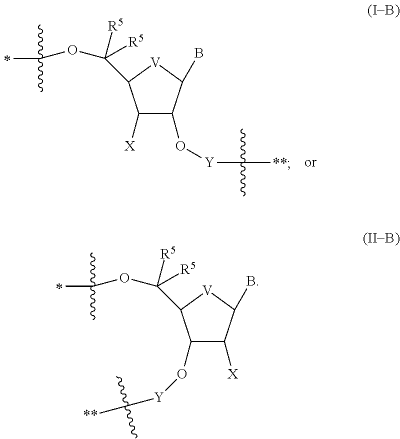

Exemplary Embodiment No. 59. The Nucleotide-Based Enhancement Agent of any one of the preceding Exemplary Embodiments, wherein at least one nucleotide of the Nucleotide-Based Enhancement Agent is of Formula (I′-B) or (II′-B):

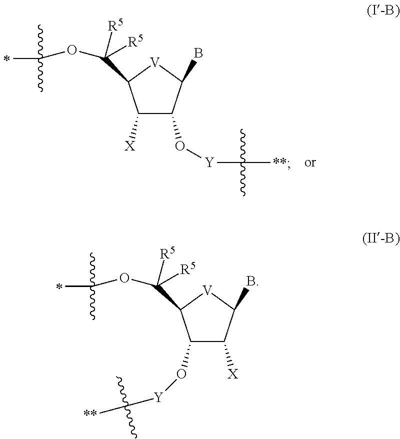

Exemplary Embodiment No. 60. The Nucleotide-Based Enhancement Agent of any one of the preceding Exemplary Embodiments, wherein at least one nucleotide of the Nucleotide-Based Enhancement Agent is of Formula (I-C), (II-C), (III-C), (IV-C):

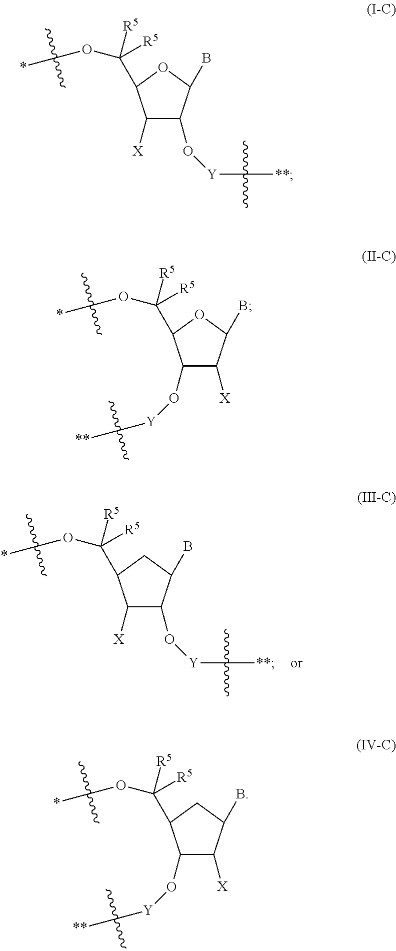

Exemplary Embodiment No. 61. The Nucleotide-Based Enhancement Agent of any one of the preceding Exemplary Embodiments, wherein at least one nucleotide of the Nucleotide-Based Enhancement Agent is of Formula (I′-C), (II′-C), (III′-C), or (IV′-C):

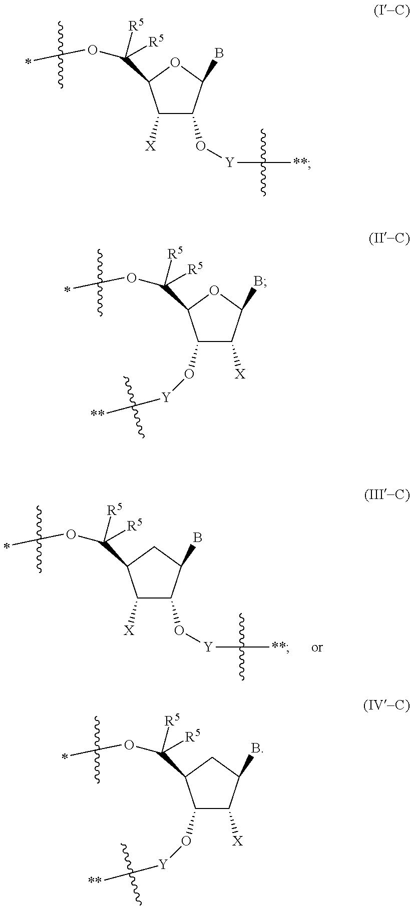

Exemplary Embodiment No. 62. The Nucleotide-Based Enhancement Agent of any one of the preceding Exemplary Embodiments, wherein at least one nucleotide of the Nucleotide-Based Enhancement Agent is selected from the nucleotides described in Table L.

Exemplary Embodiment No. 63. An isotopic derivative of the Nucleotide-Based Enhancement Agent of any one of the preceding Exemplary Embodiments.

Exemplary Embodiment No. 64. The conjugate of any one of the preceding Exemplary Embodiments, comprising a double strand RNA.

Exemplary Embodiment No. 65. The conjugate of any one of the preceding Exemplary Embodiments, being selected from the conjugates described in FIG. 1.

Exemplary Embodiment No. 66. The conjugate of any one of the preceding Exemplary Embodiments, being selected from the structures described in Table C.

Exemplary Embodiment No. 67. The conjugate of any one of the preceding Exemplary Embodiments, wherein the Nucleotide-Based Enhancement Unit corresponds to a Nucleotide-Based Enhancement Agent in which the * in the nucleotide is at the 5′-end is attached to a Ligand, a Linker Unit, and/or a Nucleic Acid.

Exemplary Embodiment No. 68. The conjugate of any one of the preceding Exemplary Embodiments, wherein the ligand comprises a carbohydrate moiety.

Exemplary Embodiment No. 69. The conjugate of any one of the preceding Exemplary Embodiments, wherein the carbohydrate moiety comprises a monosaccharide, a disaccharide, a trisaccharide, or a tetrasaccharide.

Exemplary Embodiment No. 70. The conjugate of any one of the preceding Exemplary Embodiments, wherein the carbohydrate moiety comprises galactose or a derivative thereof.

Exemplary Embodiment No. 71. The conjugate of any one of the preceding Exemplary Embodiments, wherein the ligand comprises

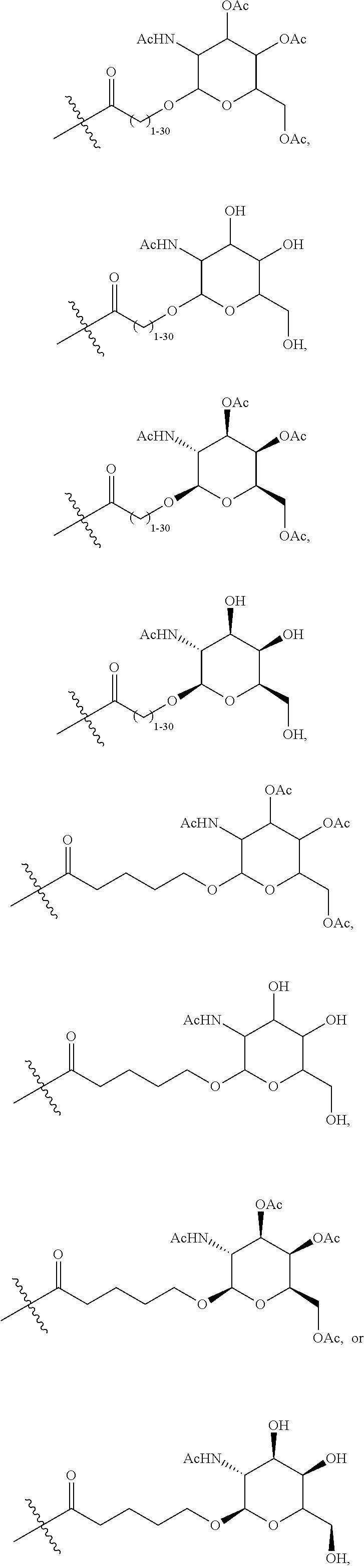

Exemplary Embodiment No. 72. The conjugate of any one of the preceding Exemplary Embodiments, wherein the ligand comprises

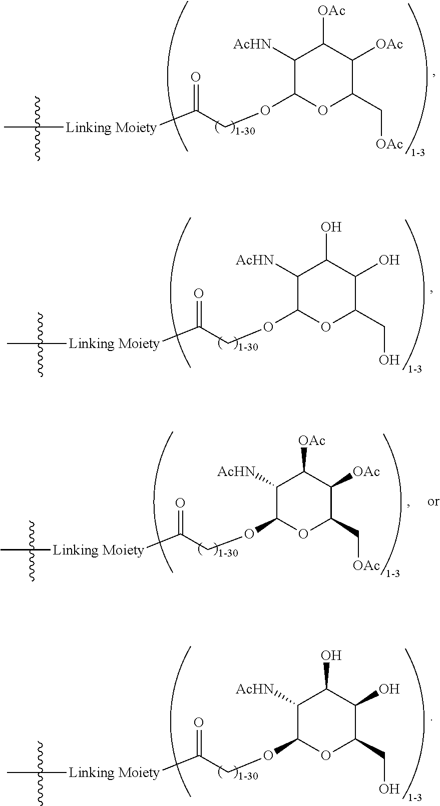

Exemplary Embodiment No. 73. The conjugate of any one of the preceding Exemplary Embodiments, wherein the ligand comprises

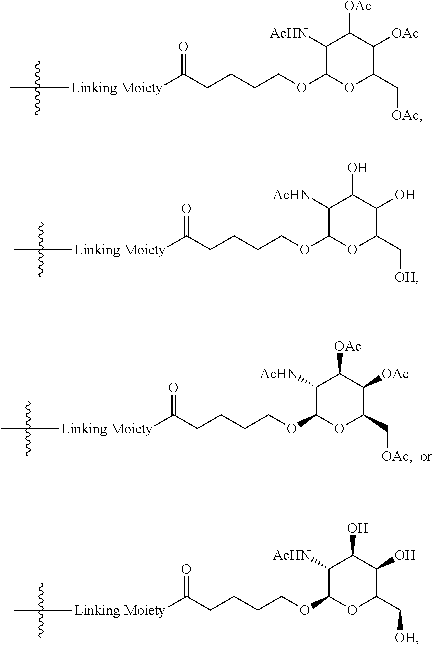

Exemplary Embodiment No. 74. The conjugate of any one of the preceding claims, wherein the ligand comprises a moiety capable of binding to a glucagon-like peptide-1 receptor (GLP-1 receptor).

Exemplary Embodiment No. 75. The conjugate of any one of the preceding claims, wherein the ligand comprises glucagon-like peptide-1 (GLP-1) or a derivative thereof.

Exemplary Embodiment No. 76. The conjugate of any one of the preceding claims, wherein the ligand comprises glucagon or a derivative thereof.

Exemplary Embodiment No. 77. The conjugate of any one of the preceding Exemplary Embodiments, wherein the ligand comprises:
one, two, or three sugar (e.g., N-acetyl-D-galactosylamine);
one, two, or three lipid moiety;
one, two, or three peptide moiety;
one, two, or three antibody moiety;
one, two, or three oligonucleotide; or
any combination thereof.

Exemplary Embodiment No. 78. A pharmaceutical composition comprising the Nucleotide-Based Enhancement Agent or conjugate of any one of the preceding Exemplary Embodiments.

Exemplary Embodiment No. 79. A method of modulating the expression of a target gene in a subject, comprising administering to the subject the conjugate of any one of the preceding Exemplary Embodiments.

Exemplary Embodiment No. 80. A method of delivering a Nucleic Acid Agent to a subject, comprising administering to the subject the conjugate of any one of the preceding Exemplary Embodiments.

Exemplary Embodiment No. 81. A method of treating or preventing a disease in a subject in need thereof, comprising administering to the subject a therapeutically effective amount of the conjugate of any one of the preceding Exemplary Embodiments.

Exemplary Embodiment No. 82. The conjugate of any one of the preceding Exemplary Embodiments for modulating the expression of a target gene in a subject.

Exemplary Embodiment No. 83. The conjugate of any one of the preceding Exemplary Embodiments for delivering a Nucleic Acid Agent to a subject.

Exemplary Embodiment No. 84. The conjugate of any one of the preceding Exemplary Embodiments for treating or preventing a disease in a subject in need thereof.

Exemplary Embodiment No. 85. Use of the conjugate of any one of the preceding Exemplary Embodiments in the manufacture of a medicament for modulating the expression of a target gene in a subject.

Exemplary Embodiment No. 86. Use of the conjugate of any one of the preceding Exemplary Embodiments in the manufacture of a medicament for delivering a Nucleic Acid Agent to a subject.

Exemplary Embodiment No. 87. Use of the conjugate of any one of the preceding Exemplary Embodiments in the manufacture of a medicament for treating or preventing a disease in a subject in need thereof.

Exemplary Embodiment No. 88. The method, conjugate, or use of any one of the preceding Exemplary Embodiments, wherein the subject is a human.

### EXAMPLES

### Example 1. Synthesis of Duplex 1-5

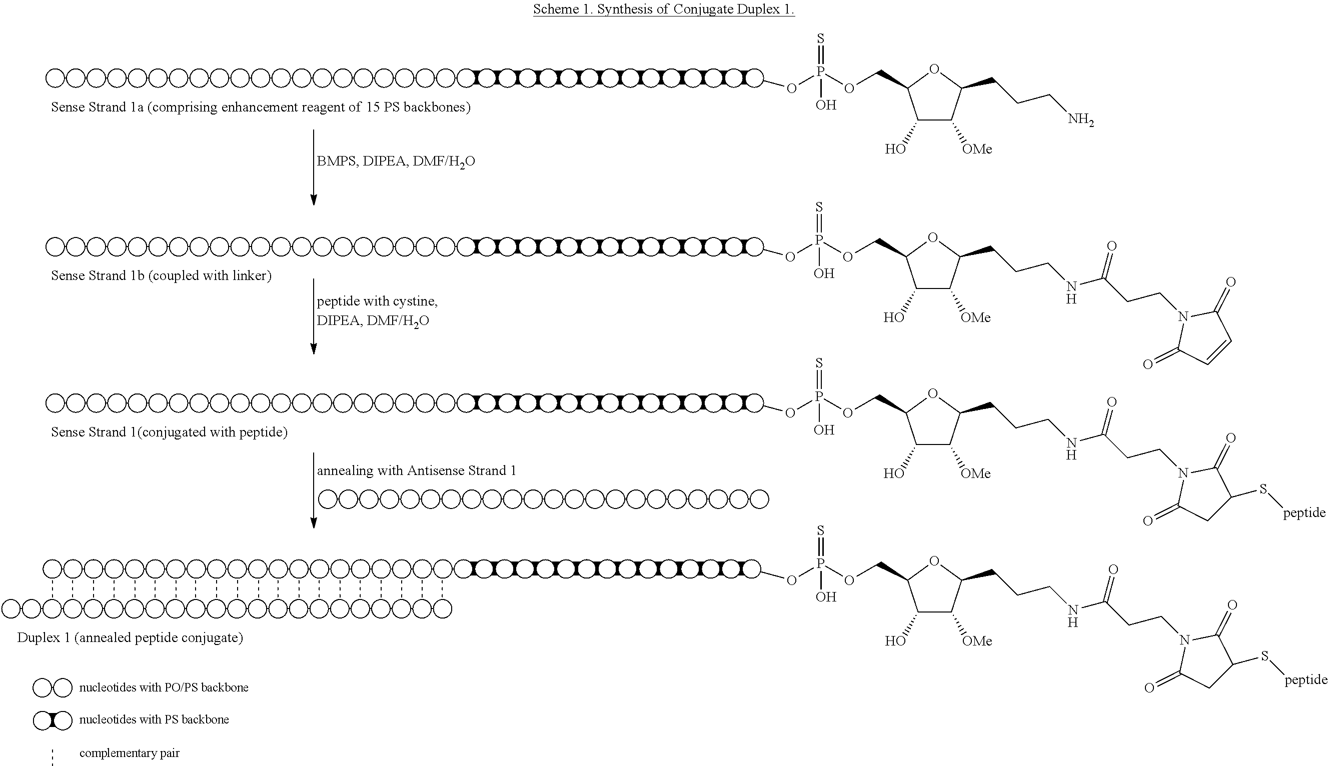

Duplex I was synthesized following Scheme 1. Sense Strand 1a and Antisense Strand 1 were prepared by solid-phase synthesis according to manufacturer's protocol.

Sense Strand 1b was generated via post-synthetic conjugation. To Eppendorf tube 1, Sense Strand 1a (10.00 mg, 0.71 umol) and H₂O (0.50 mL) were added and mixed vigorously to obtain a clear solution. To Eppendorf tube 2, DMF (0.50 mL), N-β-maleimidopropyl-oxysuccinimide ester (0.38 mg, 1.42 umol), and DIPEA (1.24 uL, 7.10 umol) were added and mixed vigorously. The DMF solution in Eppendorf tube 2 was then added to Eppendorf tube 1 and the resulting mixture was stirred at rt. After the reaction was completed indicated by LC-MS analysis, it was quenched with H₂O (10 mL). The reaction mixture was dialyzed against water (3×15 mL) using Amicon® Ultra-15 Centrifugal with MW cut-off of 3K. The solution was lyophilized to afford crude Sense Strand 1b (8.23 mg, 82% yield) that was used directly for the next step without further purification.

Sense Strand 1 was prepared via post-synthetic conjugation. To Eppendorf tube 1, Sense Strand 1b (8.23 mg, 0.58 umol) and H₂O (0.20 mL) were added and mixed vigorously to obtain a clear solution. To Eppendorf tube 2, DMA (0.60 mL), peptide (3.9 mg, 0.87 umol), and DIPEA (1.02 uL, 5.80 umol) were added and mixed vigorously. The DMA solution in Eppendorf tube 2 was then added to Eppendorf tube 1 and the resulting mixture was stirred at rt. After the reaction was completed indicated by LC-MS analysis, it was quenched with H₂O (2 mL). The reaction mixture was purified by reverse-phase HPLC (C18 column, 100 mM of TEAA in I and H₂O, 10-95% gradient, 30 min). Fractions were combined, dialyzed against saline (15 mL) and water (3×15 mL) using Amicon® Ultra-15 Centrifugal (3K) with MW cut-off of 3K. The solution was then lyophilized to afford a white solid of Sense Strand 1 (6.42 mg, 58% yield).

Duplex 1 was generated by annealing of Sense Strand 1 and Antisense 1. Sense Strand 1 solution (10 mg/mL) and Antisense Strand 1 solution (10 mg/mL) were prepared according to their OD amounts. Based on equal molar amounts, the sense and antisense strands were mixed and annealed at 95° C. for 5 min and cooled down to room temperature to afford the conjugate duplex with >90% purity. The solution of the duplex was lyophilized to afford the desired Duplex 1 (96% yield).

Duplex 2-5 (see FIG. 2) were prepared using similar procedures as described for the preparation of Duplex 1 with comparable yields and purity.

### Example 2. In Vivo KD Activity of Duplex 1-5

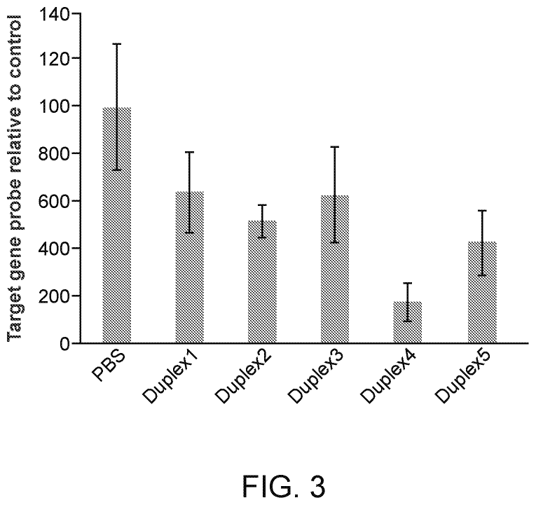

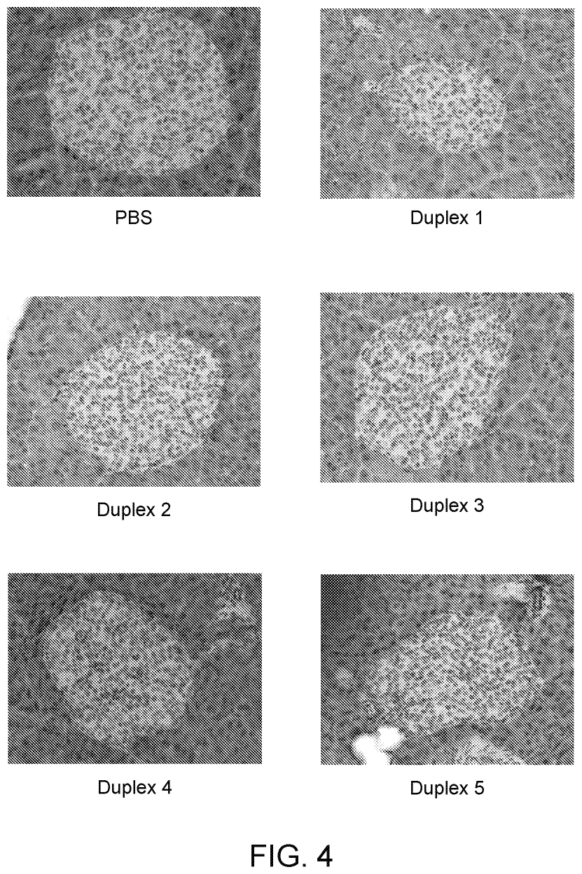

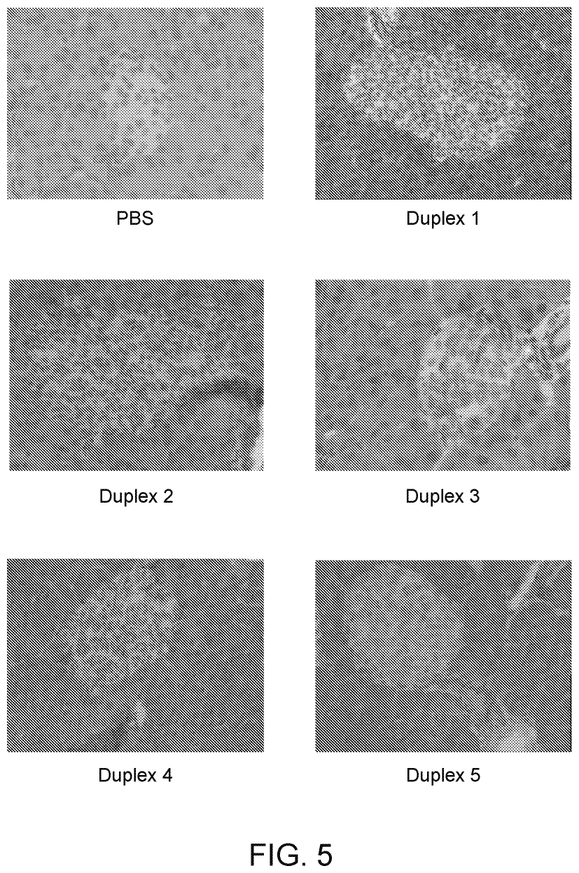

CD-1 female mice were administrated subcutaneously with Duplex 1-5 at 30 mg/kg. A control group was dosed with phosphate buffered saline (PBS). Animals were sacrificed 7 days post-treatment. Specific tissues of interests were collected and preserved in 10% for 24 hrs. The tissues were then transferred to 75% ethanol and embedded in paraffine blocks. 4 μm tissue sections were made from the paraffine blocks. The slides were deparaffined in 100% xylene twice for 5 min and 100% ethanol twice for 5 min. The RNASCOPE assay against the target gene was conducted following the RNASCOPE kit manual (RNASCOPE 2.5 HD assay-Red, CAT NO: 322350, Advanced Cell Diagnostics). The post-RNASCOPE slides were imaged with Keyence microscope and imageS were quantified with Indica Halo software. All samples were normalized to the PBS treated control samples and plotted using GraphPad Prism software (GraphPad Software Inc., La Jolla, CA). For the detailed results, see FIGS. 3-5.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A conjugate or a pharmaceutically acceptable salt thereof, comprising:
(i) one or more Nucleic Acid Agent;
(ii) one or more Ligand; and
(iii) one or more Nucleotide-Based Enhancement Unit, wherein each Nucleotide-Based Enhancement Unit independently comprises from 2 to 30 nucleotides, wherein each nucleotide independently is of: 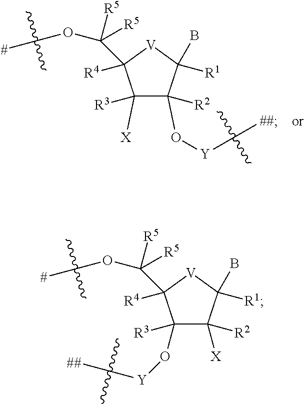
wherein:
Nucleic Acid Agent comprises an oligonucleotide;
Ligand is a moiety that, when covalently attached to the Nucleic Acid Agent, is capable of mediating its entry into, or facilitating its delivery to, a target site;
the one or more Nucleic Acid Agent, one or more Ligand, and one or more Nucleotide-Based Enhancement Unit are covalently linked to one another either directly or via a Linker Unit;
B is H, C₁-C₆ alkyl, or a nucleobase moiety;
V is —O—, —NR^{V}—, or —C(R^{V})₂—;
each R^{V} independently is H or C₁-C₆ alkyl;
X is H, halogen, or —OR^{X};
R^{X} is H, C₁-C₁₂ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Xa}; or R^{X} and R⁴ together form C₁-C₆ alkylene;
each R^{Xa} independently is halogen, C₁-C₆ alkyl, or —O—(C₁-C₆ alkyl), wherein the C₁-C₆ alkyl or —O—(C₁-C₆ alkyl) is optionally substituted with one or more halogen;
Y is —P(R^{Y})—, —P(OR^{Y})—, —P(N(R^{Y})₂)—, —P(═O)(OR^{Y})—, —P(═O)(R^{Y})—, —P(═S)(OR^{Y})—, —P(═S)(R^{Y})—, —P(═O)(SR^{Y})—, or —P(═S)(SR^{Y})—;
each R^{Y} independently is H or C₁-C₆ alkyl optionally substituted with one or more halogen or cyano;
R¹ is H, halogen, or C₁-C₆ alkyl optionally substituted with one or more halogen;
R² is H, halogen, or C₁-C₆ alkyl optionally substituted with one or more halogen;
R³ is H, halogen, or C₁-C₆ alkyl optionally substituted with one or more halogen;
R⁴ is H, halogen, or C₁-C₆ alkyl optionally substituted with one or more halogen; or R⁴ and R^{X} together form C₁-C₆ alkylene;
each R⁵ independently is H, halogen, or C₁-C₆ alkyl optionally substituted with one or more halogen;
each # independently denotes an attachment to the 2′- or 3′-position of another nucleotide of the Nucleotide-Based Enhancement Unit, or denotes H or an attachment to the rest of the conjugate when the nucleotide is at the 5′-end of the Nucleotide-Based Enhancement Unit; and
each ## independently denotes an attachment to the 5′-position of another nucleotide of the Nucleotide-Based Enhancement Unit, or denotes H or an attachment to the rest of the conjugate when the nucleotide is at the 2′- or 3′-end of the Nucleotide-Based Enhancement Unit.

2. The conjugate of any one of the preceding claims, wherein at least one Nucleotide-Based Enhancement Unit comprises from 3 to 20 nucleotides.

3. The conjugate of any one of the preceding claims, wherein at least one Nucleotide-Based Enhancement Unit comprises from 6 to 15 nucleotides.

4. The conjugate of any one of the preceding claims, wherein at least one Nucleotide-Based Enhancement Unit comprises 9 nucleotides.

5. The conjugate of any one of the preceding claims, wherein at least one Nucleotide-Based Enhancement Unit is attached to the Nucleic Acid Agent directly or via a Linker Unit.

6. The conjugate of any one of the preceding claims, wherein at least one Nucleotide-Based Enhancement Unit is attached to the sense strand of the Nucleic Acid Agent directly or via a Linker Unit.

7. The conjugate of any one of the preceding claims, wherein at least one Nucleotide-Based Enhancement Unit is attached to the 3′-end of the Nucleic Acid Agent directly or via a Linker Unit.

8. The conjugate of any one of the preceding claims, wherein at least one Nucleotide-Based Enhancement Unit does not bind to any part of the antisense strand of the Nucleic Acid Agent.

9. The conjugate of any one of the preceding claims, wherein at least one Nucleotide-Based Enhancement Unit is attached to a Ligand directly or via a Linker Unit.

10. The conjugate of any one of the preceding claims, wherein at least one Nucleotide-Based Enhancement Unit is attached to:
(i) both the Nucleic Acid Agent and the Ligand directly;
(ii) the Nucleic Acid Agent directly and the Ligand via a Linker Unit;
(iii) the Ligand directly and the Nucleic Acid Agent via a Linker Unit; or
(iv) both the Nucleic Acid Agent and the Ligand via a Linker Unit.

11. The conjugate of any one of the preceding claims, wherein the Nucleic Acid Agent is a double stranded RNA.

12. The conjugate of any one of the preceding claims, wherein the conjugate has a structure described in FIG. 1.

13. The conjugate of any one of the preceding claims, wherein the conjugate has a structure described in Table D.

14. The conjugate of any one of the preceding claims, wherein the conjugate has a structure described in Table C.

15. The conjugate of any one of the preceding claims, wherein the ligand comprises a carbohydrate moiety.

16. The conjugate of any one of the preceding claims, wherein the carbohydrate moiety comprises a monosaccharide, a disaccharide, a trisaccharide, or a tetrasaccharide.

17. The conjugate of any one of the preceding claims, wherein the carbohydrate moiety comprises galactose or a derivative thereof.

18. The conjugate of any one of the preceding claims, wherein the ligand comprises a moiety capable of binding to a glucagon-like peptide-1 receptor (GLP-1 receptor).

19. The conjugate of any one of the preceding claims, wherein the ligand comprises glucagon-like peptide-1 (GLP-1) or a derivative thereof.

20. The conjugate of any one of the preceding claims, wherein the ligand comprises glucagon or a derivative thereof.

21. An isotopic derivative of the conjugate of any one of the preceding claims.

22. A pharmaceutical composition comprising the conjugate of any one of the preceding claims.

23. A method of modulating the expression of a target gene in a subject, comprising administering to the subject the conjugate of any one of claims 1-21.

24. A method of delivering a Nucleic Acid Agent to a subject, comprising administering to the subject the conjugate of any one of claims 1-21.

25. A method of treating or preventing a disease in a subject in need thereof, comprising administering to the subject a therapeutically effective amount of the conjugate of any one of claims 1-21. [chemical structure]
